(19) United States
(12) Patent Application Publication
(10) Pub. No.: US 2007/0043035 A1
(43) Pub. Date: Feb. 22, 2007

(54) Novel compounds and their use in medicine: process for their preparation and pharmaceutical compositions containing them

(76) Inventors: Ranga Madhavan Gurram, Hyderabad (IN); Debnath Bhuniya, Hyderabad (IN); Saibal Kumar Das, Hyderabad (IN); Sudhir Kumar Sharma, Hyderabad (IN); Ranjan Chakrabarti, Hyderabad (IN); Javed Iqbal, Hyderabad (IN)

Correspondence Address: Milagros A. Cepeda;Dr. Reddy's Laboratories, Inc., 200 Somerset Corporate Blvd., Seventh Floor, Bridgewater, NJ, 08807-2862, US

(21) Appl. No.: 10/575,122

(22) PCT Filed: Jan. 29, 2004
(86) PCT No.: PCT/IB04/00208

§ 371 Date: Apr. 7, 2006

(30) Foreign Application Priority Data
Oct. 28, 2003 (IB) 03/04741

(51) Int. Cl. A61K 31/5415; A61K 31/5377; A61K 31/47; A61K 31/405; A61K 31/4035

(52) U.S. Cl. 514/224.2; 514/230.5; 514/311; 514/310; 514/414; 514/415; 514/419; 514/563; 544/51; 544/105; 546/146; 546/169; 548/465; 548/492

(57) ABSTRACT

The present invention relates to novel hypolipidemic, antiobesity, hypocholesterolemic and antidiabetic compounds. More particularly, the present invention relates to novel alkyl carboxylic acids of the general formula (I), their stereoisomers, pharmaceutically acceptable salts thereof and pharmaceutical compositions containing them where all symbols are as defined in the description.

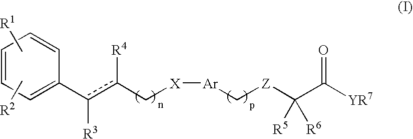

### FIELD OF THE INVENTION

The present invention relates to novel hypolipidemic, antiobesity, hypocholesterolemic and antidiabetic compounds. More particularly, the present invention relates to novel alkyl carboxylic acids of the general formula (I), their stereoisomers, pharmaceutically acceptable salts thereof and pharmaceutical compositions containing them. 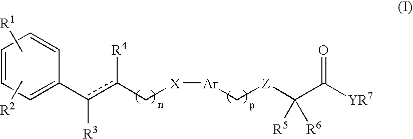
wherein R¹ and R² may be same or different and independently represent hydrogen, halogen, nitro, cyano, amino, hydroxy or optionally substituted group selected from alkyl, cycloalkyl, alkoxy, cycloalkoxy, aryl, aralkyl, alkylcarbonyl, alkoxycarbonyl, arylcarbonyl, aryloxycarbonyl, aralkoxycarbonyl, heteroarylcarbonyl, aryloxy, aralkoxy, alkylcarbonyloxy, alkoxycarbonylamino, aryloxycarbonylamino, aralkoxycarbonylamino, heteroarylcarbonylamino, heteroaryl, heteroaralkyl, heterocyclyl, heteroaralkoxy, heteroaryloxy, fluorenylmethoxycarbonyl (Fmoc), fluorenylmethoxycarbonylamino (N-Fmoc), —OSO₂R⁸, —OCONR⁸R⁹, NR⁸COOR⁹, —NR⁸COR⁹, —NR⁵R⁹, —NR⁸SO₂R⁹, —NR⁸CONR⁹R¹⁰, —NR⁸CSNR⁸R⁹, —SO₂R⁸, —SOR⁸, —SR⁸, —SO₂NR⁸R⁹, —SO₂OR⁸, —CONR⁸R⁹, —COOR⁹ or —COR⁹, wherein R⁸, R⁹ and R¹⁰ may be same or different and independently represent hydrogen, optionally substituted group selected from alkyl, aryl, aralkyl, aryloxy or heteroaryl; or R¹ and R² together represent a monocyclic or polycyclic aromatic or non aromatic ring or an aromatic ring fused to a non aromatic ring, which may optionally contain 1 to 3 heteroatoms selected from N, S, or O and may be unsubstituted or have 1 to 4 substituents which may be identical or different.
R³ and R⁴ may be same or different and independently represent hydrogen, halogen, optionally substituted group selected from alkyl, cycloalkyl, alkanoyl, aryl, aroyl, aralkyl or aralkanoyl group. ‘n’ and ‘p’ independently represents 0-6.
X represents O, S, NR where R represents hydrogen or optionally substituted groups selected from alkyl, cycloalkyl, cycloalkylalkyl, aryl, aralkyl, alkanoyl, or aroyl.
Ar represents optionally substituted single or fused aromatic, heteroaromatic or heterocyclic group.
Z represents O, S, NR where R is as defined above.
R⁵, R⁶ and R⁷ may be same or different and independently represent hydrogen, hydroxy, halogen or optionally substituted group selected from alkyl, cycloalkyl, alkoxy, aryl, aralkyl, heteroaryl, heterocyclyl or heteroaralkyl groups. R⁵ and R⁶ together may form a 5 or 6 membered cyclic rings, which may contain one or two hetero atoms selected from O, S or N.
Y represents O or NR¹¹ where R¹¹ represents hydrogen, optionally substituted group selected from alkyl, aryl, aralkyl, alkanoyl, aroyl, aralkanoyl, heterocyclyl or heteroaryl.
R⁷ and R¹¹ together may also form a 5 or 6 membered cyclic ring, which may contain one or two hetero atoms selected from O, S or N.
‘- - - ’ represents a bond or no bond.

The present invention also relates to a process for the preparation of the above said compounds.

The compounds of the present invention, increase high density lipoprotein (HDL) and decrease low density lipoprotein (LDL), triglycerides, lower total cholesterol (TC), and lower plasma glucose which have a beneficial effect on coronary heart disease and atherosclerosis.

The compounds of general formula (I) are useful in reducing body weight and for the treatment and/or prophylaxis of diseases such as atherosclerosis, stroke, peripheral vascular diseases and related disorders. These compounds are useful for the treatment of hyperlipidemia, hyperglycemia, hypercholesterolemia, lowering of atherogenic lipoproteins, VLDL (very low density lipoprotein) and LDL. The compounds of the present invention can be used for the treatment of renal diseases including glomerulonephritis, glomerulosclerosis, nephrotic syndrome, hypertensive nephrosclerosis and nephropathy. The compounds of general formula (I) are also useful for the treatment and/or prophylaxis of leptin resistance, impaired glucose tolerance, disorders related to syndrome X such as hypertension, obesity, insulin resistance, coronary heart disease and other cardiovascular disorders. These compounds may also be useful as aldose reductase inhibitors, for improving cognitive functions in dementia, treating diabetic complications, disorders related to endothelial cell activation, psoriasis, polycystic ovarian syndrome (PCOS), inflammatory bowel diseases, osteoporosis, myotonic dystrophy, pancreatitis, arteriosclerosis, retinopathy, xanthoma, eating disorders, inflammation and for the treatment of cancer. The compounds of the present invention are also useful in the treatment and/or prophylaxis of the above said diseases in combination/concomittant with one or more HMG CoA reductase inhibitor, cholesterol absorption inhibitor; antiobesity drug; lipoprotein disorder treatment drug; hypoglycemic agent: insulin; biguanide; sulfonylurea; thiazolidinedione; dual PPARα and γ or a mixture thereof.

### BACKGROUND OF THE INVENTION

Atherosclerosis and other peripheral vascular diseases affect the quality of life of millions of people. Therefore, considerable attention has been directed towards understanding the etiology of hypercholesterolemia and hyperlipidemia and development of effective therapeutic strategies.

Statins and fibrates are the more widely used drugs for the treatment of the hyperlipidemia. Statins act via HMG CoA reductase enzyme there by cholesterol biosynthesis. The predominant effect of statins is lowering the levels of LDL cholesterols (LDL-C). Fibrates another class of hyperlipidemic compounds are known to be weak agonist of Peroxisome Proliferator Activated Receptor (PPAR)-α subtypes. Peroxisome proliferator activated receptors (PPARs) are members of the nuclear receptor super family. The gamma (γ) isoform of PPAR (PPARγ) has been implicated in regulating differentiation of adipocytes (Endocrinology, 135 (1994) 798-800) and energy homeostasis (Cell, 83 (1995) 803-812), whereas the alpha (α) isoform of PPAR (PPARα) mediates fatty acid oxidation (Trend. Endocrin. Metab., 4 (1993) 291-296) thereby resulting in reduction of circulating free fatty acid in plasma (Current Biol. 5 (1995) 618-621). PPARα agonists have been found useful for the treatment of obesity (WO 97/36579). A wealth of information exists on the influence of fibrates as PPAR-α agonists on the cardiovascular risk profile. These compounds correct atherogenic dyslipoproteinemia. Several angiographic intervention trials show a decreases incidence of cardiovascular events (Trends in Pharmaceutical Sciences 2001, 22(9), 441-443). It has been recently disclosed that compounds, which are agonists for both PPARα and PPARγ are suggested to be useful for the treatment of syndrome X (WO 97/25042). Similar effect between the insulin sensitizer (PPARγ agonist) and HMG CoA reductase inhibitor has been observed which may be useful for the treatment of atherosclerosis and xanthoma (EP 0 753 298).

It is known that PPARγ plays an important role in adipocyte differentiation (Cell, 87 (1996) 377-389). Ligand activation of PPAR is sufficient to cause complete terminal differentiation (Cell, 79 (1994) 1147-1156) including cell cycle withdrawal. PPARγ is consistently expressed in certain cells and activation of this nuclear receptor with PPARγ agonists would stimulate the terminal differentiation of adipocyte precursors and cause morphological and molecular changes characteristics of a more differentiated, less malignant state (Molecular Cell, (1998), 465470; Carcinogenesis, (1998), 1949-53; Proc. Natl. Acad. Sci., 94 (1997) 237-241) and inhibition of expression of prostate cancer tissue (Cancer Research 58 (1998) 3344-3352). This would be useful in the treatment of certain types of cancer, which express PPARγ and could lead to a quite nontoxic chemotherapy.

Hypercholesterolemia has been defined as plasma cholesterol level that exceeds arbitrarily defined value called “normal” level. Recently, it has been accepted that “ideal” plasma levels of cholesterol are much below the “normal” level of cholesterol in the general population and the risk of coronary artery disease (CAD) increases as cholesterol level rises above the “optimum” (or “ideal”) value. There is clearly a definite cause and effect-relationship between hypercholesterolemia and CAD, particularly for individuals with multiple risk factors. Most of the cholesterol is present in the esterified forms with various lipoproteins such as Low density lipoprotein (LDL), Intermediate density lipoprotein (IDL), High density lipoprotein (HDL) and partially as Very low density lipoprotein (VLDL). Studies clearly indicate that there is an inverse correlationship between CAD and atherosclerosis with serum HDL-cholesterol concentrations (Stampfer et al., N. Engl. J. Med., 325 (1991), 373-381). The risk of CAD increases with increasing levels of LDL and VLDL.

Atherosclerosis coronary artery disease is fast becoming a major cause for mortality both the developing and developed nations. It has been demonstrated that abnormal cholesterol levels play a major role for morbidity and mortality, and aggressive treatment saves lives. Clinical trials have demonstrated convincing benefits of cholesterol lowering, for reducing myo cardial infarction among patients with CHD as well as for decreasing the incidents of cardiac events in patients without established coronary disease (JAMA 2001, 285 (19), 2508-2509).

In CAD, generally “fatty streaks” in carotid, coronary and cerebral arteries, are found which are primarily free and esterified cholesterol. Miller et al., (Br. Med. J., 282 (1981), 1741-1744) have shown that increase in HDL-particles may decrease the number of sites of stenosis in coronary arteries of human, and high level of HDL-cholesterol may protect against the progression of atherosclerosis. Picardo et al., Arteriosclerosis 6 (1986) 434-441 have shown by in vitro experiment that HDL is capable of removing cholesterol from cells. They suggest that HDL may deplete tissues of excess free cholesterol and transfer it to liver, which is known as reverse cholesterol transport, (Macikinnon et al., J. Biol. chem. 261 (1986), 2548-2552). Therefore, agents that increase HDL cholesterol would have therapeutic significance for the treatment of hypercholesterolemia and coronary heart diseases (CHD).

Obesity is a disease highly prevalent in affluent societies and in the developing world and is a major cause of morbidity and mortality. It is a state of excess body fat accumulation. The causes of obesity are unclear. It is believed to be of genetic origin or promoted by an interaction between the genotype and environment. Irrespective of the cause, the result is fat deposition due to imbalance between the energy intake versus energy expenditure. Dieting, exercise and appetite suppression have been a part of obesity treatment. There is a need for efficient therapy to fight this disease since it may lead to coronary heart disease, diabetes, stroke, hyperlipidemia, gout, osteoarthritis, reduced fertility and many other psychological and social problems.

Diabetes and/or insulin resistance is yet another disease which severely effects the quality of large population in the world. Insulin resistance is the diminished ability of insulin to exert its biological action across a broad range of concentrations. In insulin resistance, the body secretes abnormally high amounts of insulin to compensate for this defect; failing which, the plasma glucose concentration inevitably raises and develops into diabetes. Among the developed countries, diabetes mellitus is a common problem and is associated with a variety of abnormalities including obesity, hypertension, hyperlipidemia (J. Clin. Invest., 75 (1985) 809-817; N. Engl. J. Med 317 (1987) 350-357; J. Clin. Endocrinol. Metab., 66 (1988) 580-583; J. Clin. Invest., 68 (1975) 957-969) and other renal complications (patent publication No. WO 95/21608). It is now increasingly being recognized that insulin resistance and relative hyperinsulinemia have a contributory role in obesity, hypertension, atherosclerosis and type 2 diabetes mellitus. The association of insulin resistance with obesity, hypertension and angina has been described as a syndrome having insulin resistance as the central pathogenic link-Syndrome-X.

Hyperlipidemia is the primary cause for cardiovascular (CVD) and other peripheral vascular diseases. High risk of CVD is related to the higher LDL (Low Density Lipoprotein) and VLDL (Very Low Density Lipoprotein) seen in hyperlipidemia. Patients having glucose intolerance/insulin resistance in addition to hyperlipidemia have higher risk of CVD. Numerous studies in the past have shown that lowering of plasma triglycerides and total cholesterol, in particular LDL and VLDL and increasing HDL cholesterol help in preventing cardiovascular diseases.

Leptin resistance is a condition wherein the target cells are unable to respond to leptin signal. This may give rise to obesity due to excess food intake and reduced energy expenditure and cause impaired glucose tolerance, type 2 diabetes, cardiovascular diseases and such other interrelated complications. Kallen et al (Proc. Natl. Acad. Sci. (1996) 93, 5793-5796) have reported that insulin sensitizers which perhaps due to the PPAR agonist expression lower plasma leptin concentrations. However, it has been recently disclosed that compounds having insulin sensitizing property also possess leptin sensitization activity. They lower the circulating plasma leptin concentrations by improving the target cell response to leptin (WO 98/02159).

Fibrates are a class of drugs which may lower serum triglycerides, lower LDL-C, shift the LDL particle size from the more atherogenic small dense to normal dense LDL-C and increase the HDL-C. Experimental evidence indicate that the effects of fibrates on serum lipids are mediated through activation of PPAR-α (Curr. Pharm. Des., 1-14, 3(1), 1997). Activation of PPAR-A results in transcription of enzymes that increases fatty acids catabolism and decrease denovo fatty acid synthesis in the liver resulting in decreased triglyceride synthesis in the liver resulting in decreased triglyceride synthesis and VLDL-C production. PPAR-α ligands may be useful for the treatment of dyslipidemia and cardiovascular disorders (Curr. Opin. Lipido., 1999, 10, 245-257).

Some of relevant compounds described in the prior art are outlined below:
(i) International publication no. WO 01/55085 A1 disclose the compound of general formula (IV) 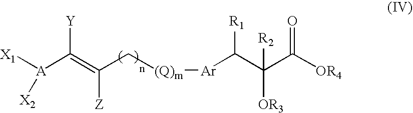
where all symbols are as defined in the PCT publication.
An example of the above compounds as shown in formula (IVb) 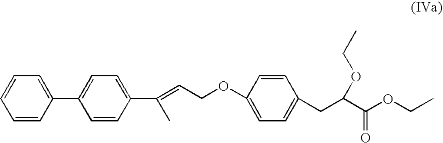
(ii) International publication no. WO 01/16120 A1 disclose the compound of general formula (V) 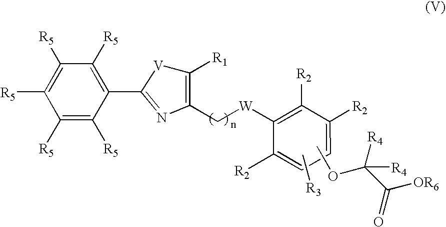
where all symbols are as defined in the PCT publication.
An example of the above compounds as shown in formula (Va) 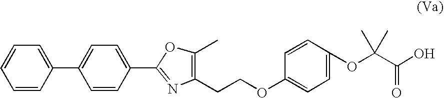
(iii) International publication No. WO 00/49005 disclose the compounds of general formula (VI) 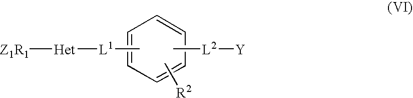
where all symbols are as defined in the PCT publication.
An example of these compounds is shown in formula (VIa) 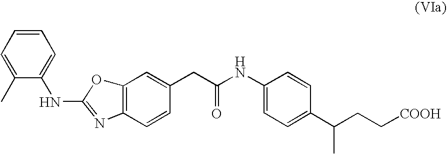
(iv) International publication No. WO 00/05223 disclose the compounds of general formula (X) 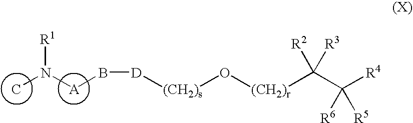
where all symbols are as defined earlier.
An example of these compounds is shown in formula (Xa) 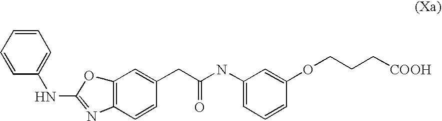
(v) International publication No. WO 00/64888 disclose the compounds of general formula (XI) 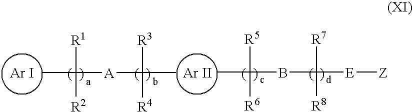
where all symbols are as defined earlier.
An example of these compounds is shown in formula (XIa)

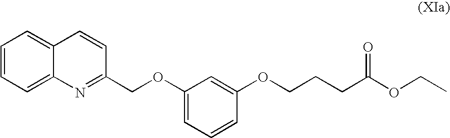

A number of compounds have been reported to be useful in the treatment of hyperglycemia, hyperlipidemia and hypercholesterolemia (PCT Publication nos. WO 99/16758, WO 99/19313, WO 99/08501, WO97/36579, WO 97/25042, WO 95/17394, WO 96/04260, WO 95/03038, WO 94/13650, WO 94/01420 etc.

### SUMMARY OF THE INVENTION

One aspect of the present invention is to provide a novel compound of the general formula (I), as defined above, having PPAR agonist activity.

Another aspect of the present invention is to provide a compound of formula (I), their stereoisomers, their pharmaceutically acceptable salts and pharmaceutical compositions

Yet another aspect of the present invention is to provide a process for the preparation of compounds of formula (I), as defined above.

Yet another aspect of the present invention relates to a process of separating (R)-isomer and (S) isomer from a mixture of (R) and (S) isomers of compound of formula (I).

Yet another aspect of the present invention is to provide a pharmaceutical composition, containing the compounds of the general formula (I) as defined above and one or more HMG CoA reductase inhibitors; cholesterol absorption inhibitors; antiobesity drugs; lipoprotein disorder treatment drugs; hypoglycemic agents: insulin; biguanides; sulfonylureas; thiazolidinediones; dual PPARα and γ or a mixture thereof in combination with the usual pharmaceutically employed carriers, diluents and the like.

Accordingly, the present invention provides novel compounds of formula (I), 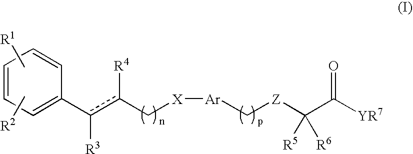
their stereoisomers, pharmaceutically acceptable salts, their pharmaceutical compositions thereof, wherein
R¹ and R² may be same or different and independently represent hydrogen, halogen, nitro, cyano, amino, hydroxy or optionally substituted group selected from alkyl, cycloalkyl, alkoxy, cycloalkoxy, aryl, aralkyl, alkylcarbonyl, alkoxycarbonyl, arylcarbonyl, aryloxycarbonyl, aralkoxycarbonyl, heteroarylcarbonyl, aryloxy, aralkoxy, alkylcarbonyloxy, alkoxycarbonylamino, aryloxycarbonylamino, aralkoxycarbonylamino, heteroarylcarbonylamino, heteroaryl, heteroaralkyl, heterocyclyl, heteroaralkoxy, heteroaryloxy, fluorenylmethoxycarbonyl (Fmoc), fluorenylmethoxycarbonylamino (N-Fmoc), —OSO₂R⁸, —OCONR⁸R⁹; NR⁸COOR⁹, —NR⁸COR⁹, —NR⁸R⁹, —NR⁸SO₂R⁹, —NR⁸CONR⁹R¹⁰, —NR⁸CSNR⁸R⁹, —SO₂R⁸, —SOR⁸, —SR⁸, —SO₂NR⁸R⁹, —SO₂OR⁸, —CONR⁸R⁹, —COOR⁹ or —COR⁹, wherein R⁸, R⁹ and R¹⁰ may be same or different and independently represent hydrogen, optionally substituted group selected from alkyl, aryl, aralkyl, aryloxy or heteroaryl; or R¹ and R² together represent a monocyclic or polycyclic aromatic or non aromatic ring or an aromatic ring fused to a non aromatic ring, which may optionally contain 1 to 3 heteroatoms selected from N, S, or O and may be unsubstituted or have 1 to 4 substituents which may be identical or different.
R³ and R⁴ may be same or different and independently represent hydrogen, halogen, optionally substituted group selected from alkyl, cycloalkyl, alkanoyl, aryl, aroyl, aralkyl or aralkanoyl group. ‘n’ and ‘p’ independently represents 0-6.
X represents O, S, NR where R represents hydrogen or optionally substituted groups selected from alkyl, cycloalkyl, cycloalkylalkyl, aryl, aralkyl, alkanoyl, or aroyl.
Ar represents optionally substituted single or fused aromatic, heteroaromatic or heterocyclic group.
Z represents O, S, NR where R is as defined above.
R⁵, R⁶ and R⁷ may be same or different and independently represent hydrogen, hydroxy, halogen or optionally substituted group selected from alkyl, cycloalkyl, alkoxy, aryl, aralkyl, heteroaryl, heterocyclyl or heteroaralkyl groups. R⁵ and R⁶ together may form a 5 or 6 membered cyclic rings, which may contain one or two hetero atoms selected from O, S or N.
Y represents O or NR¹¹ where R¹¹ represents hydrogen, optionally substituted group selected from alkyl, aryl, aralkyl, alkanoyl, aroyl, aralkanoyl, heterocyclyl or heteroaryl.
R⁷ and R¹¹ together may also form a 5 or 6 membered cyclic ring, which may contain one or two hetero atoms selected from O, S or N.

‘----’ represents a bond or no bond.

The substituents on the fused rings formed by R¹ and R² may be selected from alkyl, halogen, hydroxy, haloalkyl, nitro, amino, cyano, oxo, or thioxo.

The substituents on R¹ and R² are selected from halogen, hydroxy, nitro, amino, oxo, thioxo, optionally substituted groups selected from alkyl, cycloalkyl, alkoxy, aryl, aralkyl, alkylsulfonyl, alkylsulinyl, alkylsulfanyl, alkylsulfonyloxy, alkylsulfinyloxy or alkylsulfanyloxy, the substituents are selected from halogen, hydroxyl, nitro, amino, cyano or alkyl.

The substituents on R, R³, R⁴ and R¹¹ may be selected from halogen, nitro, amino, hydroxy, alkyl, oxo or aralkyl

The substituents on cyclic rings fromed by R⁵ and R⁶ are substituted, the substituents are selected from alkyl, halogen, hydroxy, haloalkyl, nitro, amino, cyano, oxo, or thioxo.

The substitutents on R⁵, R⁶ and R⁷ may be selected from halogen, hydroxy, nitro, alkyl, cycloalkyl, alkoxy, aryl, aralkyl, aralkoxyalkyl, heterocyclyl, heteroaryl or amino.

The groups defined for R, R¹, R², R³, R⁴, R⁵, R⁶, R⁷, R⁸, R⁹, R¹⁰ and R¹¹ may be unsubstiuted, or have 1 to 4 substituents, which may be identical or different.

### DETAILED DESCRIPTION OF THE INVENTION AND EMBODIMENTS

The groups defined for R, R¹, R², R³, R⁴, R⁵, R⁶, R⁷, R⁸, R⁹, R¹⁰, R¹¹ and the groups defined for substituents are defined as below:

‘Halogen’ group represents chlorine, fluorine, bromine or iodine.

‘Alkyl’ group is linear or branched (C₁-C₁₀)allyl group. Exemplary alkyl groups include methyl, ethyl, n-propyl, iso-propyl, n-butyl, iso-butyl, t-butyl, n-pentyl, iso-pentyl, hexyl, heptyl, octyl and the like, which may optionally be susbstituted.

‘Haloalkyl’ group is halogen-(C₁-C₁₀)alkyl group, where halogen and (C₁-C₁₀)alkyl groups are as defined above. Exemplary groups include chloromethyl, dichloromethyl, trifloromethyl and the like.

‘Cycloalkyl’ group is (C₃-C₁₀)cycloalkyl group. Exemplary cycloalkyl groups include cyclopropyl, cyclobutyl, cyclopentyl, cyclohexyl and the like, which may optionally be susbstituted.

‘Cycloalkylalkyl’ group is (C₃-C₁₀)cycloalkyl(C₁-C₁₀)alkyl group, where cycloalkyl and alkyl groups are as defined earlier. Exemplary cycloalkylalkyl groups include cyclopropyl-methyl, cyclobutyl-methyl, cyclopentyl-methyl, cyclohexyl-methyl and the like, which may optionally be susbstituted.

‘Alkoxy’ is (C₁-C₁₀)alkyl-O—, wherein (C₁-C₁₀)alkyl group is as defined above. Exemplary alkyl groups include methoxy, ethoxy, propyloxy, butyloxy, iso-propyloxy and the like, which may optionally be susbstituted.

‘Cycloalkoxy’ is (C₃-C₁₀)cycloalkoxy group. Exemplary cycloalkoxy groups include cyclopropoxy, cyclobutoxy, cyclopentoxy, cyclohexoxy and the like, which may optionally be susbstituted.

‘Alkanoyl’ is H—CO— or (C₁-C₁₀)alkyl-CO—, where (C₁-C₁₀)alkyl group is as defined above. Exemplary acyl groups include acetyl, propanoyl, butanoyl, pentanoyl, benzoyl and the like, which may optionally be susbstituted.

‘Aralkanoyl’ is aryl-alkanoyl group, where aryl and alkanoyl groups are as defined earlier. The exemplary aralkanoyl groups include phenylpropanoyl, phenylbutanoyl, phenylpentanoyl and the like, which may optionally be susbstituted.

‘Aryl’ is monocylic or multicyclic ring system having about 6 to 14 carbon atoms. Exemplary groups include phenyl, naphthyl and like, which may optionally be susbstituted.

‘Aryloxy’ is aryl-O— group, where aryl group is as defined above. Exemplary aryloxy groups include phenoxy, naphthyloxy and the like, which may optionally be susbstituted.

‘Aroyl’ is aryl-CO— group. Exemplary aroyl groups include benzoyl, 1-naphthoyl and the like, which may optionallt substituted.

‘Aralkyl’ is benzyl, 2-phenethyl and the like, which may optionally be susbstituted.

‘Aralkoxy’ is aralkyl-O— group, wherein the aralkyl group as defined above. Exemplary aralkoxy groups include benzyloxy, 2-phenethyloxy and the like, which may optionally be susbstituted.

‘Heterocyclyl’ is a non-aromatic saturated monocyclic or multicyclic ring system having about 5 to about 10 carbon atoms, having at least one hetero atom selected from O, S or N. Exemplary heterocyclyl groups include aziridinyl, pyrrolidinyl, piperidinyl, piperazinyl, morpholinyl, thiomorpholinyl, thiazolidinyl, 1,3-dioxolanyl, 1,4-dioxanyl and the like, which may optionally be susbstituted.

‘Heteroaralkoxy’ is heteroaralkyl-O—, wherein heteroaralkyl group is as defined above. Exemplary heteroaralkoxy groups include thienylmethyloxy, pyridylmethyloxy and the like, which may optionally be susbstituted.

‘Heteroaryloxy’ is heteroaryl-O—, wherein heteroaryl group is as defined above. Exemplary heteroaryloxy groups include pyrazinyloxy, isothiazolyloxy, oxazolyloxy, pyrazolyloxy, pyridazinyloxy, phthalazinyloxy, indolyloxy, quinazolinyloxy, pyridyloxy, thienyloxy and the like, which may optionally be susbstituted.

‘Heteroaryl’ is an aromatic monocyclic or multicyclic ring system having about 5 to about 10 carbon atoms, having at least one heteroatom selected from O, S or N. Exemplary heteroaryl groups include as pyrazinyl, isothiazolyl, oxazolyl, pyrazolyl, pyrrolyl, pyridazinyl, thienopyrimidyl, furyl, indolyl, isoindolyl, 1,3-benzodioxole, 1,3-benzoxathiole, quinazolinyl, pyridyl, thiophenyl and the like, which may optionally be susbstituted.

‘Heteroaralkyl’ is heteroaryl-(C₁-C₁₀)alkyl group, wherein the heteroaryl and (C₁-C₁₀)alkyl groups are as defined above. Exemplary heteroaralkyl groups include thienylmethyl, pyridylmethyl, imidazolylmethyl and the like, which may optionally be susbstituted.

Alkylcarbonyl’ is (C₁-C₁₀)alkyl-CO—, wherein (C₁-C₁₀)alkyl group is as defined above. Exemplary alkylcarbonyl groups include methylcarbonyl, ethylcarbonyl, propylcarbonyl and the like, which may optionally be susbstituted.

‘Alkylcarbonyloxy’ is (C₁-C₁₀)alkyl-CO—O, wherein (C₁-C₁₀)alkyl group is as defined above. Exemplary alkylcarbonyloxy groups include methylcarbonyloxy, ethylcarbonyloxy, propylcarbonyloxy and the like, which may optionally be susbstituted.

‘Alkoxycarbonyl’ is (C₁-C₁₀)alkyl-O—CO—, wherein (C₁-C₁₀)alkyl group is as defined above. Exemplary alkoxycarbonyl groups include methoxycarbonyl, ethoxycarbonyl, t-butoxycarbonyl and the like, which may optionally be susbstituted.

‘Alkoxycarbonylamino’ is (C₁-C₁₀)alkyl-O—CO-amino, wherein (C₁-C₁₀)alkyl group is as defined above. Exemplary alkoxycarbonyl groups include methoxycarbonylamino, ethoxycarbonylamino, t-butoxycarbonylamino and the like, which may optionally be susbstituted.

‘Arylcarbonyl’ is aryl-CO—, wherein aryl group is as defined above. Exemplary arylcarbonyl groups include phenylcarbonyl, naphthylcarbonyl and the like, which may optionally be susbstituted.

‘Aryloxycarbonyl’ is aryl-O—CO—, wherein aryl group is as defined above. Exemplary aryloxycarbonyl groups include phenoxycarbonyl, naphthyloxycarbonyl and the like, which may optionally be susbstituted.

‘Alkylsulfonyl’ is (C₁-C₁₀)alkylsulfonyl, where (C₁-C₁₀)alkyl group is as defined above. Exemplary alkylsulfonyl groups include methylsulfonyl, ethylsulfonyl and the like

‘Alkylsulfinyl’ is (C₁-C₁₀)alkylsulfinyl, where (C₁-C₁₀)alkyl group is as defined above. Exemplary alkylsulfinyl groups include methylsulfinyl, ethylsulfinyl and the like

‘Alkylsulfanyl’ is (C₁-C₁₀)alkylsulfanyl, where (C₁-C₁₀)alkyl group is as defined above. Exemplary alkylsulfanyl groups include methylsulfanyl, ethylsulfanyl and the like

‘Alkylsulfonyloxy’ is (C₁-C₁₀)alkylsulfonyloxy, where (C₁-C₁₀)alkyl group is as defined above. Exemplary alkylsulfonyloxy groups include methylsulfonyloxy, ethylsulfonyloxy and the like.

‘Alkylsulfanyloxy’ is (C₁-C₁₀)alkylsulfanyloxy, where (C₁-C₁₀)alkyl group is as defined above. Exemplary alkylsulfanyloxy groups include methylsulfanyloxy, ethylsulfanyloxy and the like.

Alkylsulfinyloxy’ is (C₁-C₁₀)alkylsulfinyloxy, where (C₁-C₁₀)alkyl group is as defined above. Exemplary alkylsulfinyloxy groups include methylsulfinyloxy, ethylsulfinyloxy and the like.

‘Aryloxycarbonylamino’ is aryl-O—CO-amino, wherein aryl group is as defined above. Exemplary aryloxycarbonyl groups include phenoxycarbonylamino, naphthyloxycarbonylamino and the like, which may optionally be susbstituted.

‘Aralkoxycarbonyl’ is aralkoxy-CO—, where aralkoxy is as defined above. Exemplary aralkoxycarbonyl groups include benzyloxycarbonyl, 2-phenethyloxycarbonyl and the like, which may optionally be susbstituted.

‘Aralkoxyalkyl’ is aralkoxy-(C₁-C₁₀)alkyl, where aralkoxy and (C₁-C₁₀)alkyl are as defined above. Exemplary aralkoxyalkyl groups include benzyloxymethyl, benzyloxyethyl, 2-phenethyloxyethyl and the like, which may optionally be susbstituted.

‘Aralkoxycarbonylamino’ is aralkoxy-CO-amino, where aralkoxy are as defined above. Exemplary aralkoxycarbonyl groups include benzyloxycarbonylamino, 2-phenethyloxycarbonylamino and the like, which may optionally be susbstituted.

‘Heteroarylcarbonyl’ is heteroaryl-CO—, wherein heteroaryl is as defined above. Exemplary heteroarylcarbonyl groups include pyrazinylcarbonyl, isothiazolylcarbonyl, oxazolylcarbonyl, pyrazolylcarbonyl, pyrrolylcarbonyl, pyridazinylcarbonyl, indolylcarbonyl and the like, which may optionally be susbstituted.

‘Heteroarylcarbonylamino’ is heteroaryl-CO-amino, wherein heteroaryl is as defined above. Exemplary heteroarylcarbonylamino groups include pyrazinylcarbonylamino, isothiazolylcarbonylamino, oxazolylcarbonylamino, pyrazolylcarbonylamino, pyrrolylcarbonylamino, pyridazinylcarbonylamino, indolylcarbonylamino and the like, which may optionally be susbstituted.

‘Ar’ may be selected from optionally substituted groups selected from phenylene, naphthylene, pyridyl, quinolinyl, benzofuryl, dihydrobenzofuryl, benzopyranyl, dihydrobenzopyranyl, indolyl, indolinyl, azaindolyl, azaindolinyl, pyrazolyl, benzothiazolyl, benzoxazolyl and the like. The substituents on the group represented by Ar may be selected from linear or branched optionally halogenated (C₁-C₁₀)alkyl, optionally halogenated (C₁-C₁₀)alkoxy, halogen, acyl, amino, acylamino, thio or carboxylic or sulfonic acids and their derivatives, which may optionally be susbstituted.

It is more preferred that ‘Ar’ represent optionally substituted phenylene, naphthylene, benzofuryl, indolyl, indolinyl, quinolinyl, azaindolyl, azaindolinyl, benzothiazolyl or benzoxazolyl groups.

It is still more preferred that ‘Ar’ is represented by phenylene, naphthylene or benzofuryl, which may be unsubstituted or substituted by alkyl, haloalkyl, methoxy or haloalkoxy groups.

Cyclic rings formed by R⁵ and R⁶ together may form a optionally susbstituted 5 or 6 membered cyclic rings selected from cyclopropyl, cyclobutyl, cyclopentyl, cyclohexyl and the like; pyrrolidinyl, piperidinyl, morpholinyl, piperazinyl and the like.

R¹ and R² together represent a optionally substituted monocyclic or polycyclic aromatic or non aromatic ring or an aromatic ring fused to a non aromatic ring selected from, 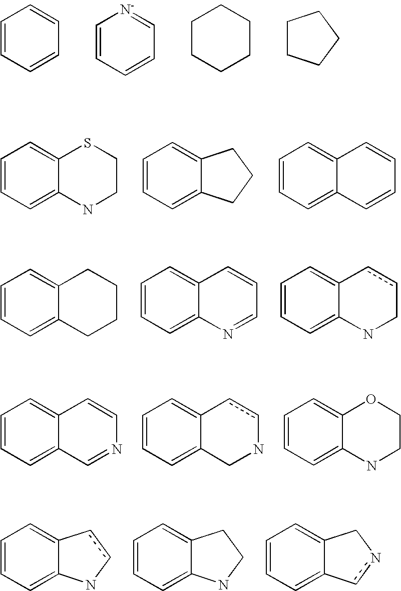
and the like.

Unless defined otherwise, all technical and scientific terms used herein have the same meaning as commonly understood to one of ordinary skill in the art to which this invention belongs. Although any methods, devices, and materials similar or equivalent to those described herein can be used in the practice or testing of the invention, the preferred methods, devices and materials are now described.

According to an embodiment of the present invention, there is provided a compound of formula (I), their pharmaceutically acceptable salts, their stereoisomers and their pharmaceutical compositions, wherein:

R¹ and R² may be same or different and independently represent hydrogen, halogen, nitro, cyano, amino, hydroxy or optionally substituted group selected from alkyl, alkoxy, aryl, aralkyl, aralkoxy, heteroaryl, heteroaralkoxy, —OSO₂R⁸, —SO₂R⁸ or —NR⁸R⁹;

R³ and R⁴ may be same or different and independently represent hydrogen, halogen, optionally substituted group selected from alkyl or aralkyl;

R⁵, R⁶ and R⁷ may be same or different and independently represent hydrogen, hydroxy, optionally substituted selected from alkyl, cycloalkyl, aryl or R⁵ and R⁶ together represent a 5 or 6 membered aromatic or non aromatic cyclic ring system optionally containing 1 or 2 heteroatoms selected from O, S or N;

R⁷ and R¹¹ may form a cyclic ring system selected from pyrrolidinyl, piperidinyl, morpholinyl, piperazinyl, oxazolinyl, diazolinyl and the like.

According to another embodiment of the present invention, there is provided a compound of formula (I), their pharmaceutically acceptable salts, their stereoisomers and their pharmaceutical compositions, wherein:
R¹ and R² together represent a optionally susbstituted monocyclic or polycyclic aromatic or non aromatic ring or an aromatic ring fused to a non aromatic ring selected from:

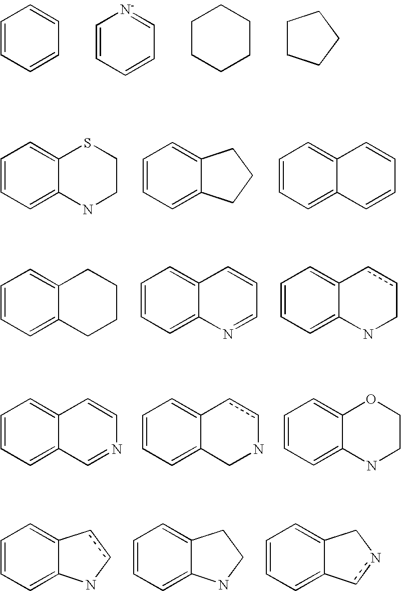

According to yet another embodiment of the present invention, there is provided a compound of formula (I), their pharmaceutically acceptable salts, their stereoisomers and their pharmaceutical compositions, wherein:

R¹ and R² may be same or different and independently represent hydrogen, halogen, nitro, amino, hydroxy or optionally substituted group selected from alkyl, aryl, aralkyl, aralkoxy, heteroaryl, heteroaralkoxy or —OSO₂R⁸;

R³ and R⁴ may be same or different and independently represent hydrogen or optionally substituted alkyl;

R⁵, R⁶ and R⁷ may be same or different and independently represent hydrogen, optionally substituted group selected from alkyl, cycloalkyl, aryl or R⁵ and R⁶ together represent a 5 or 6 membered saturated cyclic ring system;

According to still another embodiment of the present invention, there is provided a compound of formula (I), their pharmaceutically acceptable salts, their stereoisomers and their pharmaceutical compositions, wherein:
R¹ and R² together represent a optionally substituted monocyclic or polycyclic aromatic or non aromatic ring or an aromatic ring fused to a non aromatic ring selected from: 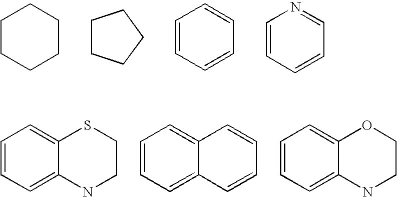
R³ and R⁴ may be same or different and independently represent hydrogen, optionally substituted alkyl;
R⁵, R⁶ and R⁷ may be same or different and independently represent hydrogen, optionally substituted group selected from alkyl, cycloalkyl, aryl or R⁵ and R⁶ together represent a 5 or 6 membered saturated cyclic ring system;

According to yet another embodiment of the present invention, there is provided a compound of formula (I), their pharmaceutically acceptable salts, their stereoisomers and their pharmaceutical compositions, wherein:

R¹ is selected from —SO₂CH₃, halogen, alkyl optionally substituted phenyl wherein the substituent is selected from alkyl or halogen

R², R³, R⁴, R⁵, R⁶ and R⁷ may be same or different and independently represent hydrogen, methyl, ethyl or propyl

‘Ar’ represents optionally substituted phenyl wherein the substituent is alkyl x Y and Z independently represent oxygen

n and p independently represent 0 or 1

According to still another embodiment of the present invention, there is provided a compound of formula (I), their pharmaceutically acceptable salts, their stereoisomers and their pharmaceutical compositions, wherein:

R¹ is selected from optionally substituted phenyl wherein the substituent is selected from halogen

R², R³, R⁴, R⁵, R⁶ and R⁷ may be same or different and independently represent hydrogen, methyl, ethyl or propyl

‘Ar’ represents optionally substituted phenyl wherein the substituent is alkyl

X, Y and Z independently represent oxygen

n and p independently represent 0 or 1.

One group of preferred compounds of the formula (I) are: 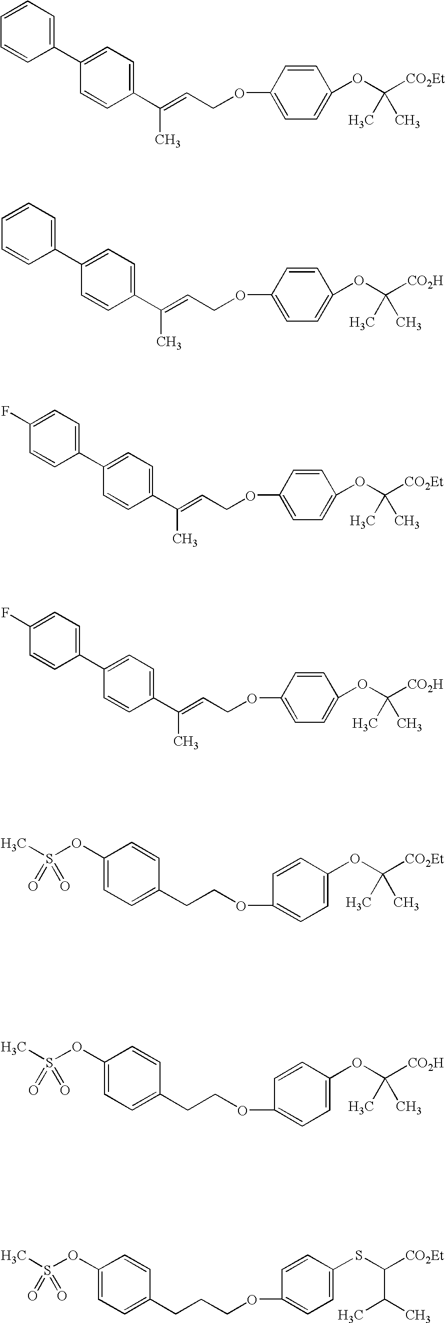 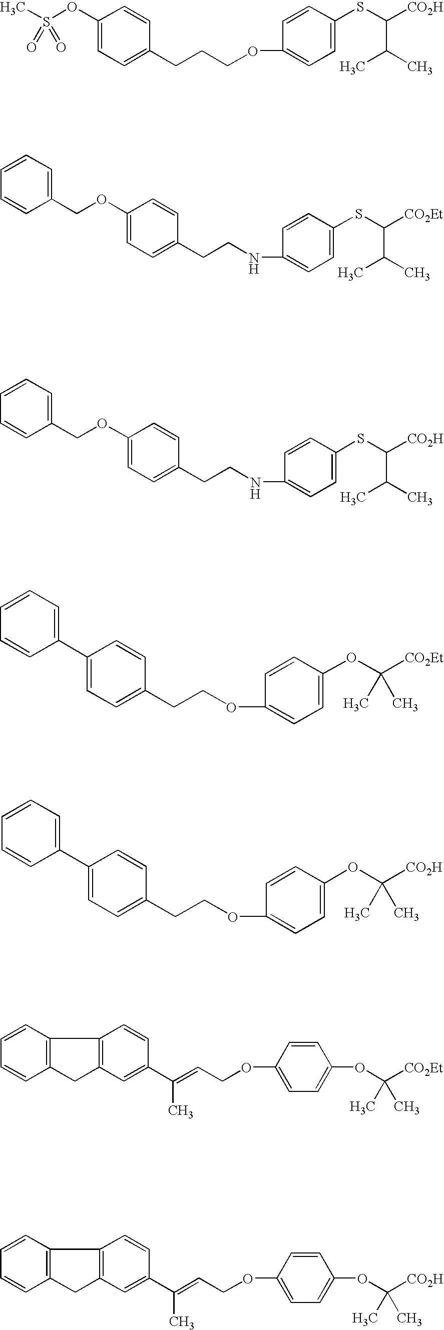 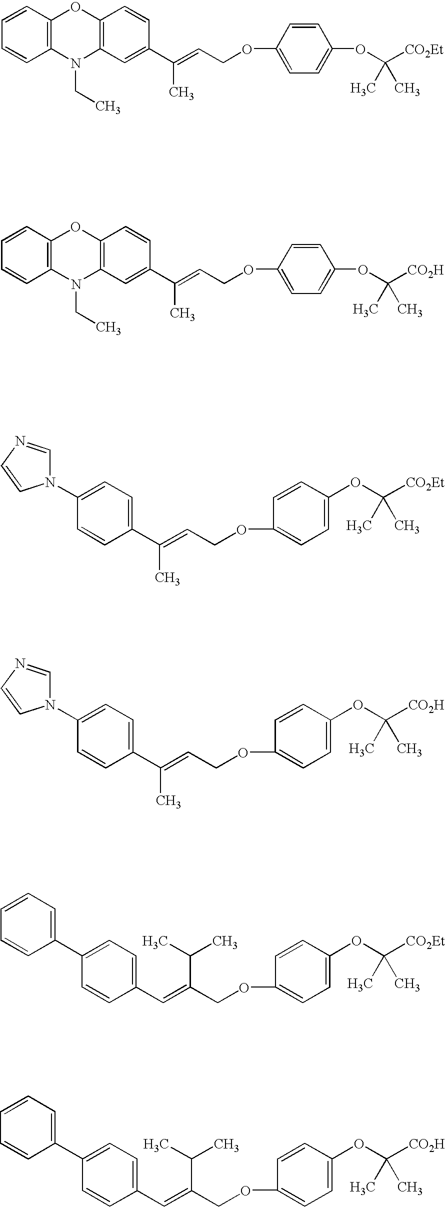 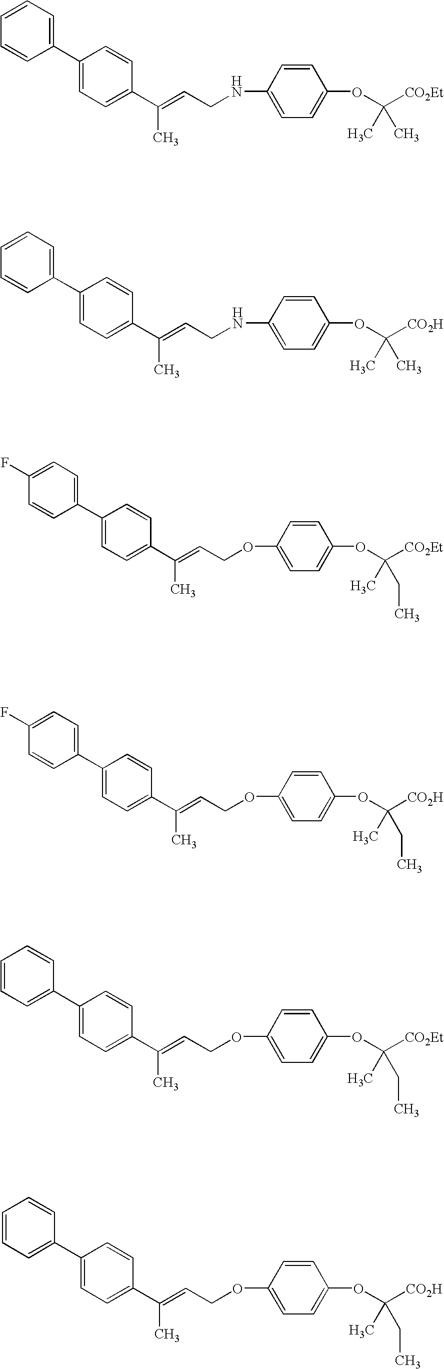 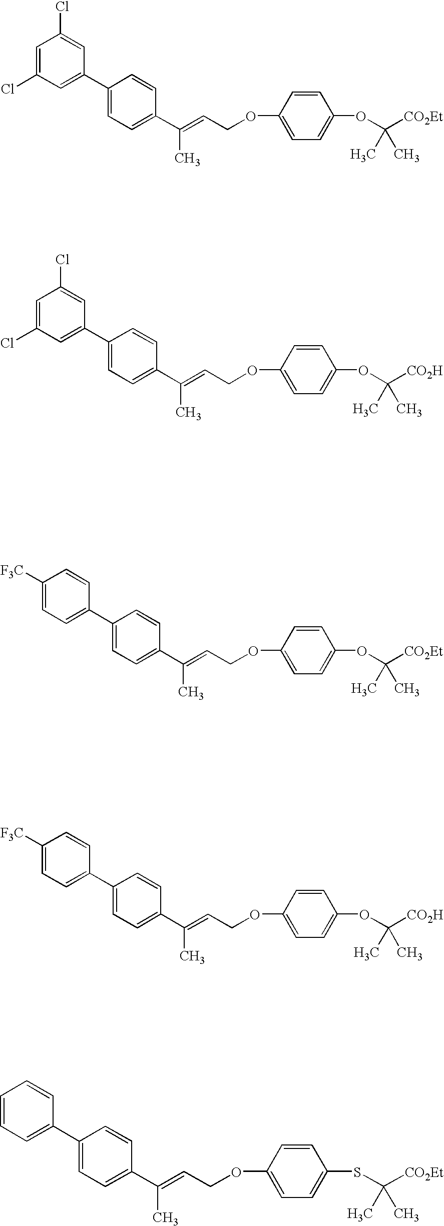 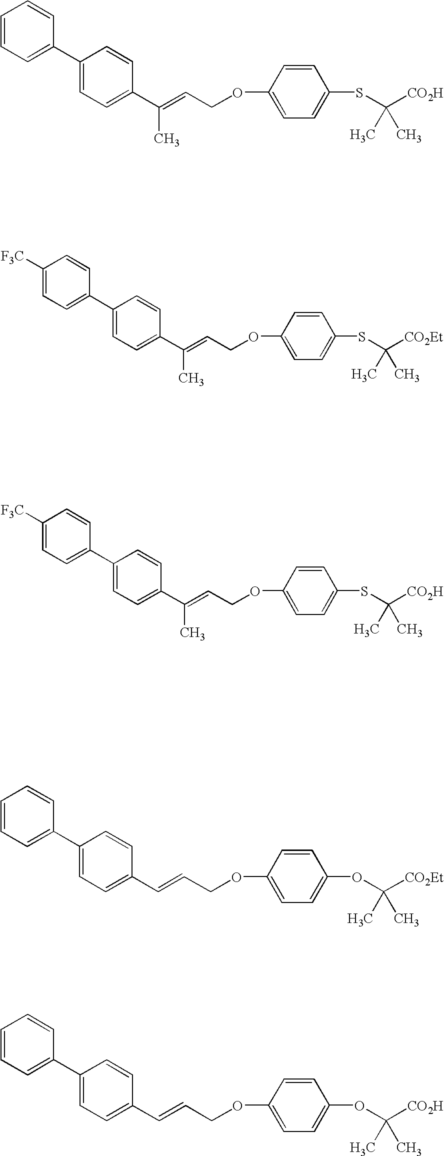 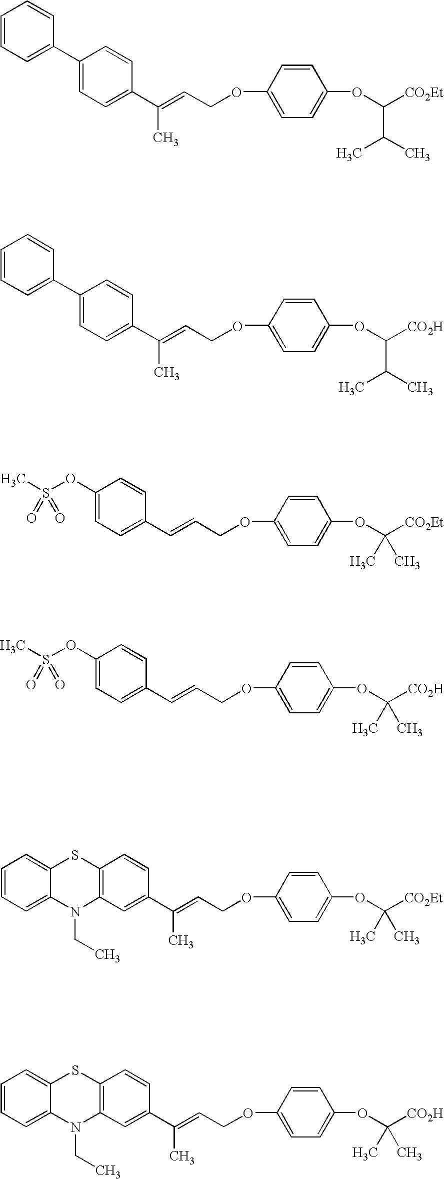 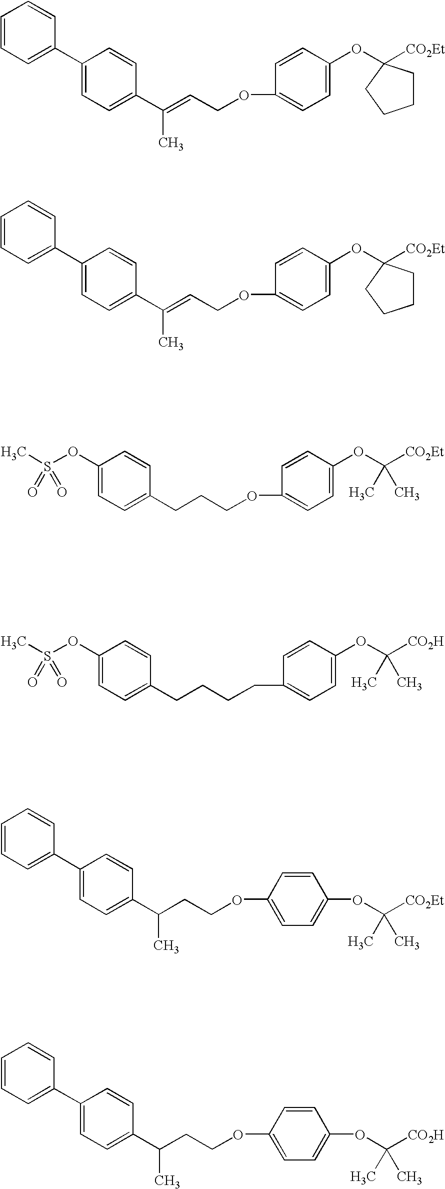
Another group of preferred compounds of the formula (I) are:

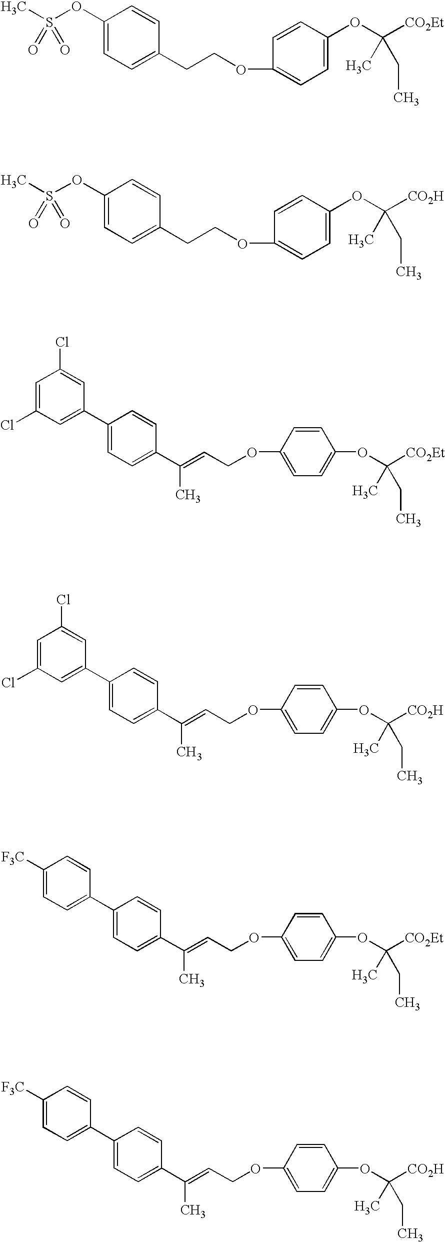

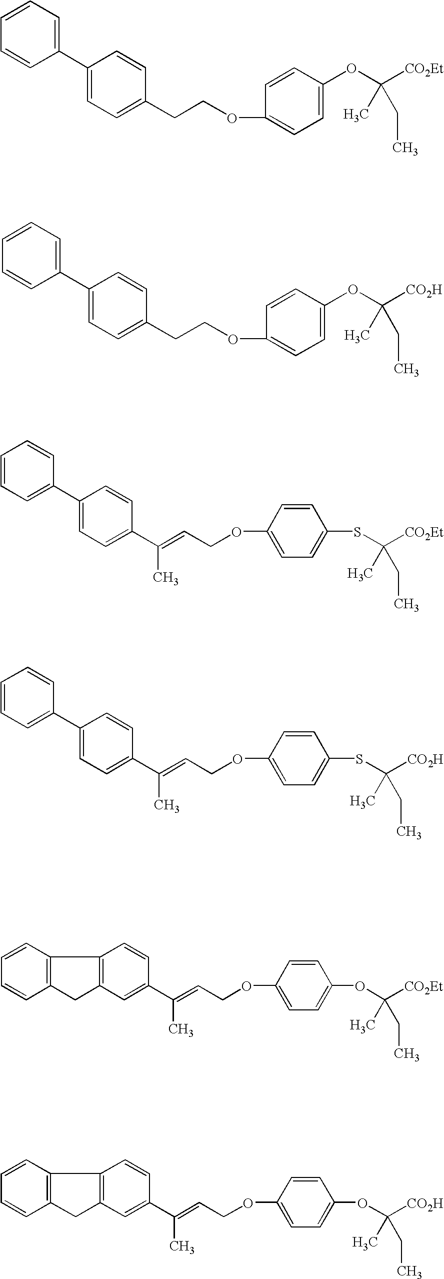

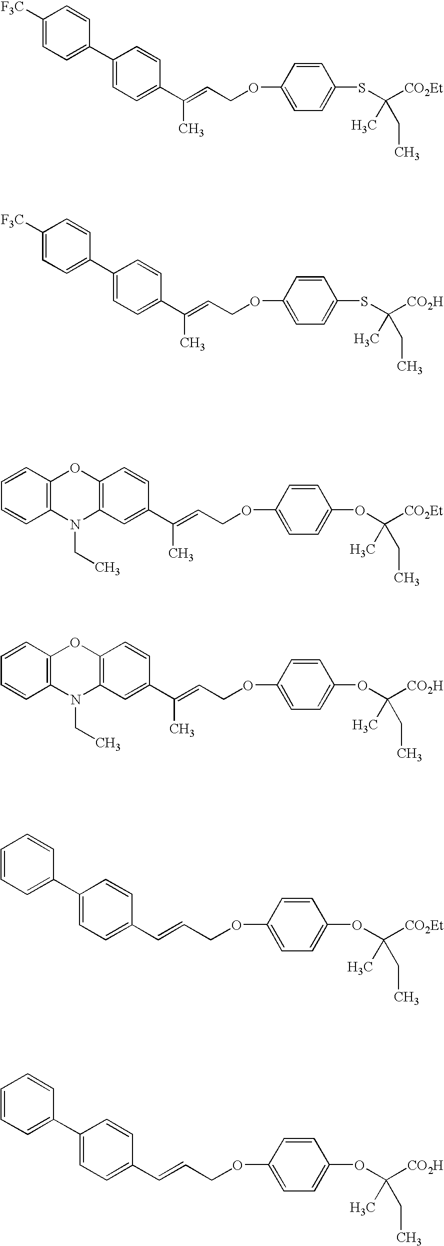

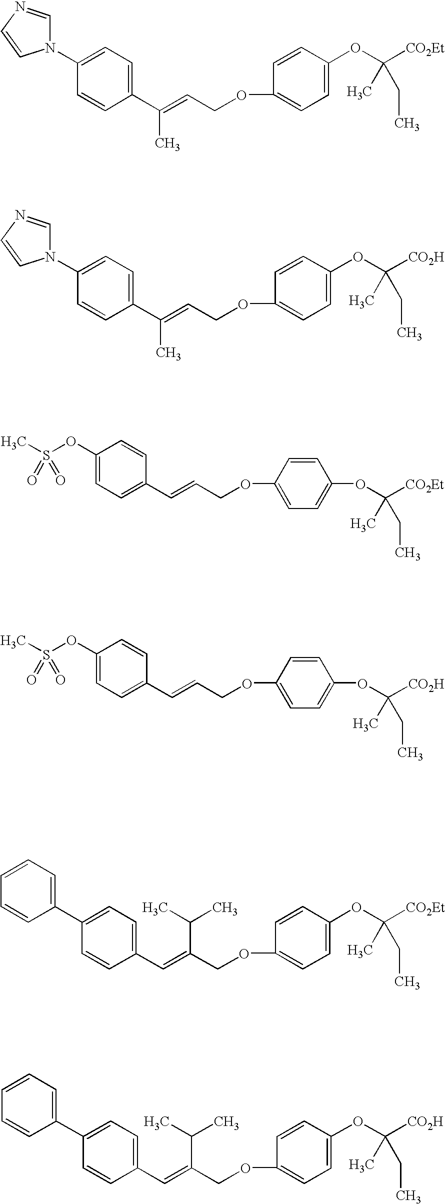

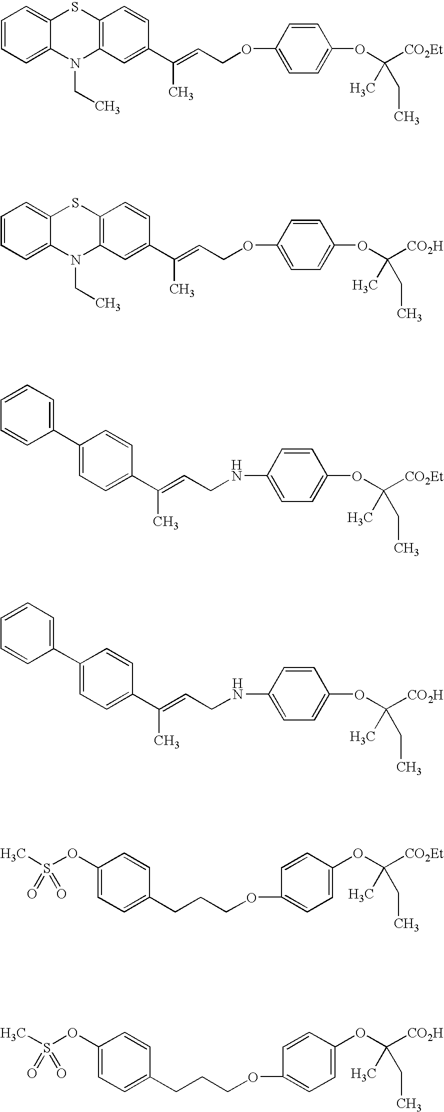

Yet another group of preferred compounds of the formula (I) are:

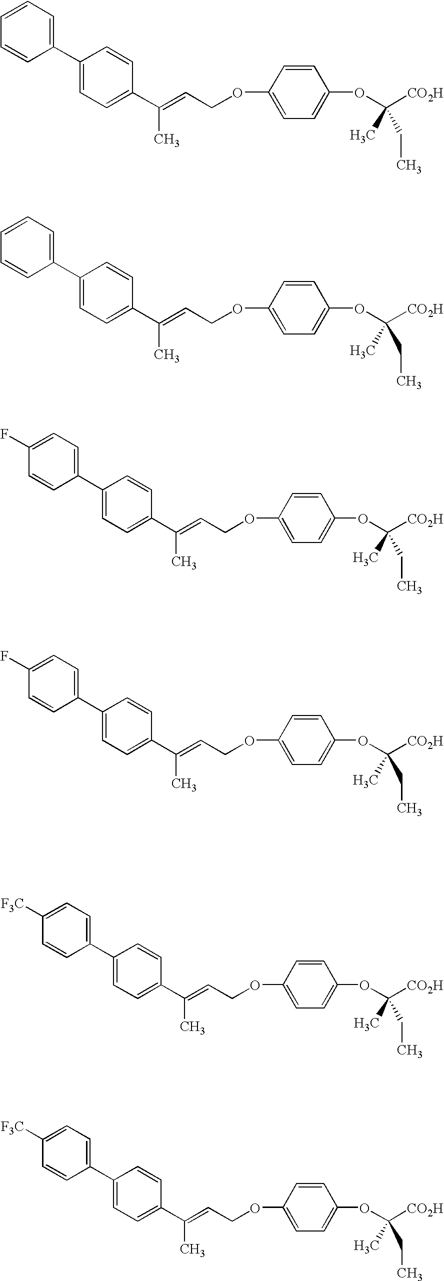

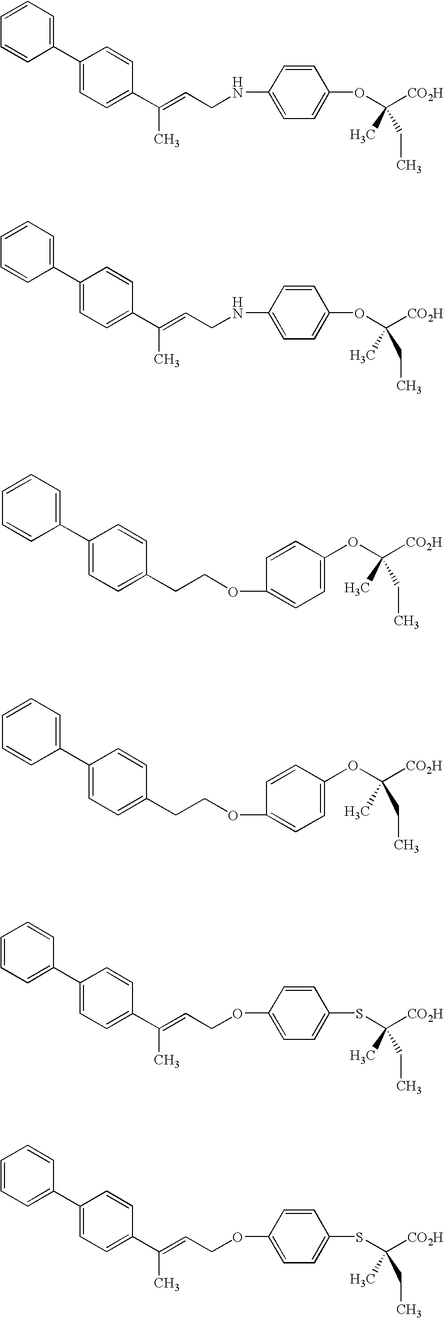

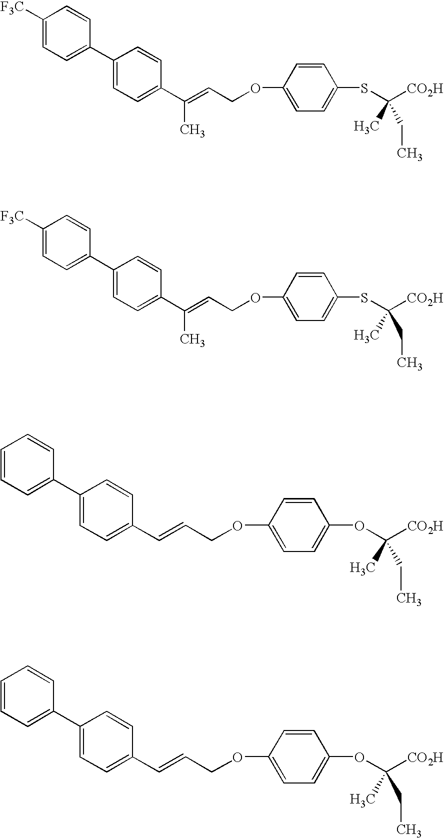

More preferred compounds of the formula (I) are:

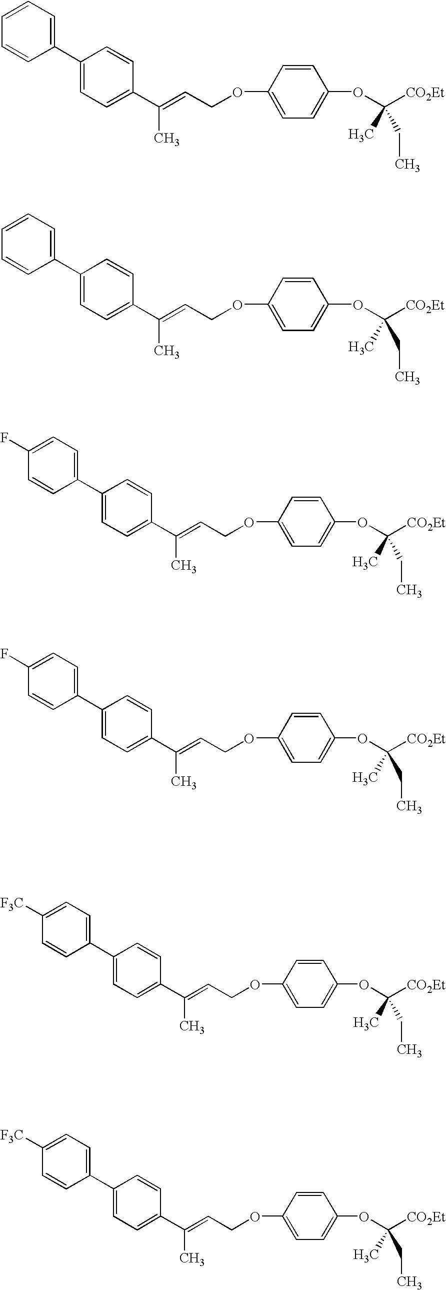

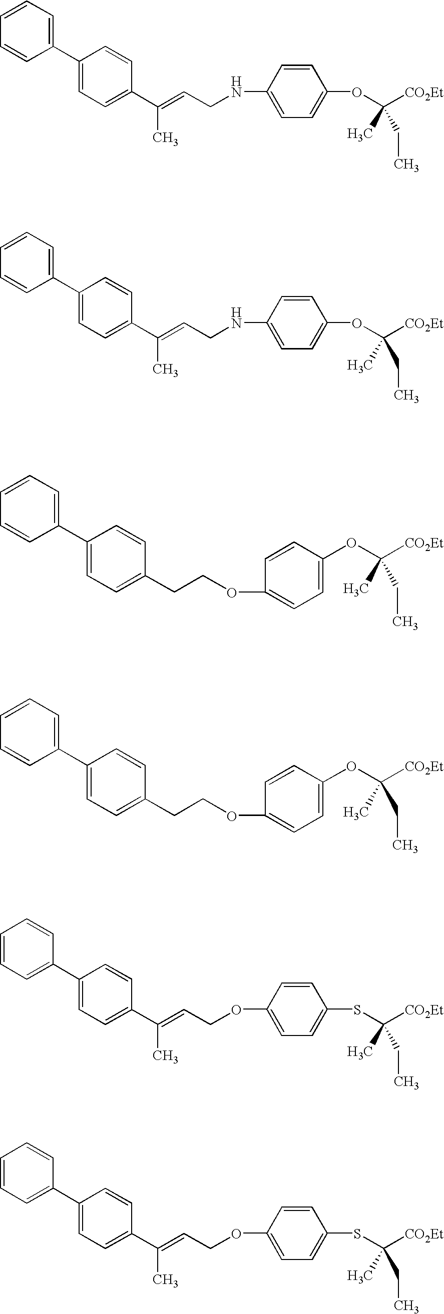

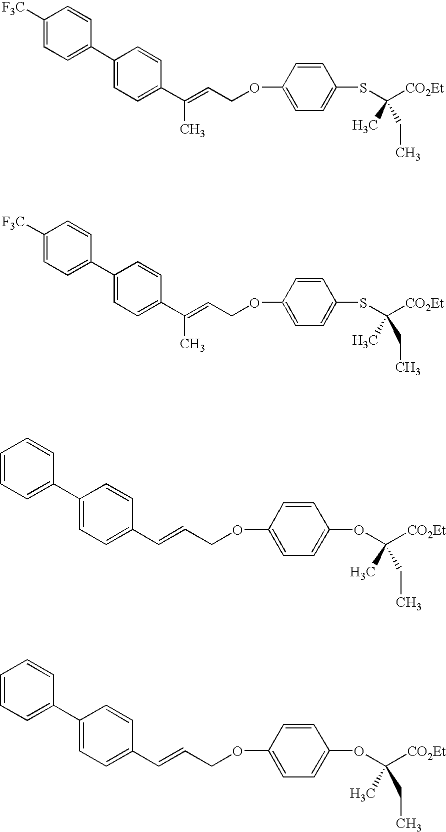

Still more preferred compounds of the formula (I) are:

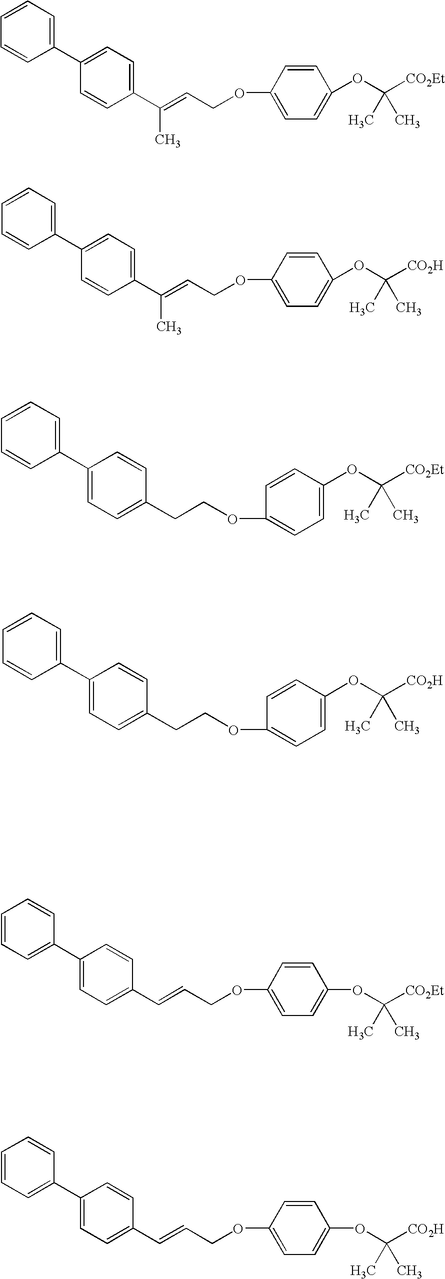

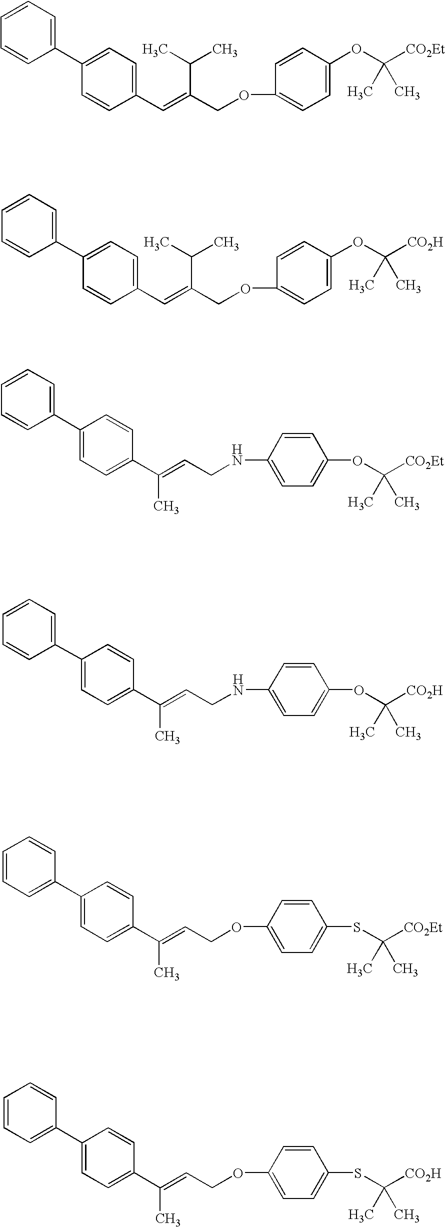

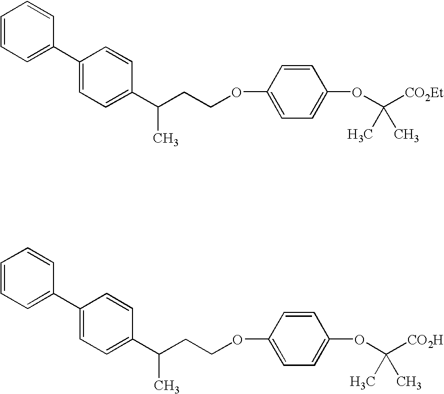

Still more preferred compounds of the formula (I) are:

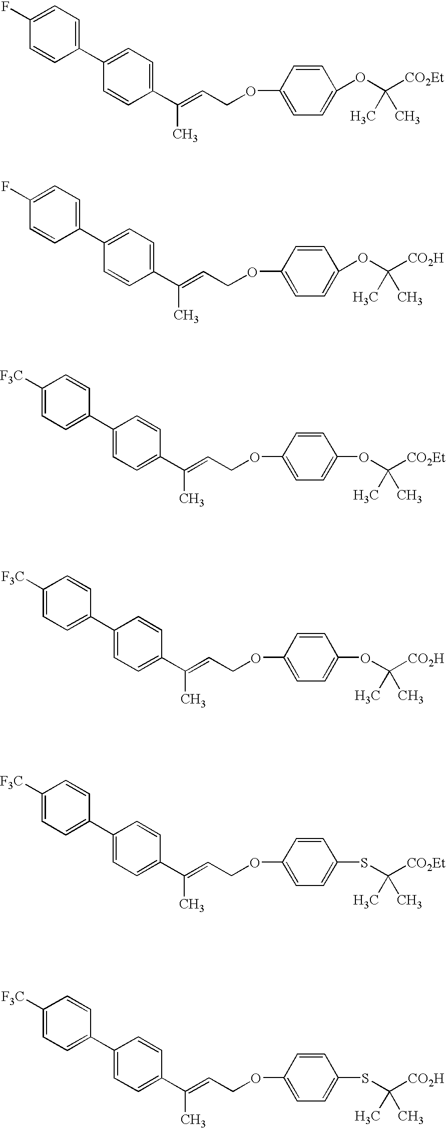

Still more preferred compounds of the formula (I) are:

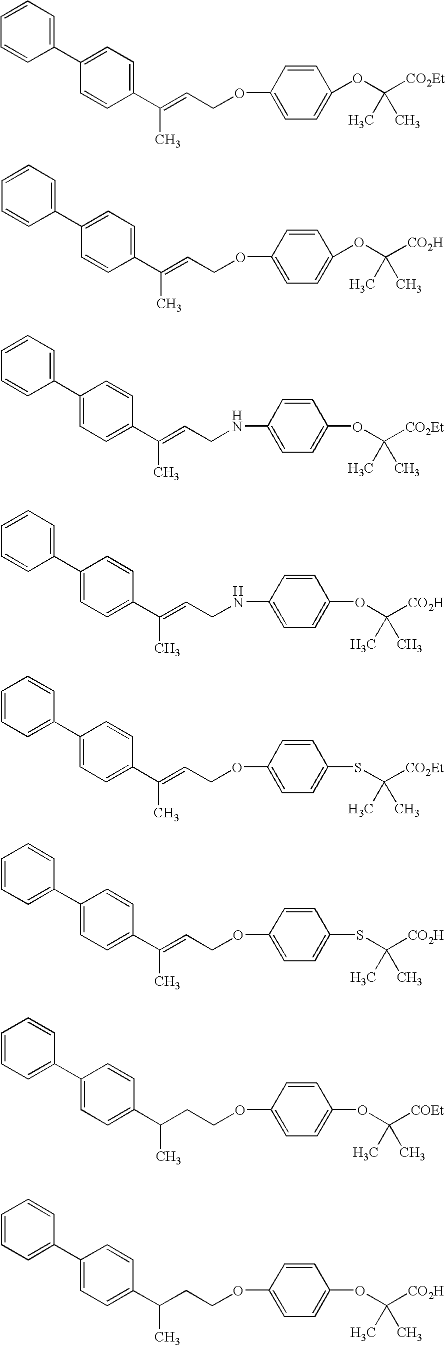

Still more preferred compounds of the formula (I) are:

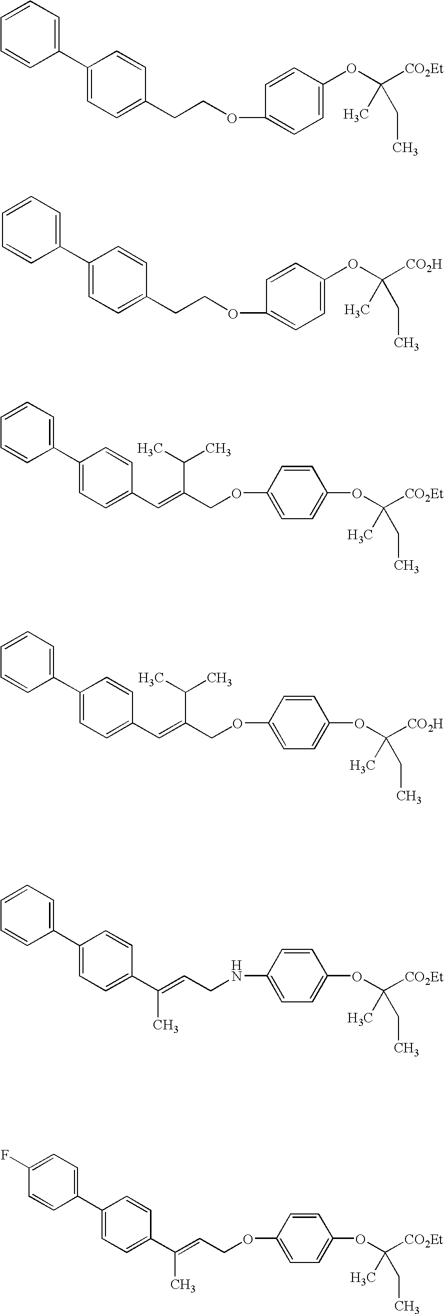

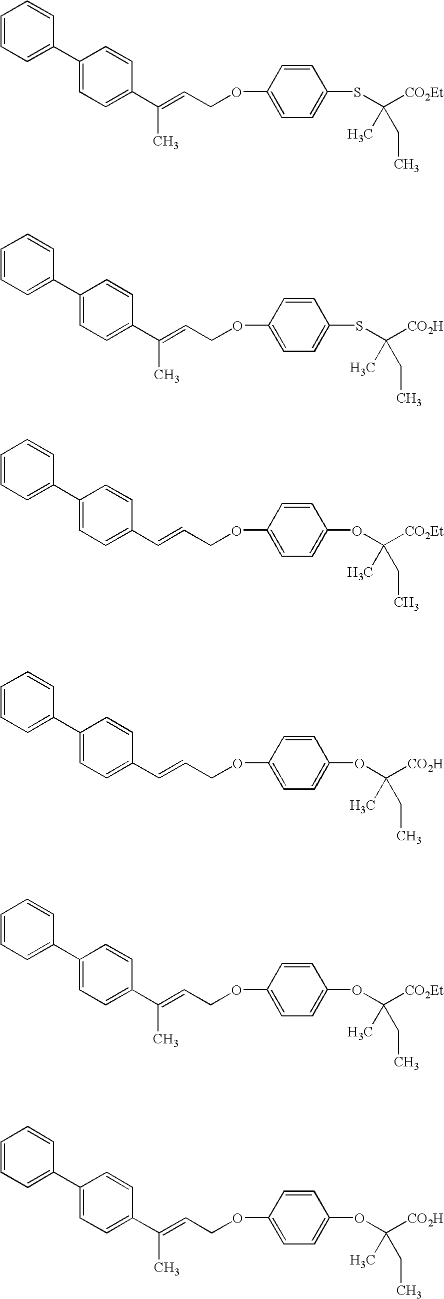

Still more preferred compounds of the formula (I) are:

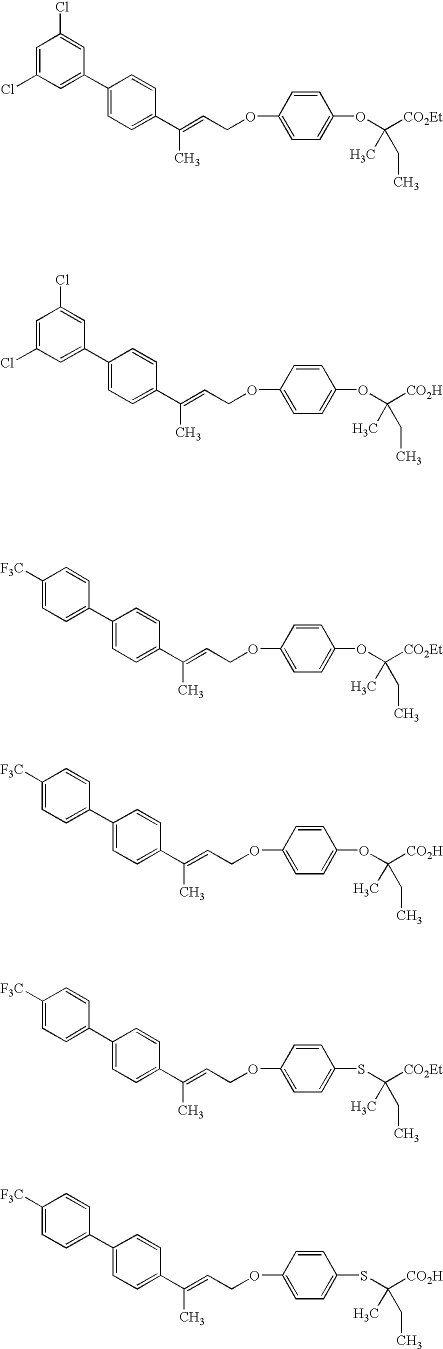

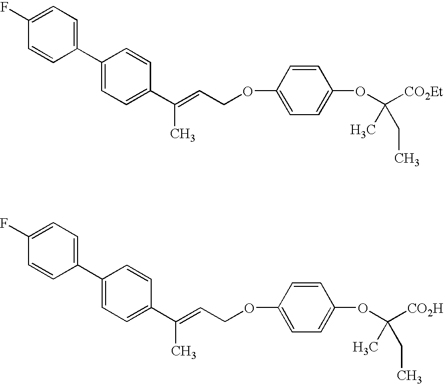

Still more preferred compounds of the formula (I) are:

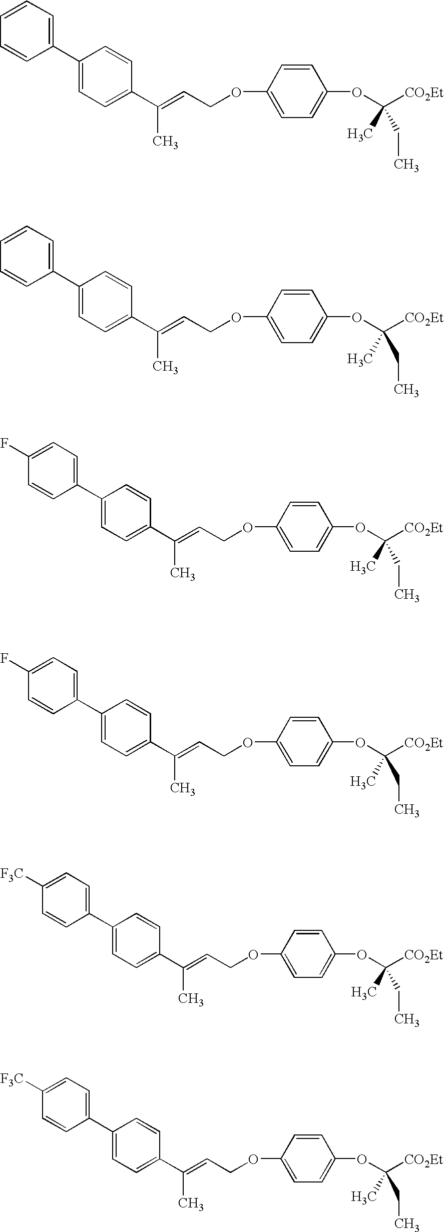

Still more preferred compounds of the formula (I) are:

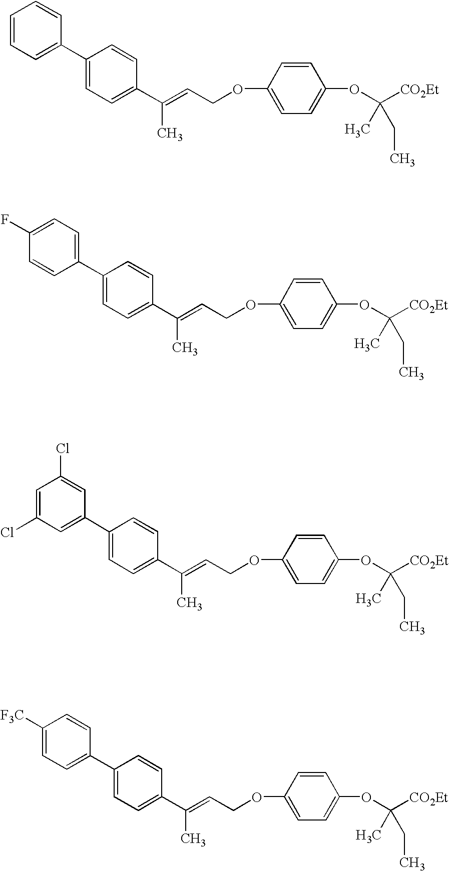

Still more preferred compounds of the formula (I) are:

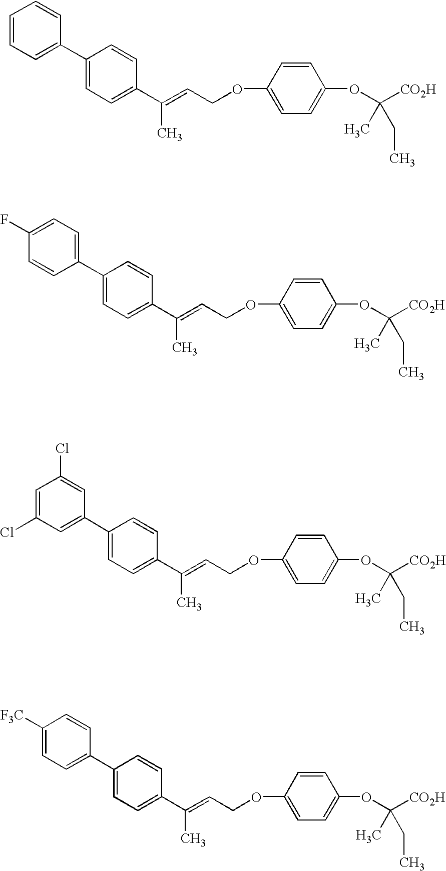

Still more preferred compounds of the formula (I) are:

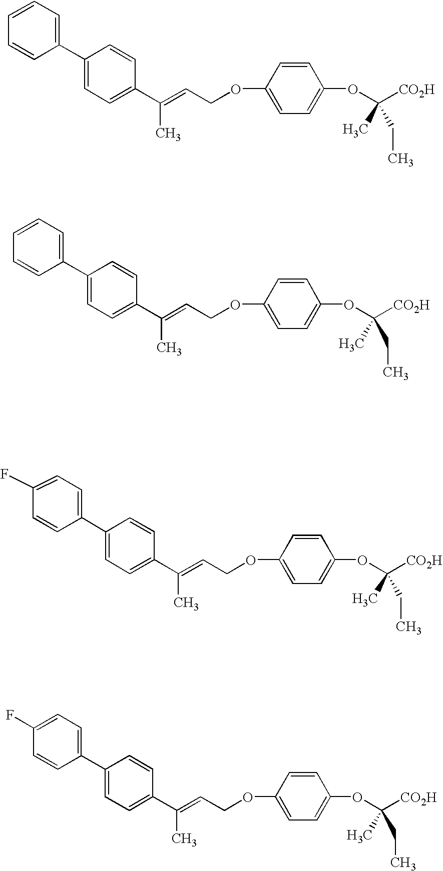

Essentially preferred compound of the present invention is

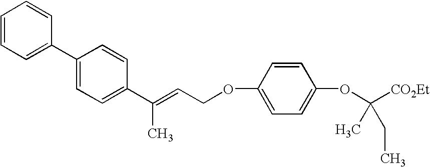

Another essentially preferred compound of the present invention is

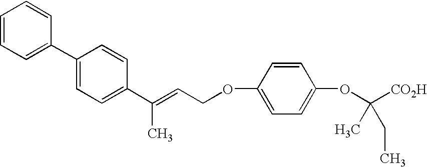

Another essentially preferred compound of the present invention is

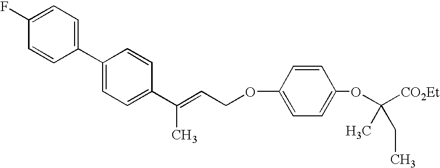

Another essentially preferred compound of the present invention is

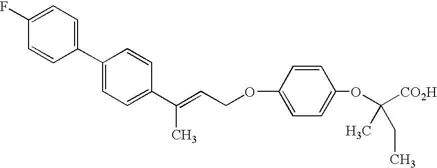

Another essentially preferred compound of the present invention is

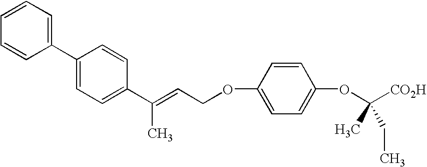

Another essentially preferred compound of the present invention is

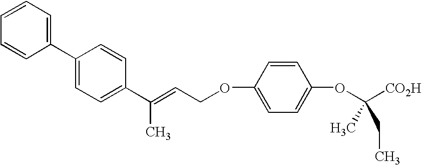

Another essentially preferred compound of the present invention is

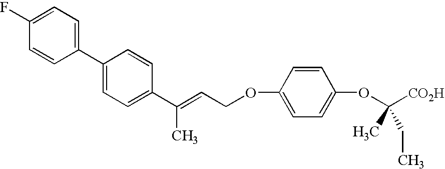

Another essentially preferred compound of the present invention is

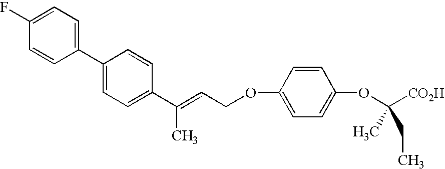

Another essentially preferred compound of the present invention is

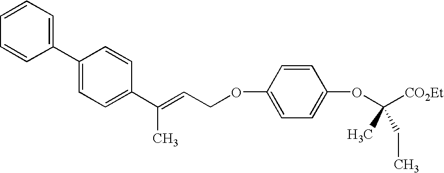

Yet another essentially preferred compound of the present invention is

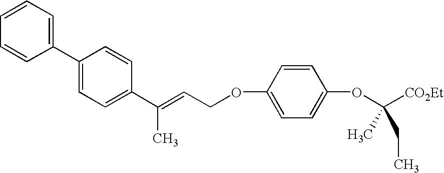

Yet another essentially preferred compound of the present invention is

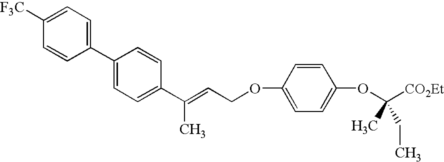

Yet another essentially preferred compound of the present invention is

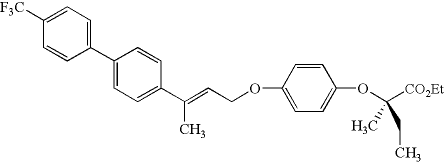

The novel compounds of the general formula (I), as defined above, have PPAR agonist activity for reducing lipid levels, lowering cholesterol and reducing body weight and reducing blood glucose with beneficial effects in the treatment and/or prophylaxis of diseases related to increased levels of lipids, atherosclerosis, coronary artery diseases, Syndrome-X, impaired glucose tolerance, insulin resistance, insulin resistance leading to type 2 diabetes and diabetic complications thereof;

The compounds of the present invention are administered in dosages effective to agonize peroxisome proliferators activated receptor where such treatment is needed, as, for example, in the prevention or treatment of diabetes, hypertension, coronary heart disease, atherosclerosis, stroke, peripheral vascular diseases, psoriasis, polycystic ovarian syndrome (PCOS), inflammatory bowel diseases, osteoporosis, myotonic dystrophy, pancreatitis, retinopathy, arteriosclerosis, xanthoma and related disorders. For use in medicine, the salts of the compounds of this invention refer to non-toxic “pharmaceutically acceptable salts.” Other salts may, however, be useful in the preparation of the compounds according to the invention or of their pharmaceutically acceptable salts.

Salts encompassed within the term “pharmaceutically acceptable salts” refer to non-toxic salts of the compounds of this invention which are generally prepared by reacting the free acid with a suitable organic or inorganic base. Representative salts include the following:

Li, Na, K, Ca, Mg, Fe, Cu, Zn, Mn; N,N′-diacetylethylenediamine, betaine, caffeine, 2-diethylaminoethanol, 2-dimethylaminoethanol, N-ethylmorpholine, N-ethylpiperidine, glucamine, glucosamine, hydrabamine, isopropylamine, methylglucamine, morpholine, piperazine, piperidine, procaine, purines, theobromine, triethylamine, trimethylamine, tripropylamine, tromethamine, diethanolamine, meglumine, ethylenediamine, N,N′-diphenylethylenediamine, N,N′-dibenzylethylenediamine, N-benzyl phenylethylamine, choline, choline hydroxide, dicyclohexylamine, metformin, benzylamine, phenylethylamine, dialkylamine, trialkylamine, thiamine, aminopyrimidine, aminopyridine, purine, spermidine; alkylphenylamine, glycinol, phenyl glycinol; glycine, alanine, valine, leucine, isoleucine, norleucine, tyrosine, cystine, cysteine, methionine, proline, hydroxy proline, histidine, ornithine, lysine, arginine, serine, threonine, phenylalanine; unnatural amino acids; D-isomers or substituted amino acids; guanidine, substituted guanidine wherein the substituents are selected from nitro, amino, alkyl, alkenyl, alkynyl, ammonium or substituted ammonium salts and aluminum salts; sulphates, nitrates, phosphates, perchlorates, borates, hydrohalides, acetates, tartrates, maleates, citrates, succinates, palmoates, methanesulphonates, benzoates, salicylates, hydroxynaphthoates, benzenesulfonates, ascorbates, glycerophosphates, or ketoglutarates.

The compounds of the present invention, may have chiral centers and occur as racemates, racemic mixtures and as individual diastereomers, or enantiomers.

The present invention includes within its scope prodrugs of the compounds of this invention. In general, such pro drugs will be functional derivatives of the compounds of this invention which are readily convertible in vivo into the required compound. Thus, in the methods of treatment of the present invention, the term “administering” shall encompass the treatment of the various conditions described with the compound specifically disclosed or with a compound which may not be specifically disclosed, but which converts to the specified compound in vivo after adminmstration to the patient. Conventional procedures for the selection and preparation of suitable prodrug derivatives are described, for example, in “Design of Prodrugs,” ed. H. Bundgaard, Elsevier, 1985. Metabolites of these compounds include active species produced upon introduction of compounds of this invention into the biological milieu.

The stereoisomers of the present invention include enatiomers and/or geometrical isomers such as (R), (S), a mixture of (R) and (S), (E), (Z) or a mixture of (E) and (Z) or combinations thereof such as (S)(E), (S)(Z), (R)(E), (R)(Z) and the like. The individual optical isomers or required isomers may be obtained by using reagents in such a way to obtain single isomeric form in the process wherever applicable or by conducting the reaction in the presence of reagents or catalysts in their single enantiomeric form. Some of the preferred methods of resolution of racemic compounds include use of microbial resolution, resolving the diastereomeric salts formed with chiral acids such as mandelic acid, camphorsulfonic acid, tartaric acid, lactic acid, and the like wherever applicable or chiral bases such as brucine, cinchona alkaloids and their derivatives and the like. Commonly used methods are compiled by Jaques et al in “Enantiomers, Racemates and Resolution” (Wiley Interscience, 1981). Where appropriate the compounds of formula (I) may be resolved by treating with chiral amines, aminoacids, aminoalcohols derived from aminoacids; conventional reaction conditions may be employed to convert acid into an amide; the diastereomers may be separated either by fractional crystallization or chromatography and the stereoisomers of compound of formula (I) may be prepared by hydrolyzing the pure diastereomeric amide.

The terms “individual,” “subject,” “host,” and “patient” refer to any subject for whom diagnosis, treatment, or therapy is desired. In one embodiment, the individual, subject, host, or patient is a human. Other subjects may include, but are not limited to, animals including but not limited to, cattle, sheep, horses, dogs, cats, guinea pigs, rabbits, rats, primates, opossums and mice. Other subjects include species of bacteria, phages, cell cultures, viruses, plants and other eucaryotes, prokaryotes and unclassified organisms. The terms “treatment,” “treating,” “treat,” and the like are used herein to refer generally to obtaining a desired pharmacological and/or physiological effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a subject, particularly a human, and includes: (a) preventing the disease or symptom from occurring in a subject which may be predisposed to the disease or symptom, but has not yet been diagnosed as having it; (b) inhibiting the disease symptom, i.e., arresting its development; or (c) relieving the disease symptom, i.e., causing regression of the disease or symptom.

The term “therapeutically effective amount” shall mean that amount of a drug or pharmaceutical agent that will elicit the biological or medical response of a tissue, system or patient that is being sought.

It is to be understood that this invention is not limited to the particular methodology, protocols, cell lines, constructs, and reagents described herein and as such may vary. It is also to be understood that the terminology used herein is for the purpose of describing particular embodiments only, and is not intended to limit the scope of the present invention which will be limited only by the appended claims.

The pharmaceutical composition may be in the forms normally employed, such as tablets, capsules, powders, syrups, solutions, suspensions and the like, may contain flavorants, sweeteners etc. in suitable solid or liquid carriers or diluents, or in suitable sterile media to form injectable solutions or suspensions. Such compositions typically contain from 0.1 to 50%, preferably 1 to 20% by weight of active compound, the remainder of the composition being pharmaceutically acceptable carriers, diluents or solvents.

Suitable pharmaceutically acceptable carriers include solid fillers or diluents and sterile aqueous or organic solutions. The active ingredient will be present in such pharmaceutical compositions in the amounts sufficient to provide the desired dosage in the range as described above. Thus, for oral administration, the active ingredient can be combined with a suitable solid or liquid carrier or diluent to form capsules, tablets, powders, syrups, solutions, suspensions and the like. The pharmaceutical compositions, may, if desired, contain additional components such as flavourants, sweeteners, excipients and the like. For parenteral administration, the active ingredient can be combined with sterile aqueous or organic media to form injectable solutions or suspensions. For example, solutions in sesame or peanut oil, aqueous propylene glycol and the like can be used, as well as aqueous solutions of water-soluble pharmaceutically-acceptable acid addition salts or salts with base of the compounds. Aqueous solutions with the active ingredient dissolved in polyhydroxylated castor oil may also be used for injectable solutions. The injectable solutions prepared in this manner can then be administered intravenously, intraperitoneally, subcutaneously, or intramuscularly, with intramuscular administration being preferred in humans.

For nasal administration, the preparation may contain the active ingredient of the present invention dissolved or suspended in a liquid carrier, in particular an aqueous carrier, for aerosol application. The carrier may contain additives such as solubilizing agents, such as propylene glycol, surfactants, absorption enhancers such as lecithin (phosphatidylcholine) or cyclodextrin or preservatives such as parabenes.

Tablets, dragees or capsules having talc and/or a carbohydrate carried binder or the like are particularly suitable for any oral application. Preferably, carriers for tablets, dragees or capsules include lactose, corn starch and/or potato starch. A syrup or elixir can be used in cases where a sweetened vehicle can be employed.

The dosage regimen utilizing the compounds of the present invention is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician, veterinarian or clinician can readily determine and prescribe the effective amount of the drug required to prevent, counter or arrest the progress of the condition.

Oral dosages of the present invention, when used for the indicated effects, will range between about 0.01 mg per kg of body weight per day (mg/kg/day) to about 500 mg/kg/day, preferably 0.01 to 10 mg/kg/day, and most preferably 0.1 to 5.0 mg/kg/day. For oral administration, the compositions are preferably provided in the form of tablets containing 0.01, 0.05, 0.1, 0.5, 1.0, 2.5, 5.0, 10.0, 15.0, 25.0, 50.0, 100 and 500 milligrams of the active ingredient for the symptomatic adjustment of the dosage to the patient to be treated. A medicament typically contains from about 0.01 mg to about 500 mg of the active ingredient, preferably, from about 1 mg to about 100 mg of active ingredient. Intravenously, the most preferred doses will range from about 0.1 to about 10 mg/kg/minute during a constant rate infusion. Advantageously, compounds of the present invention may be administered in a single daily dose, or the total daily dosage may be administered in divided doses of two, three or four times daily. Furthermore, preferred compounds for the present invention can be administered in intranasal form via topical use of suitable intranasal vehicles, or via transdermal routes, using those forms of transdermal skin patches well known to those of ordinary skill in the art. To be administered in the form of a transdermal delivery system, the dosage administration will, of course, be continuous rather than intermittent throughout the dosage regimen.

In the methods of the present invention, the compounds herein described in detail can form the active ingredient, and are typically administered in admixture with suitable pharmaceutical diluents, excipients or carriers (collectively referred to herein as ‘carrier’ materials) suitably selected with respect to the intended form of administration, that is, oral tablets, capsules, elixirs, syrups and the like, and consistent with conventional pharmaceutical practices.

For instance, for oral administration in the form of a tablet or capsule, the active drug component can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like; for oral administration in liquid form, the oral drug components can be combined with any oral, non-toxic, pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include starch, gelatin, natural sugars such as glucose or betalactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, soaium chloride and the like. Disintegrators include, without limitation, starch, methyl cellulose, agar, bentonite, xanthan gum and the like.

The compounds of the present invention can also be administered in the form of liposome delivery systems, such as small unilamellar vesicles, large umilamellar vesicles and multilamellar vesicles. Liposomes can be formed from a variety of phospholipids, such as cholesterol, stearylamine or phosphatidylcholines.

Compounds of the present invention may also be delivered by the use of monoclonal antibodies as individual carriers to which the compound molecules are coupled. The compounds of the present intention may also be coupled with soluble polymers as targetable drug carriers. Such polymers can include polYVinylpyrrolidone, pyran copolYmer, polyhydioxypropylmethacrylamide-phenol, polyhydroxyethylaspartamide-phenol, or polyethyleneoxide-polylysine substituted with palmitoyl residues. Furthermore, the compounds of the present invention may be coupled to a class of biodegradable polYmers useful in achieving controlled release of a drug, for example, polylactic acid, polyglycolic acid, copolymers of polyactic and polyglycolic acid, polyepsilon caprolactone, polyhydroxy butyric acid, polyorthoesters, polyacetals, polydihydropyrans, polycyanoacrylates and crosslinked or amphipathic block copolYmers of hydrogels.

The compounds of formula (I) can generally be prepared, for example in the course of a convergent synthesis, by linkage of two or more fragments which can be derived retrosynthetically from the formula (I) in the preparation of compounds of formul 1, it may be generally necessary in the course of synthesis temporarily block functional groups which could lead to undesired reactions or side reactions in a synthetic step by protective group suited to the synthesis problem and known to the person skilled in the art. The method of fragment coupling is not restricted to the following examples, but is generally applicable for synthesis of compounds of formula (I).

The novel compounds of the present invention were prepared according to the procedure of the following schemes and examples, using appropriate materials and are further exemplified by the following specific examples. The most preferred compounds of the invention are any or all of those specifically set forth in these examples. These compounds are not, however, to be construed as forming the only genus that is considered as the invention, and any combination of the compounds or their moieties may itself form a genus. The following examples further illustrate details for the preparation of the compounds of the present invention. Those skilled in the art will readily understand that known variations of the conditions and processes of the following preparative procedures can be used to prepare these compounds. All temperatures are degrees Celsius unless otherwise noted.

The following Schemes and Examples describe procedures for making representative compounds of the present invention. Moreover, by utilizing the procedures described in detail, one of ordinary skill in the art can readily prepare additional compounds of the present invention claimed herein. Scheme 1: The compounds of general formula (I), where p represents I and all other symbols are as defined earlier, may be prepared by the process as shown in Scheme-I below:

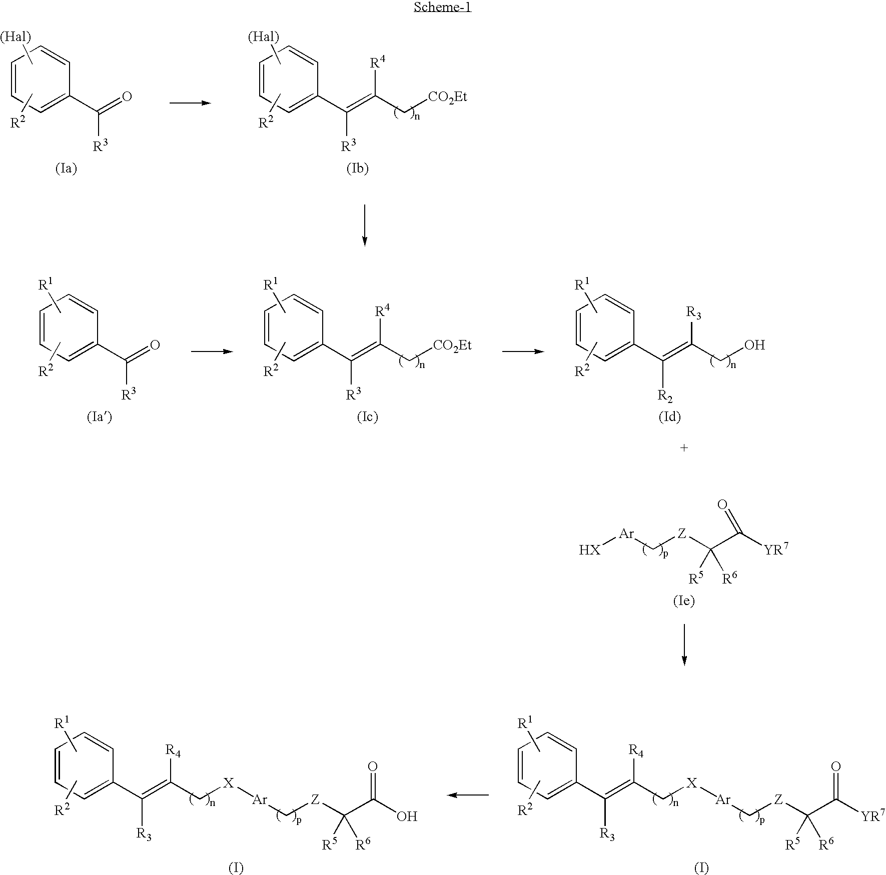

The compound of formula (Ia) is converted to a compound of formula (Ib) where ‘Hal’ represents halogen atom such as bromine or iodine, and R² represents hydrogen atom, in a Witting-Horner reaction manner, by using phosphono acetate compounds selected from substituted phosphone acetate compounds such as triethyl phosphono acetates, trimethylphosphono acetate, Ph₃P⁺—CH₂⁻—CO₂Et and the like. The base used in the reaction may be selected from sodium hydride, potassium tertiary butoxide, potassium hydroxide, sodium methoxide, sodium ethoxide and the like. The solvent used in the reaction is selected from alcohol selected from methanol, ethanol, propanol, isopropanol and the like or mixtures thereof, tetrahydrofuran, ether, dioxane, dimethoxyethane and the like. The temperature of the reaction is maintained in the range of 0 to 10° C., preferably 0° C. The duration of the reaction is maintained in the range of 10 to 24 hours, preferably in the range of 12 to 18 hours.

The compound of formula (Ib), where ‘Hal’ represents halogen atom such as bromine or iodine, and R² represents hydrogen atom, is converted to a compound of formula (Ic), where R¹ represent aryl group and R² represents hydrogen atom, in a Suzuki coupling reaction manner, by using aryl boronic acid with palladium catalyst like Pd(PPh₃)₄, PdCl₂, Pd(dba)₂ (dba: dibenz[a,h]anthracene) and the like. The solvent used in the reaction is selected from terahydrofuran, dioxane, acetonitrile, dimethylether, diethylether, dimethylformamide and the like. The reaction may be carried out at a reflux temperature of the solvent used. The duration of the reaction may be in the range of 15 to 28 hours, preferably in the range of 15 to 24 hours.

The compound of formula (Ic), is prepared from compound of formula (Ia′), where R¹ and R² are as defined in the formula (I), by using substituted phosphone acetate compounds selected from triethyl phosphono acetates, trimethylphosphono acetate, Ph₃P⁺—CH₂⁻—CO₂Et and the like.

The reduction of the compound of formula (Ic) to a compound of formula (Id) may be carried out in the presence of a reducing agent selected from DIBAL-H, AlH₃, lithium aluminium (LAH) and the like. The solvent used in the reaction may be selected from toluene, tetrahydrofuran (THF), ether, dioxane, dimethoxyethane and the like. The temperature of the reaction may be in the range of −90 to −25° C., preferably in the range of −80 to −60° C. The duration of the reaction may in the range of 0.5 h to 2 hours, preferably in the range of 0.5 to 1 hours. The temperature and duration of the reaction can be decreased in the presence of AlH₃.

The coupling of a compound of formula (Id) with a compound of formula (Ie), where p represents 1, Y represents O or S, (Mitsinobu reaction) to obtain a compound of formula (I), where p represents 1, Y represents O or S, R⁷ represents all the groups as defined earlier, except hydrogen atom and all other symbols are as defined earlier, by using PPh₃, diisopropyl azadicarboxylate (DIAD), diethyl azadicarboxylate (DEAD) and the like. The solvent used in the reaction is selected from tetrahydrofuran, toluene, benzene and the like. The reaction temperature may be in the range of 20 to 40° C., preferably at room temperature. The duration of the reaction may be in the range of 40 to 80 hours, preferably in the range of 40 to 72 hours.

The compound of general formula (I) where R⁷ represents hydrogen atom, Y represents O or S, p represents 1 and all other symbols are as defined earlier, may be prepared from a compound of formula (I) where R⁷ represents all groups defined earlier except hydrogen, Y represents O or S, p represents 1 and all other symbols are as defined earlier, by hydrolysis using conventional methods. The reaction may be carried out in the presence of a base such as sodium hydroxide, potassium hydroxide, lithium hydroxide, potassium carbonate, sodium carbonate and the like. The solvent used may be selected from alcohols such as methanol, ethanol, propanol, isopropanol and the like or mixtures thereof, water, tetrahydrofuran, dioxane, ether and the like or mixtures thereof. The temperature of the reaction may be in the range of 30 to 80° C., preferably at room temperature. The duration of the reaction may be in the range of 2 to 24 hours, preferably 2 to 12 hours.

The compound of general formula (I) where Z represents O or S, p represents 1 and R⁷ represents hydrogen or lower alkyl group may be converted to compound of formula (I), where Y represents NR¹¹ by reacting with appropriate amines of the formula NHR⁷R¹¹, where R⁷ and R¹¹ are as defined earlier to yield a compound of formula (I) where Y represents NR¹¹ and all other symbols are as defined earlier. Alternatively, the compound of formula (I) where YR⁷ represents OH may be converted to acid halide, preferably YR⁷═Cl, by reacting with appropriate reagents such as oxalyl chloride, thionyl chloride and the like, followed by treatment with amines of the formula NHR⁷R¹¹ where R⁷ and R¹¹ are as defined earlier. Alternatively, mixed anhydrides may be prepared from compound of formula (I) where YR⁷ represents OH and all other symbols are as defined earlier by treating with acid halides such acetyl chloride, acetyl bromide, pivaloyl chloride, dichlorobenzoyl chloride and the like. The reaction may be carried out in the presence of pyridine, triethylamine, diisopropyl ethylamine and the like. Coupling reagent such as dicyclohexylcarbodiimide/4-dimethylaminopyridine (DCC/DMAP), dicyclohexylcarbodiimide/1-hydroxybenzotriazole (DCC/HOBt), 1-ethyl-3-(3-dimethylaminopropy)carbodiimide/1-hydroxybenzotriazole (EDCI/HOBt), 2-dimethylaminoisopropyl chloride hydrochloride/1-hydroxybenzotriazole (DIC/HOBt), ethylchloroformate, isobutylchloroformate can also be used to activate the acid. The reaction may be carried out in the presence of a solvent such as halogenated hydrocarbon like chloroform (CHCl₃) or dichloromethane (CH₂Cl₂); hydrocarbon such as benzene, toluene, xylene and the like. The reaction may be carried out at a temperature in the range of −40 to 40° C., preferably at a temperature in the range of 0 to 20° C. The acid halide or mixed anhydride or activated acid obtained by coupling reagents described above thus prepared may further be treated with appropriate amine of the formula NHR⁷R¹¹ where R⁷ and R¹¹ are as defined earlier, to yield a compound of formula (I) where Y represents NR¹¹ and all other symbols are as defined earlier.
Scheme 2: The compounds of general formula (I), where p represents 1 and all other symbols are as defined earlier, may be prepared by the process as shown in Scheme-2: 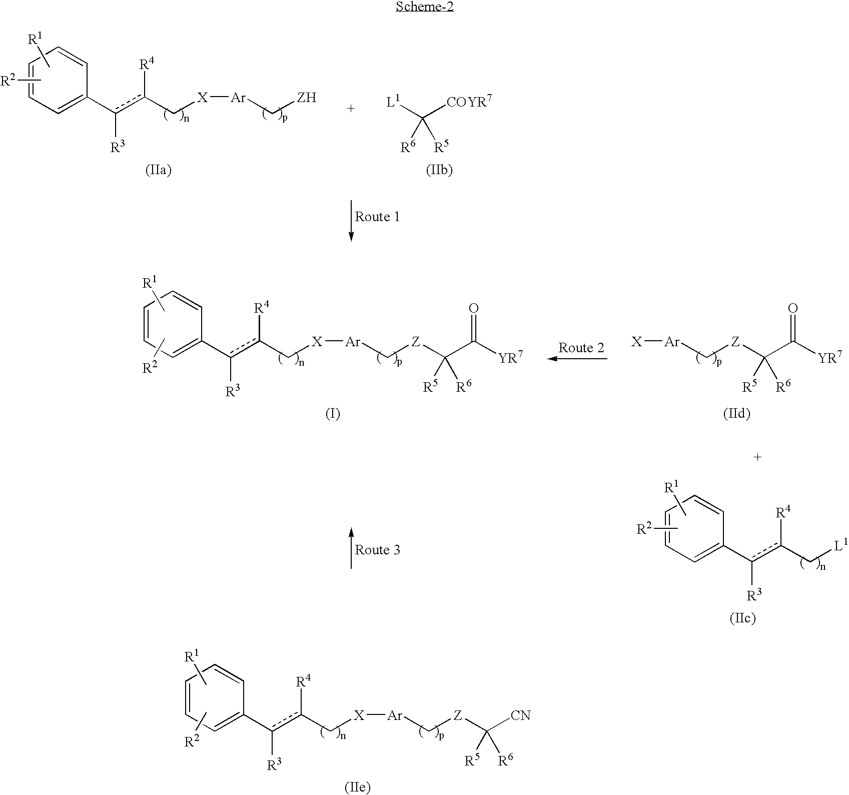
Route 1: The reaction of compound of formula (IIa) with compound of formula (IIb) where L¹ is a leaving group such as hydroxy, halogen atom, p-toluenesulfonate, methanesulfonate, trifluoromethanesulfonate and the like, and where all symbols are as defined earlier, may be carried out in the presence of an aprotic solvent such as tetrahydrofuran (THF), dimethylformamide (DNM), dimethylsulfoxide (DMSO), ethyleneglycol dimethylether (DME), toluene, benzene, xylene and the like or mixtures thereof. The reaction may be carried out in the presence of a organic base such as triethylamine, collidine, lutidine and the like or mixtures thereof. The reaction may be carried out in an inert atmosphere that may be maintained by using an inert gas such as nitrogen, helium or argon. The reaction may be effected in the presence of a base such as potassium carbonate (K₂CO₃), sodiumcarbonate (Na₂CO₃), sodamide (NaNH₂), n-BuLi, sodiumnhydride NaH), potassium hydride (KH) and the like. The reaction temperature may range from 0 to 120° C., preferably in the range of 25 to 100° C. The duration of the reaction may range from 1 to 72 hours, preferably from 2 to 24 hours.
Route 2: The reaction of compound of formula (IIc) with compound of formula (IId), where L¹ represents a leaving group such as hydroxy, halogen atom, p-toluenesulfonate, methanesulfonate, trifluoromethanesulfonate and the like, and all other symbols are as defined earlier, may be carried out in the presence of an aprotic solvent such as THF, DMF, DMSO, DME and the like or mixtures thereof. The reaction may be carried out in an inert atmosphere that may be maintained by using an inert gas such as nitrogen, argon, helium and the like. The reaction may be effected in the presence of a base such as potassium carbonate (K₂CO₃), sodium carbonate (Na₂CO₃) or sodiumhydride (NaH), potassiumhydride (KH), triethyl amine and the like or mixtures thereof. The reaction temperature may range from 0 to 120° C., preferably in the range of 25 to 100° C. The duration of the reaction may range from 1 to 72 hours, preferably from 2 to 24 hours.
Route 3: The conversion of compound of formula (IIe) to a compound of formula (I), where all symbols are as defined earlier, may be carried out either in the presence of a base or an acid and the selection of a base or an acid is not critical. Any base normally used for hydrolysis of nitrile to an acid may be employed, metal hydroxide such as sodiumhydroxide (NaOH) or potassiumhydroxide (KOH) in an aqueous solvent or any acid normally used for hydrolysis of nitrile to ester may be employed such as dry HCl in an excess of alcohol such as methanol, ethanol, propanol, isopropanol and the like. The reaction may be carried out at a temperature in the range of 0° C. to reflux temperature of the solvent used, preferably at a temperature in the range of 25° C. to reflux temperature of the solvent-used. The duration of the reaction may range from 0.25 to 48 hours.
The compound of general formula (I) where R⁷ represents hydrogen atom may be prepared by hydrolysis using conventional methods, a compound of formula (I) where R⁷ represents all groups defined earlier except hydrogen. The hydrolysis may be carried out in the presence of a base such as Na₂CO₃, K₂CO₃, NaOH, KOH, lithiumhydroxide (LiOH) and the like and a suitable solvent such as methanol, ethanol, propanol, isopropoanol, water and the like or mixtures thereof. The reaction may be carried out at a temperature in the range of 20 to 120° C. The reaction time may range from 2 to 48 hours, preferably from 2 to 12 hours.

The compound of general formula (I) where Z represents oxygen and R⁷ represents hydrogen or lower alkyl group may be converted to compound of formula (I), where Y represents NR¹¹ by reacting with appropriate amines of the formula NHR⁷R¹, where R⁷ and R¹¹ are as defined earlier to yield a compound of formula (I) where Y represents NR¹¹ and all other symbols are as defined earlier. Alternatively, the compound of formula (I) where YR⁷ represents OH may be converted to acid halide, preferably YR⁷═Cl, by reacting with appropriate reagents such as oxalyl chloride, thionyl chloride and the like, followed by treatment with amines of the formula NHR⁷R¹ where R⁷ and R¹¹ are as defined earlier. Alternatively, mixed anhydrides may be prepared from compound of formula (I) where YR⁷ represents OH and all other symbols are as defined earlier by treating with acid halides such acetyl chloride, acetyl bromide, pivaloyl chloride; dichlorobenzoyl chloride and the like. The reaction may be carried out in the presence of pyridine, triethylamine, diisopropyl ethylamine and the like. Coupling reagent such as DCC/DMAP DCC/HOBt, EDCI/HOBt, DIC/HOBt, ethylchloroformate, isobutylchloroformate can also be used to activate the acid. The reaction may be carried out in the presence of a solvent such as halogenated hydrocarbon like CHCl₃ or CH₂Cl₂; hydrocarbon such as benzene, toluene, xylene and the like. The reaction may be carried out at a temperature in the range of 40 to 40° C., preferably at a temperature in the range of 0 to 20° C. The acid halide or mixed anhydride or activated acid obtained by coupling reagents described above thus prepared may further be treated with appropriate amine of the formula NHR⁷R¹ where R⁷ and R¹¹ are as defined earlier, to yield a compound of formula (I) where Y represents NR¹¹ and all other symbols are as defined earlier.

Scheme 3: The compounds of general formula (I), where p represents 2-6 and all other symbols are as defined earlier may be prepared by the process as shown in Scheme-3 below:

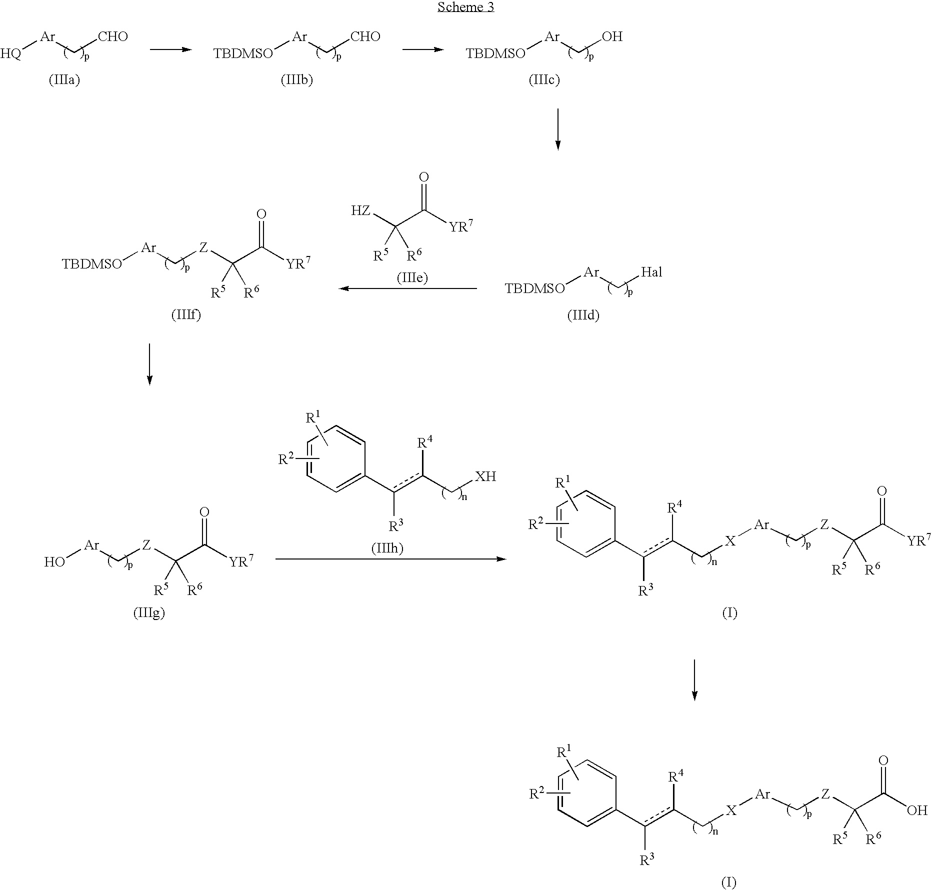

The compound of formula (IIIa) is converted to a compound of formula (IIIb) by reacting with TBDMS-Hal, where ‘Hal’ represents halogen atom. (CH₃)₃Si-Hal, Ph₃C-Hal may also be used. The base used in the reaction may be selected from triethylamine, Na₂CO₃, K₂CO₃ and the like. The solvent used in the reaction may be selected from dichloromethane, tetrahydrofuran, chloroform, dimethylether, diethylether, dioxane, benzene, toluene or mixtures thereof. The temperature of the reaction may be in the range of 0° C. to room temperature. The duration of the reaction may from 8 to 20 hours, preferably 8 to 12 hours.

The compound of formula (IIIb) is converted to a compound of formula (IIId) by using sodium borohydrate (NaBH₄). The reaction may be carried out in the presence of an alcohol such as methanol, ethanol, proanol, isopropanol and the like. The reaction may be carried out at room temperature for a duration in the range of 1 to 4 hours, preferably 1 to 2 hours.

The compound of formula (IIIc) is converted to a compound of formula (IIId) in the presence of C(Hal)₄, where ‘Hal’ represents halogen atom. The reaction may be carried out in the presence of PPh₃. The solvent used in the reaction may be selected from dichloromethane, tetrahydrofuran, chloroform, dimethylether, diethylether, dioxane, benzene, toluene or mixtures thereof. The reaction may be carried out at room temperature. The duration of the reaction may be in the range of 0.5 to 2 hours, preferably 0.5 to 1 hours.

The compound of formula (IIId) is reacted with the compound of formula (Me) to obtain a compound of formula (IIIf). The reaction may be carried out in the presence of a base such as NaH, KH, sodium amide, potassium tertiary butoxide etc. The solvent used in the reaction may be selected from DMSO, THF, toluene, benzene and the like or mixtures thereof. The duration of the reaction may be in the range of 50 to 90° C., preferably in the range of 60 to 80° C. The duration of the reaction may vary in the range of 8 to 15 hours, preferably 8 to 12 hours.

The deprotection of compound of formula (IIIf) to obtain a compound of formula (IIIg) may be carried out by using tetrabutylammoniumfluoride (TBAF). The reaction may be carried in the presence of suitable solvent such as water, THF, dioxane, dichloromethane, chloroform, methanol, ethanol etc. or mixtures thereof. The reaction may be carried out at a temperature in the range of 20 to 40° C., preferably at room temperature. The reaction time may range from 1 to 6 hours, preferably from 1 to 4 hours.

The compound of formula (IIIg) is converted to a compound of formula (I), where Y represents O or S, R⁷ represents all groups as defined earlier but not hydrogen. The reaction may be carried out by using triphenylphosphine (PPh₃), DIAD, DEAD and the like. The solvent used in the reaction is selected from tetrahydrofuran, toluene, benzene and the like. The reaction temperature may be in the range of 20 to 40° C., preferably at room temperature. The duration of the reaction may be in the range of 40 to 80 hours, preferably in the range of 40 to 72 hours.

The compound of general formula (I) where R⁷ represents hydrogen atom, Y represents O or S, p represents I and all other symbols are as defined earlier, may be prepared from a compound of formula (I) where R⁷ represents all groups defined earlier except hydrogen, Y represents O or S, p represents 1 and all other symbols are as defined earlier, by hydrolysis using conventional methods. The reaction may be carried out in the presence of a base such as sodium hydroxide, potassium hydroxide, lithium hydroxide, potassium carbonate, sodium carbonate and the like. The solvent used may be selected from alcohols such as methanol, ethanol, propanol, isopropanol and the like or mixtures thereof, water, tetrahydrofuran, dioxane, ether and the like or mixtures thereof. The temperature of the reaction may be in the range of 30 to 80° C., preferably at room temperature. The duration of the reaction may be in the range of 2 to 24 hours, preferably 2 to 12 hours.

The compounds of formula (I) may be resolved further into (Ii) and (Iii), 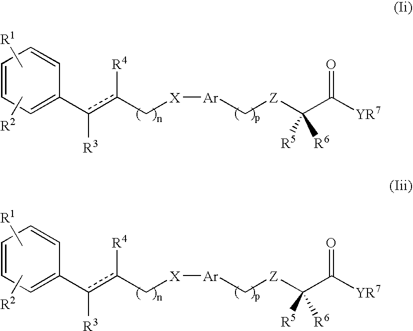
where all symbols are as defined in the description of compound of formula (I) in pages 1-2.
by using reagents in such a way to obtain single isomeric form in the process wherever applicable or by conducting the reaction in the presence of reagents or catalysts in their single enantiomeric form. The single enantiomer, wherever applicable, may be prepared by resolving the racemic mixture by conventional methods. Some of the preferred methods include use of microbial resolution, resolving the diastereomeric salts formed with chiral bases such as S(+)-α-methylbenzylamine, R(−)-α-methylbenzylamine, S(+)-lysine, R(−)-lysine, S(+)—N-methyl-D-glucamine, R(−)—N-methyl-D-glucamine, R(−)-phenyl glycinol, S(+)-phenyl glycinol, S(+)-brucine, R(−)-brucine, cinchona alkaloids and their derivatives and the like wherever applicable Commonly used methods are compiled by Jaques et al in “Enantiomers, Racemates and Resolution” (Wiley Interscience, 1981) to obtain substantially pure stereoisomers of compounds of formula (I). Substantially pure means the material that contains at least 95%, preferably 98%, more preferably 99% of the compounds of formula (I).

It is appreciated that in any of the above-mentioned reactions, any reactive group in the substrate molecule may be protected according to conventional chemical practice. Suitable protecting groups in any of the above mentioned reactions are tertiarybutyldimethylsilyl, methoxymethyl, triphenyl methyl, benzyloxycarbonyl, tetrahydropyran(THP) etc, to protect hydroxyl or phenolic hydroxy group; N-tert-butoxycarbonyl (N-Boc), N-benzyloxycarbonyl (N-Cbz), N-9-fluorenyl methoxy carbonyl (—N-FMOC), benzophenoneimine, propargyloxy carbonyl (POC) etc, for protection of amino or anilino group, acetal protection for aldehyde, ketal protection for ketone and the like. The methods of formation and removal of such protecting groups are those conventional methods appropriate to the molecule being protected.

The compounds of the present invention can be used for the treatment of certain renal diseases including glomerulonephritis, glomerulosclerosis, nephrotic syndrome, hypertensive nephrosclerosis, nephropathy. The compounds of general formula (I) are also useful for the treatment/prophylaxis of insulin resistance (type II diabetes), leptin resistance, impaired glucose tolerance, dyslipidemia, disorders related to syndrome X such as hypertension, obesity, insulin resistance, coronary heart disease, and other cardiovascular disorders.

The compounds of the present invention may also be useful as aldose reductase inhibitors, for improving cognitive functions in dementia, as inflammatory agents, treating diabetic complications, disorders related to endothelial cell activation, psoriasis, polycystic ovarian syndrome (PCOS), inflammatory bowel diseases, osteoporosis, myotonic dystrophy, pancreatitis, retinopathy, arteriosclerosis, xanthoma and for the treatment of cancer.

The compounds of the present invention are useful in the treatment and/or prophylaxis of the above said diseases in combination/concomittant with one or more HMG CoA reductase inhibitors; cholesterol absorption inhibitors; antiobesity drugs; lipoprotein disorder treatment drugs; hypoglycemic agents: insulin; biguanides; sulfonylureas; thiazolidinediones; dual PPARα and γ or a mixture thereof. The compounds of the present invention in combination with HMG CoA reductase inhibitors, cholesterol absorption inhibitors, antiobesity drugs, hypoglycemic agents can be administered together or within such a period to act synergistically.

The invention is explained in detail in the examples given below which are provided by way of illustration only and therefore should not be construed to limit the scope of the invention.

### EXAMPLE 1

### Ethyl 2-[4-(3-biphenyl-4-yl-but-2-enyloxy)-phenoxy]-2-methyl-propanoate

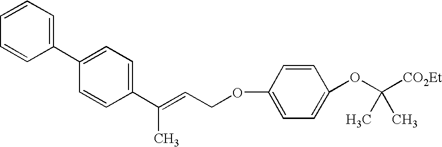

### Step (i): Preparation of 3-biphenyl-4-yl-but-2-enoic acid ethyl ester

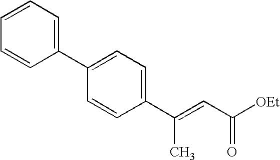

To the 60% NaH (3.06 grams, 0.127 mol) suspended in THF (50 mL) was added triethyl phoshonoacetate (12.69 mL, 0.637 mol) drop wise at 0° C. in dry THF (50 mL) with stirring under nitrogen atmosphere and the resulting solution was stirred at room temperature for 30 min and 4-acetyl biphenyl (10 grams, 0.051 mol) in THF (50 mL) was added drop wise at room temperature and the mixture was stirred at room temperature for 18 h, neutralized with 2 N HCl and extracted in to EtOAc. The combined organic layers were washed with water, dried over sodium sulphate and evaporated. The crude 3-biphenyl-4-yl-but-2-enoic acid ethyl ester was purified over silica gel column by eluting with 5% EtOAc:Pet.ether to give a trans product as white solid (8 grams, 59%). Mp. 77-79° C.

¹H NMR (δ, CDCl₃, 200 MHz): 7.70-7.30 (m, 9H), 6.21 (s, 1H), 4.23 (q, J=7.25 Hz, 2H), 2.62 (s, 3H), 1.33 (t, J=7.25 Hz, 3H).

### Step (ii): Preparation of 3-biphenyl-4-yl-but-2-ene-1-ol

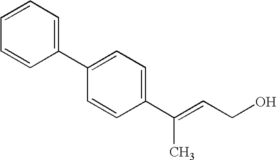

The 3-biphenyl-4-yl-but-2-enoic acid ethyl ester (8 grams), obtained in step (i), was reduced with AlH₃ (prepared from 4.22 grams of AlCl₃ and 3.61 g of LiAlH₄) in 200 mL of dry THF at −5° C. for 30 minutes. The reaction mixture was quenched with saturated Na₂SO₄ solution and filtered, washed with EtOAc and combined filtrates were evaporated to give 3-biphenyl-4-yl-but-2-ene-1-ol as a white low melting solid (Yield: 95%). Mp. 117-119° C.

¹H NMR (δ, CDCl₃, 200 MHz): 7.65-7.25 (m, 9H), 6.05 (t, J=6.72 Hz, 1H), 4.40 (d, J=6.72 Hz, 2H), 2.12 (s, 3H).

### Step (iii): Preparation of 2-[4-(3-Biphenyl-4-yl-but-2-enyloxy)-phenoxy]-2-methyl-propanoate

The 3-biphenyl-4-yl-but-2-ene-1-ol (0.455 g), obtained in step (ii), was coupled with the ethyl-4-hydroxy phenoxy-2-methyl-propanoate (Ref: J. Med. Chem. 2001, 44, 2061) (0.350 g) by Mitsinobu reaction using diisopropylazodicarboxylate (DIAD) (0.41 g) and PPh₃ (0.532 g) in TIE (10 mL) at 25° C. for 48 hours. The reaction was worked up by diluting with more of EtOAc and washing with aq.KHSO₄ solution and then with water. The dried solvent was evaporated and purified by column chromatography by eluting with 10% EtOAc and pet ether, to give 52% of the ethyl-2-[4-(3-Biphenyl-4-yl-but-2-enyloxy)-phenoxy]-2-methyl-propanoate as an thick oil.

¹H NMR (δ, CDCl₃, 200 MHz): 7.60-7.25 (m, 9H), 6.83 (s, 4H), 6.10 (t, J=6.35 Hz, 1H), 4.70 (d, 3=6.35 Hz, 2H), 4.23 (q, J=6.84 Hz, 2H), 2.15 (s, 3H), 1.53 (s, 6H), 1.27 (t, 3=6.84 Hz, 3H).

### EXAMPLE 2

### Ethyl 2-[4-(3-(4′-fluoro-biphenyl-4-yl-but-2-enyloxy)-phenoxy]-2-methyl-propanoate

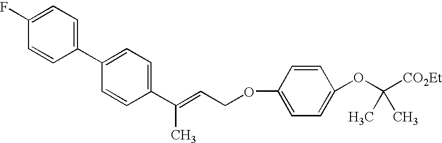

### Step (i): Preparation of 4-acetyl-4′-fluoro biphenyl

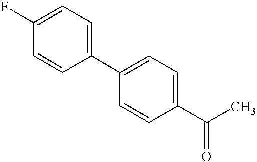

To a mixture of 4-fluoro bromobenzene (1 grams, 5.71 mmol) in 40 mL of dimethoxy ethane and Tetrakis palladium(0)(Pd(PPh₃)₄), (56 mg, 0.03 mmol) was added aqueous Na₂CO₃ solution (3.6 grams in 16 mL of water) and stirred at room temperature for 15 minutes and then was added 4-acetyl boronic acid (1.4 g, 8.56 mmol) and refluxed for 18 hours. The reaction mixture was acidified with 1 N HCl and extracted with EtOAc. The organic layer was washed wilt water and then with brine, dried over Na₂SO₄, evaporated and purified the crude product over silica gel column by eluting with 15% EtOAc+ Pet. ether to give 4-acetyl-4′-fluoro biphenyl as a creamish solid (0.98 grams, 75%).

¹H NMR (δ, CDCl₃, 200 MHz): 8.20-7.40 (m, 4H), 7.13 (d, J=8.60 Hz, 2H), 6.89 (d, J=8.60 Hz, 2H), 2.64 (s, 3H).

### Step (ii): Preparation of 3-(4′-fluoro-biphenyl-4-yl)but-2-enoic acid ethyl ester

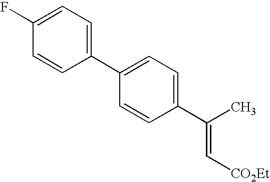

To the NaH (0.476 g, 5.91 mmol) in dry THF (5 mL) was added triethylphosphonoacetate (1.2 mL, 5.91 m mol) in 10 mL at 0° C. and stirred at room temperature for 30 min and then was added 4-acetyl-4′-fluoro biphenyl (0.9 grams, 3.94 mmol), obtained in step (i), in 10 mL of THF at room temperature and the mixture was stirred at room temperature for 16 h and quenched with ice-water neutralized with 1N HCl and extracted with EtOAc and washed with water, dried and evaporated to give a crude compound which was purified over silica gel column to give a creamish solid of 3-(4′-fluoro-biphenyl-4-yl)but-2-enoic acid ethyl ester as a E-isomer (0.44 grams, 48%).

¹H NMR (δ, CDCl₃, 200 MHz): 7.70-7.50 (m, 4H), 7.26 (d, J=8.59 Hz, 2H), 7.12 (d, J=8.59 Hz, 2H), 6.20 (s, 1H), 4.23 (q, J=6.99 Hz, 2H), 2.61 (s, 3H), 1.33 (t, J=6.99 Hz, 3H).

### Step (iii): Preparation of 3-(4′-fluoro-biphenyl-4-41)but-2-ene-1-ol

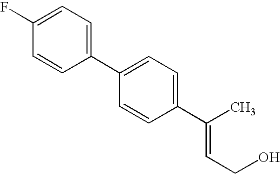

The 3-(4′-fluoro-biphenyl-4-yl)but-2-enoic acid ethyl ester (0.44 g, 1.54 mmol), obtained in step (ii), was reduced with AlH₃ (prepared from LAH (0.176 grams) and AlCl₃ (0.206 grams) in dry THF (10 mL) at −5° C. for 30 minutes. The reaction mixture was quenched with sat.Na₂SO₄ solution and filtered, washed with EtOAc and combined filtrates were evaporated to give 3-(4′-fluoro-biphenyl-4-yl)but-2-ene-1-ol as a white low melting solid (Yield: 0.35 g, 95%).

¹H NMR (δ, CDCl₃, 200 MHz): 7.65 (m, 4H), 7.23 (d, J=8.59 Hz, 2H), 7.11 (d, J=8.59 Hz, 2H), 6.06 (t, 3=6.98 Hz, 1H), 4.41 (d, J=6.98 Hz, 2H), 2.13 (s, 3H).

### Step (iv): Preparation of ethyl-2-[4-[3-(4′-fluoro-biphenyl-4-yl)-but-2-enyloxy]phenoxy]-2-methylpropanoate

The 3-(4′-fluoro-biphenyl-4-yl)but-2-ene-1-ol (0.350 grams), obtained in step (iii), was coupled with the ethyl-4-hydroxy phenoxy-2-methyl-propanoate (Ref: JMC, 2001, 44, 2061) (0.323 grams) by Mitsinobu reaction using DIAD (0.436 grams) and PPh₃ (0.572 grams) in THF (10 mL) at 25° C. for 48 hours. The reaction was worked up by diluting with more of EtOAc and washing with aqueous KHSO₄ solution and then with water, the dried solvent was evaporated and purified by column chromatography by eluting with 10% EtOAc and Pet. ether, to give 27% (0.17 grams) of the ethyl-2-[4-[3-(4′-fluoro-biphenyl-4-yl)-but-2-enyl]phenoxy]-2-methyl-propanoate as an thick oil.

¹H NMR (δ, CDCl₃, 200 MHz): 7.60-7.50 (m, 8H), 7.14 (d, J=8.59 Hz, 2H), 6.84 (d, J=8.59 Hz, 2H), 6.10 (t, =6.45 Hz, 1H), 4.71 (d, J=6.45 Hz, 2H), 4.24 (q, J=7.25 Hz, 2H), 2.15 (s, 3H), 1.56 (s, 6H), 1.28 (t, J=7.25 Hz, 3H).

The following compounds falling into the general formula (I) have also been prepared by the process as defined in examples 1 and 2.

| Example | | |
|---|---|---|
| No. | Structure | Analytical Data |
| 3 | 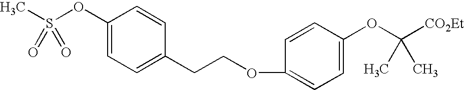 | ¹H NMR (δ, CDCl₃, 200 MHz): 7.34-7.23(m, 4H), 6.85-6.72(m, 4H), 4.22(q, J=7.03Hz, 2H), 4.10(t, J==6.70Hz, 2H), 3.12(s, 3H), 3.07(t, J==6.70Hz, 2H), 1.52(s, 6H), 1.27(t, J=7.03Hz, 3H). Nature: Liquid. |
| 4 | 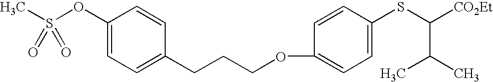 | ¹H NMR (δ, CD₃OD, 200 MHz): 7.40(d, J=8.59Hz, 2H), 7.30-7.15(m, 4H), 6.81(d, J=8.59Hz, 2H), 4.10(q, J=6.99Hz, 2H), 3.94(t, J=5.91Hz, 2H), 3.25(d, J=9.13Hz, 1H), 3.13(s, 3H), 2.82(t, J=8.06Hz, 2H), 2.10-1.95(m, 3H), 1.25(t, J=6.99Hz, 3H), 1.17(d, 6.35Hz, 3H), 1.03(d, J=6.35Hz, 3H). Nature: Liquid. |
| 5 | 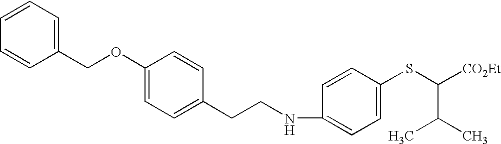 | ¹H NMR (δ, CDCl₃, 200 MHz): 7.42-7.20(m, 7H), 7.11(d, J=8.30Hz, 2H), 6.92(d, J=8.30Hz, 2H), 6.65(d, J=8.30Hz, 2H), 5.05(s, 2H), 4.09(q, J=7.33Hz, 2H), 3.35(t, J=6.84Hz, 2H), 3.21(d, J=9.28Hz, 1H), 2.88(t, J=6.84Hz, 2H), 2.20-2.00(m, 1H), 1.22(t, J=7.33Hz, 3H), 1.14(d, |
| #J=6.83Hz, 3H) 1.02(d, J=6.83Hz, Nature: Liquid | | |
| 6 | 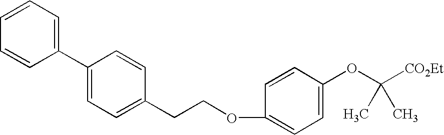 | ¹H NMR (δ, CDCl₃, 200 MHz): 7.60-7.12(m, 9H), 6.80-6.86(m, 4H), 4.12-4.29(m, 4H), 3.11(t, J=6.80Hz, 2H), 1.53(s, 6H), 1.27(t, J=7.0Hz, 3H). Nature: liquid |
| 7 | 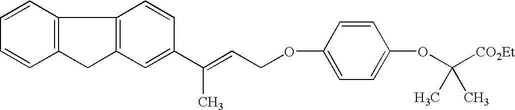 | ¹H NMR (δ, CDCl₃, 200 MHz): 7.80-7.20(m, 7H), 6.94(d, J=9.67Hz, 2H), 6.88(d, J=9.67Hz, 2H), 6.11(t, J=6.18Hz, 1H), 4.74(d, J=6.18Hz, 2H), 3.90(s, 2H), 2.20(s, 3H), 1.54(s, 6H). M.P: 155-158° C. |
| 8 | 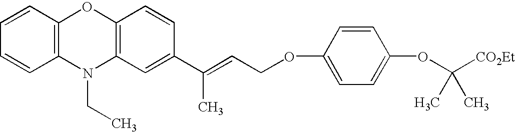 | ¹H NMR (δ, CDCl₃, 200 MHz): 6.92-6.60(m, 11H), 5.96(m, 1H), 4.67(d, J=6.10Hz, 2H), 4.15(q, J=7.10Hz, 2H), 3.71-3.68(m, 2H), 2.03(s, 3H), 1.45(s, 6H), 1.28-1.10(m, 6H). Nature: Liquid |
| 9 | 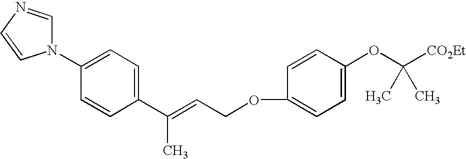 | ¹H NMR (δ, CDCl₃, 200 MHz): 8.40(s, 1H), 7.61(d, J=6.83Hz, 2H), 7.60-7.39(m, 4H), 6.88(d, J=9.27Hz, 2H), 6.85(d, J=9.27Hz, 2H), 6.13(t, J=4.88Hz, 1H), 4.73(d, J=4.88Hz, 2H), 4.27(q, J=7.31Hz, 2H), 2.19(s, 3H), 1.58(s, 6H), 1.32(t, J=7.31Hz, 3H). |
| 10 | 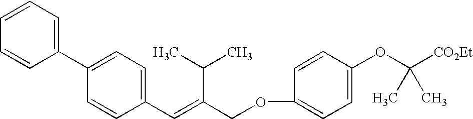 | ¹H NMR (δ, CDCl₃, 200 MHz): 7.60-7.20(m, 9H), 6.83(d, J=9.14Hz, 2H), 6.78(d, J=9.14Hz, 2H), 6.64(s, 1H), 4.57(s, 2H), 4.23(q, J=7.25Hz, 2H), 2.80-2.60(m, 1H), 1.56(s, 6H), 1.54(d, J=6.34Hz, 3H), 1.22(t, J=7.25Hz, 3H), 1.19(d, J=6.34Hz, 3H). Nature: Viscous liquid. |
| 11 | 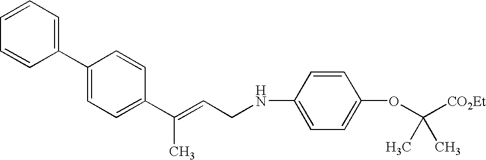 | ¹H NMR (δ, CDCl₃, 200 MHz): 7.61-7.30(m, 9H), 6.80(d, J=8.50Hz, 2H), 6.55(d, J=8.50Hz, 2H), 6.00-5.97(m, 1H), 4.23(q, J=7.00Hz, 2H), 3.90(d, J=6.40Hz, 2H), 2.15(s, 3H), 1.52(s, 6H), 1.28(t, J=7.00Hz, 3H). Nature: liquid. |
| 12 | 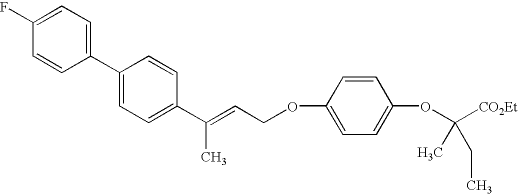 | ¹H NMR (δ, CDCl₃, 200 MHz): 7.44-7.20(M, 8H), 6.93(d, J=2.44Hz, 2H), 6.92(d, J=2.44 Hz, 2H), 6.04(t, J=6.45Hz, 1H), 4.69(d, J=6.45Hz, 2H), 4.25(q, J=6.98Hz, 2H), 2.14 (s, 3H), 1.95(q, J=7.52Hz, 2H), 1.43(s, 3H), 0.99(t, J=7.52Hz, 3H), 0.88(t, J=6.98Hz, 3H). Nature: liquid. |
| 13 | 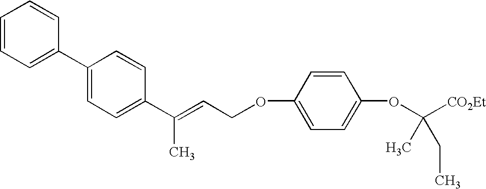 | ¹H NMR (δ, CDCl₃, 200 MHz): 7.63-7.30(m, 9H); 6.85(d, J=9.40Hz, 2H); 6.78(d, J=9.40Hz, 2H); 6.11(t, J=6.50Hz, 1H); 4.71(d, J=6.50Hz, 2H); 4.25(q, J=7.10Hz, 2H); 2.16(s, 3H); 1.95(q, J=7.52Hz, 2H); 1.44 (s, 3H); 1.29(t, J=7.10Hz, 3H); 0.99(t, J=7.52Hz, 3H). Nature: gummy liquid |
| 14 | 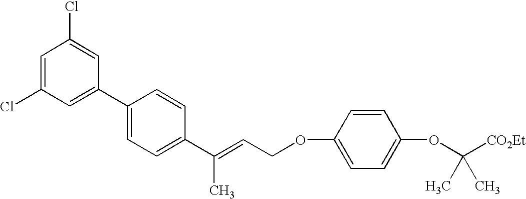 | ¹H NMR (δ, CDCl₃, 200 MHz): 7.60-7.25(m, 7H); 6.86(s, 4H); 6.13(t, J=5.86Hz, 1H); 4.72(d, J=5.86Hz, 2H); 4.25(q, J=7.32Hz, 2H); 2.16(s, 3H); 1.56(s, 6H); 1.29(t, J=7.32Hz, 3H). Nature: Liquid. |
| 15 | 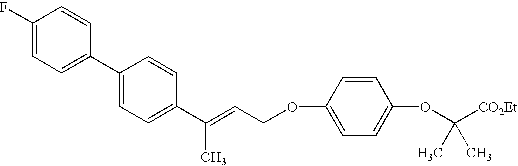 | ¹H NMR (δ, CDCl₃, 200 MHz): 7.60-7.00(m, 8H); 6.91(d, J=5.86Hz, 2H); 6.89(d, J=5.86Hz); 6.09(t, J=6.18Hz, 1H); 4.73(d, J=6.18Hz, 2H); 2.17(s, 3H); 1.54(s, 6H). |
| 16 | 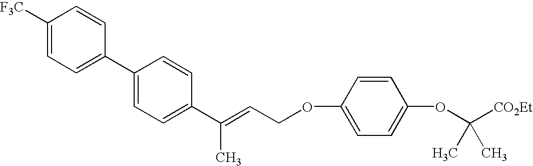 | ¹H NMR (δ, CDCl₃, 200 MHz): 7.60-7.20(m, 8H); 6.90(d, J=6.85Hz, 2H); 6.88(d, J=6.85Hz, 2H); 6.11(t, J=6.11Hz, 1H); 4.76 (d, J=6.11Hz, 2H); 4.12(q, J=7.01Hz, 2H), 2.18(s, 3H); 1.46(s, 6H), 1.23(t, J=7.01Hz, 3H). Nature: Liquid. |
| 17 | 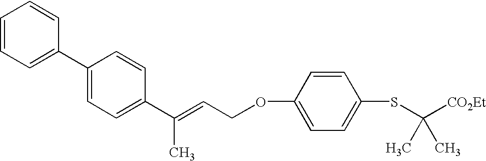 | ¹H NMR (δ, CDCl₃, 200 MHz): 7.63-7.33(m, 11H); 6.91(d, J=8.30Hz, 2H); 6.12 (t, J=8.40Hz, 1H); 4.77(d, J=6.40 Hz, 2H); 4.12(q, J=7.20Hz, 2H); 2.19(s, 3H); 1.40(s, 6H); 1.23(t, J=7.20Hz, 3H). Nature: liquid. |
| 18 | 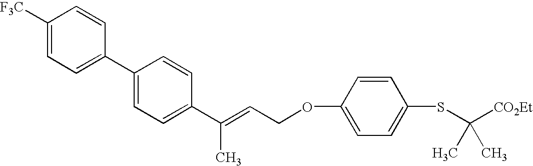 | ¹H NMR (δ, CDCl₃, 200 MHz): 7.78-7.50(m, 8H); 7.40(d, J=8.50Hz, 2H); 6.92(d, J=8.50Hz, 2H); 6.11(t, J=6.15Hz, 1H); 4.76(d, J=6.15Hz, 2H); 4.12(q, J=7.0Hz, 2H); 2.18(s, 3H); 1.46(s, 6H); 1.23(t, J=7.0Hz, 3H). |
| 19 | 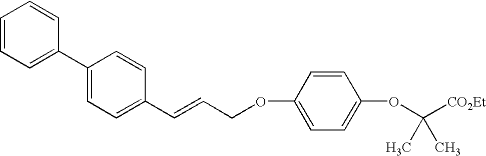 | ¹H NMR (δ, CDCl₃, 200 MHz): 7.70-7.20(m, 9H); 6.84 (s, 4H); 6.75(t, J=8.08Hz, 1H); 6.50-6.40(m, 1H); 4.66(d, J=5.91Hz, 2H); 4.24(q, J=7.25Hz, 2H); 1.54(s, 6H); 1.26(t, J=7.52Hz, 3H). Nature: Liquid. |
| 20 | 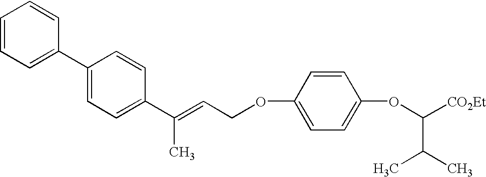 | ¹H NMR (δ, CDCl₃, 200 MHz): 7.64-7.32(m, 9H); 6.88(s, 4H); 6.13(t, J=5.86Hz, 1H); 4.72(d, J=5.86Hz, 2H); 4.24(q, J=7.33Hz, 2H); 2.40-2.20(m, 1H), 2.18(s, 3H); 1.27(t, J=7.33Hz, 3H); 1.10(d, J=4.88 Hz, 6H) |
| 21 | 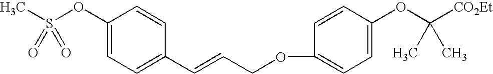 | ¹H NMR (δ, CDCl₃, 200 MHz): 7.445(d, J=8.33Hz, 2H); 7.23(d, J=8.33Hz, 2H); 6.83(d, J=9.28 Hz, 2H); 6.78(d, J=9.28Hz, 2H), 6.69(d, J=16.12Hz, 1H); 6.45-6.31(m, 1H); 4.65(d, J=5.37Hz, 2H); 4.22(q, J=7.25Hz, 2H), 3.14 (s, 3H); 1.54(s, 6H); 1.31-1.28(t, J=7.25Hz, 3H). Nature: Gummy solid. |
| 22 | 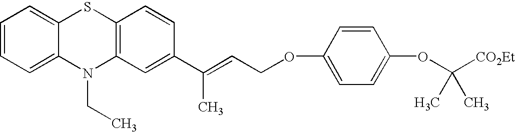 | ¹H NMR (δ, DMSO-d⁶, 200 MHz): 7.20-6.76(m, 11H); 6.01(t, J=7.33Hz, 1H); 4.67(d, J=5.60Hz, 2H); 4.15(q, J=7.0Hz, 2H), 3.96(q, J=6.72Hz, 2H); 2.07(s, 3H); 1.45(s, 6H); 1.33-1.15(m, 3H); 0.99(t, J=70Hz, 3H). |
| 23 | 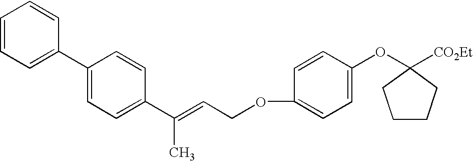 | ¹H NMR (δ, CDCl₃, 200 MHz): 7.63-7.31(m, 9H), 6.89(d, J=9.28Hz, 2H), 6.78(d, J=9.28Hz, 2H), 6.11(t, J=5.86Hz, 1H), 4.71(d, J=5.86Hz, 2H), 4.22(q, J=7.33Hz, 2H), 2.40-2.00(m, 7H), 1.90-1.70(m, 4H), 1.21(t, J=7.33Hz, 3H). M.P. 112-114° C. |
| 24 | 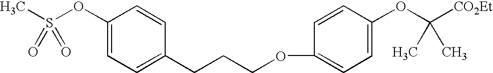 | ¹H NMR (δ, CDCl₃, 200 MHz): 7.26(d, J=6.17Hz, 2H), 7.20(d, J=6.17Hz, 2H), 6.83(d, J=6.72Hz, 2H), 6.75(d, J=6.72Hz, 2H), 4.24(q, J=6.98Hz, 2H), 3.90(t, J=5.91Hz, 2H) 3.13(s, 3H), 2.81(t, J=7.25Hz, 2H), 2.10-2.02(m, 2H), 1.53(s, 6H), 1.28(t, J=6.98Hz, 3H). |

Some more examples of compound of formula (I) which can be prepared by the person skilled in the art by following the procedure as described in example 1:

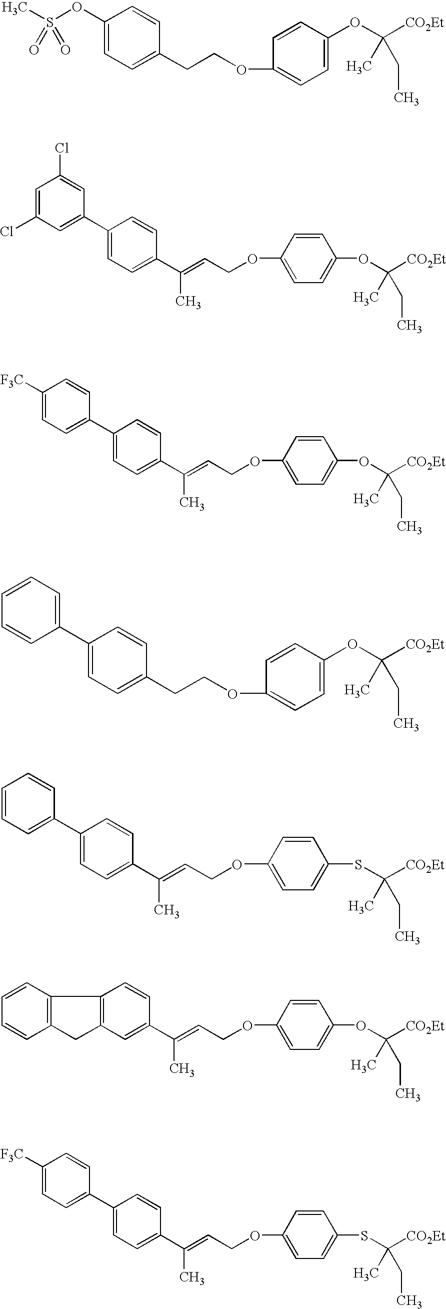

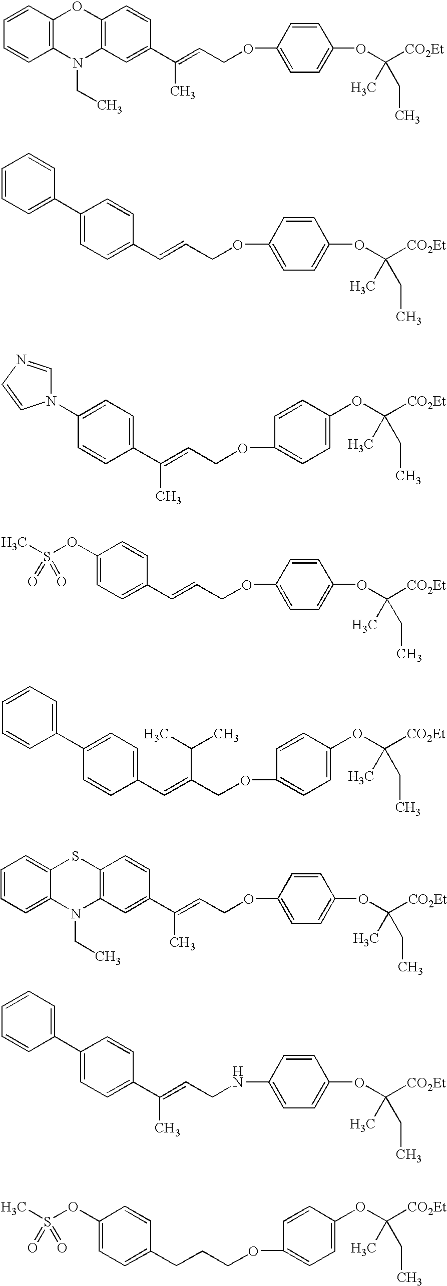

### EXAMPLE 25

### 2-[4-(3-Biphenyl-4-yl-but-2-enyloxy)phenoxy]-2-methyl propanoic acid

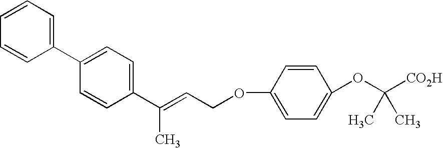

Ethyl-2-[4-(3-biphenyl-4-yl-but-2-enyloxy)-phenoxy]-2-methyl-propanoate (0.35 grams), obtained in example 1, was hydrolysed with aqueous LiOH (0.35 grams in 2 mL of water) at 25° C. for 12 hours in methanol. THF mixture (3 mL+2 mL) after the completion of reaction the solvent was evaporated and the aqueous layer was washed once with ether and the aqueous layer was acidified with 2 N HCl to pH 2 and extracted with EtOAc and the organic layer was dried with Na₂SO₄ and evaporated under reduced pressure to give the title compound as a white solid in 90% yield. Mp. 148-150° C.

¹H NMR (δ, CDCl₃, 200 MHz): 7.60-7.25 (m, 9H), 6.88 (s, 4H), 6.08 (t, J=6.35 Hz, 1H), 4.72 (d, J=6.35 Hz, 2H), 2.15 (s, 3H), 1.49 (s, 6H).

### EXAMPLE 26

### 2-[4-3-(4′-Fluoro-biphenyl-4-yl-but-2-enyloxy)-phenoxy]-2-methyl-propanoic acid

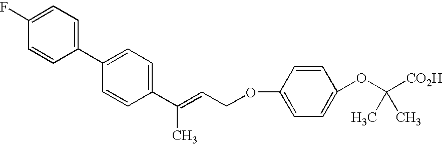

Ethyl-2-[4-[3-(4′-fluoro-biphenyl-4-yl)-but-2-enyloxy]phenoxy]-2-methylpropanoate (0.17 grams), obtained in example 2, was hydrolysed with aqueous LiOH (0.79 grams in 1 mL of water) at 25° C. for 12 hours in methanol:THF mixture (3 mL+2 mL). After completion of the reaction the solvent was evaporated and the aqeous layer was washed once with ether and the aqueous layer was acidified with 2 N HCl to pH 2 and extracted with EtOAc and the organic layer was dried with Na₂SO₄ and evaporated under reduced pressure to give the title compound as a white solid (Yield: 59%, 0.10 grams). Mp. 148-150° C.

¹H NMR (200 MHz): δ 7.60-7.00 (m, 8H), 6.91 (d, J=5.86 Hz, 2H), 6.89 (d, J=5.86 Hz, 2H), 6.09 (t, J=6.18 Hz, 1H), 4.73 (d, J=6.18 Hz, 2H), 2.17 (s, 3H), 1.54 (s, 6H).

The following compounds falling into the general formula (I) have also been prepared by the process as defined in examples 25 and 26.

| Example | | |
|---|---|---|
| No. | Structure | Analytical Data |
| 27 | 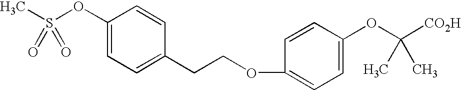 | ¹H NMR (δ, CDCl₃, 200 MHz): 7.35-7.25(m, 4H), 6.90(d, J=9.10Hz, 2H), 6.79(d, J=9.10Hz, 2H), 4.13(t, J=6.7Hz, 2H), 3.14(s, 3H), 3.07(t, J=6.7Hz, 2H), 1.53(s, 6H). Nature: Liquid. |
| 28 | 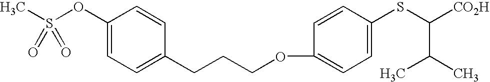 | ¹H NMR (δ, CD₃OD, 200 MHz): 7.41(d, J=8.79Hz, 2H), 7.02(d, J=8.30Hz, 2H), 6.86(d, J=8.79Hz, 2H), 6.69(d, J=8.30Hz, 2H), 3.94(t, J=6.34Hz, 2H), 3.32(s, 3H), 3.22(d, J=9.28Hz, 1H), 2.70(t, J=8.31Hz, 2H), 2.10-1.95(m, 3H), 1.16(d, 6.35Hz, 3H), 1.04(d, J=6.35Hz, 3H). M.P: 115-118° C. |
| 29 | 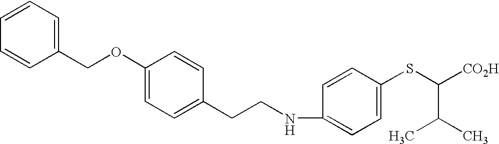 | ¹H NMR (δ, CDCl₃, 200 MHz): 7.43-7.10(m, 9H), 6.90(d, J=8.60Hz, 2H), 6.52(d, J=8.60Hz, 2H), 5.04(s, 2H), 3.25(t, J=7.5Hz, 2H), 3.11(d, J=8.8Hz, 1H), 2.78(t, J=7.5Hz, 2H), 2.0-1.90(m, 1H), 1.08(d, J=6.7Hz, 3H), 1.02(d, J=6.7Hz, 3H). Nature: Liquid |
| 30 | 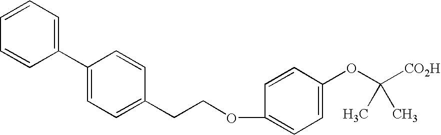 | ¹H NMR (δ, CDCl₃, 200 MHz): 7.66-7.58(m, 4H), 7.48-7.34(m, 5H), 6.83-6.77(m, 4H), 4.15(t, J=6.6Hz, 2H), 3.02(t, J=6.6Hz, 2H), 1.42(s, 6H). M.P: 138-140° C. |
| 31 | 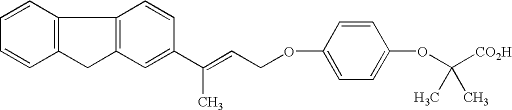 | ¹H NMR (δ, CDCl₃, 200 MHz): 7.80-7.20(m, 7H), 6.94(d, J=9.67Hz, 2H), 6.88(d, J=9.67Hz, 2H), 6.11(t, J=6.18Hz, 1H), 4.74(d, J=6.18Hz, 2H), 3.90(s, 2H), 2.20(s, 3H), 1.54(s, 6H). M.P: 155-158° C. |
| 32 | 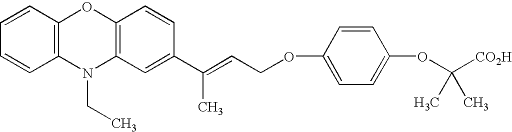 | ¹H NMR (δ, CDCl₃, 200 MHz): 6.81(m, 11H), 5.95(m, 1H), 4.62(d, J=5.8Hz, 2H), 3.69(q, J=6.30Hz, 2H), 2.02(s, 3H), 1.35(s, 6H), 1.12(t, J=6.30Hz, 3H). M.P: 114-116° C. |
| 33 | 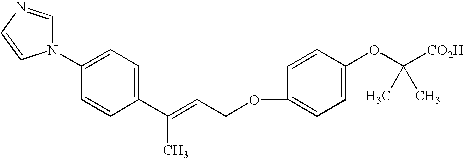 | ¹H NMR (δ, DMSO-d⁶, 200 MHz): 8.24(s, 1H), 7.73(bs, 1H), 7.60(d, J=6.83Hz, 2H), 7.57(d, J=6.83Hz, 2H), 7.09(bs, 1H), 6.88(d, J=8.77Hz, 2H), 6.08(t, J=5.85Hz, 1H), 4.70(d, 5.85Hz, 2H), 2.11(s, 3H), 1.43(s, 6H). |
| 34 | 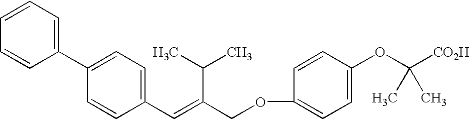 | ¹H NMR (δ, CDCl₃, 200 MHz): 7.60-7.20(m, 9H), 6.65(s, 1H), 4.60(s, 2H), 2.80-2.60(m, 1H), 1.53(s, 6H), 1.24(d, J=6.34Hz, 3H), 1.19(d, J=6.34Hz, 3H). Nature: Viscous liquid. |
| 35 | 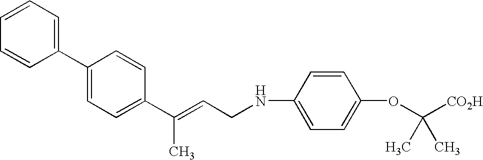 | ¹H NMR (δ, CDCl₃, 200 MHz): 7.65-7.53(m, 3H), 7.49-7.35(m, 6H), 6.71(d, J=8.60Hz, 2H), 6.50(d, J=8.60Hz, 2H), 5.96(m, 1H), 3.81(d, J=5.90Hz, 2H), 2.11(s, 3H), 1.37(s, 6H) M.P: 184-188° C. |
| 36 | 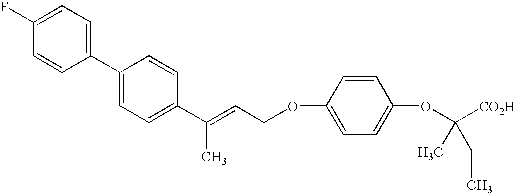 | ¹H NMR (δ, CDCl₃, 200 MHz): 7.44-7.20(M, 2H), 6.93(d, J=2.44Hz, 2H), 6.92(d, J=2.44 Hz, 2H), 6.02(t, J=6.35Hz, 1H), 4.71(d, J=6.35Hz, 2H), 2.14(s, 3H), 2.0-1.80(m, 2H), 1.42(s, 3H), 1.06(t, J=7.32Hz, 3H). M.p: 122-125° C. |
| 37 | 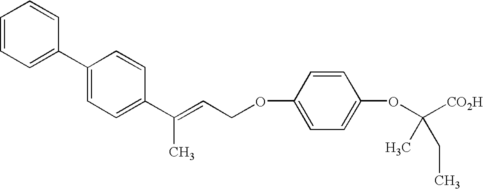 | ¹H NMR (200 MHz): δ 7.70-7.30 (m, 9H), 6.96(d, J=9.40Hz, 2H), 6.89(d, J=9.40Hz, 2H), 6.12(t, J=6.18Hz, 1H), 4.75(d, J=6.18Hz, 2H), 2.19(s, 3H), 1.97(q, J=7.52Hz, 2H), 1.45(s, 3H), 1.08(t, J=7.52Hz, 3H). Mp: 114-117° C. |
| 38 | 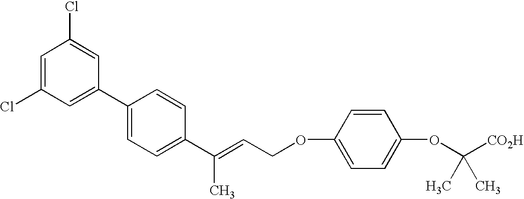 | ¹H NMR (200 MHz): δ 7.60-7.25 (m, 7H), 6.93(d, J=5.37Hz, 2H), 6.87(d, J=5.37Hz, 2H), 6.13(t, J=5.86Hz, 1H), 4.75(d, J=5.86Hz, 2H), 2.18(s, 3H), 1.57(s, 6H). Mp: 52-54° C. |
| 39 | 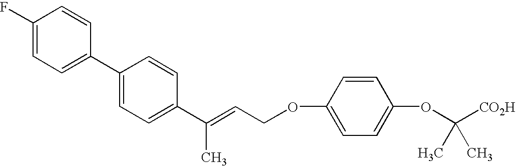 | ¹H NMR (δ, CDCl₃, 200 MHz): 7.60-7.40(m, 6H), 7.30-7.00(m, 2H), 6.91(d, J=9.20Hz, 2H), 6.89(d, J=9.20Hz, 2H), 6.09(t, J=6.18Hz, 1H), 4.73(d, J=6.18Hz, 2H), 2.17(s, 3H), 1.54(s, 6H). M.P. 148-150° C. |
| 40 | 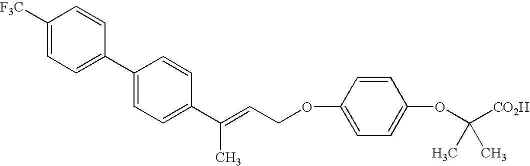 | ¹H NMR (200 MHz): δ 7.60-7.20 (m, 8H), 6.91(d, J=6.86Hz, 2H), 6.89(d, J=6.86Hz, 2H), 6.09(t, J=6.48Hz, 1H), 4.73(d, J=6.48Hz, 2H), 2.12(s, 3H), 1.49(s, 6H). Mp: 150-154° C. |
| 41 | 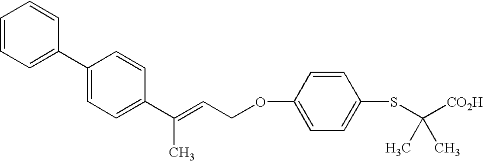 | ¹H NMR (200 MHz): δ 7.70-7.25 (m, 11H), 6.92(d, J=8.59Hz, 2H), 6.10(t, J=6.17Hz, 1H), 4.76(d, J=6.17Hz, 2H), 2.16(s, 3H), 1.49(s, 6H) Mp: 162-165° C. |
| 42 | 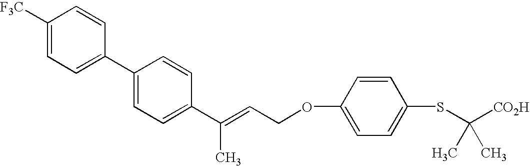 | ¹H NMR (200 MHz): δ 7.78-7.50 (m, 8H), 7.46(d, J=8.86Hz, 2H), 6.92(d, J=8.86Hz, 2H), 6.11(t, J=6.45Hz, 1H), 4.77(d, J=6.45Hz, 2H), 2.17(s, 3H), 1.49(s, 6H). Mp: 142-146° C. |
| 43 | 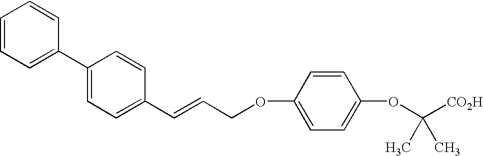 | ¹H NMR (200 MHz): δ 7.80-7.25 (m, 9H), 6.92(d, J=8.86Hz, 2H), 6.82(d, J=8.86Hz, 2H), 6.64(d, J=9.27Hz, 1H), 6.62-6.40(m, 1H), 4.68(d, J=5.37 Hz, 2H), 1.44(s, 6H) Mp: 172-174° C. |
| 44 | 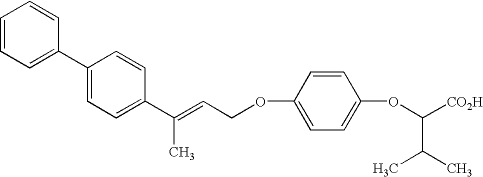 | ¹H NMR (200 MHz): δ 7.80-7.30 (m, 9H), 6.92(d, J=9.13Hz, 2H), 6.82(d, J=9.13Hz, 2H), 6.09(t, J=6.18Hz, 1H), 4.72(d, J=6.18Hz, 2H), 4.34(d, J=5.11 Hz, 1H), 2.30-2.00(m, 4H), 1.02(d, J=5.72Hz, 3H), 0.99(d, J=5.72Hz, 3H). Mp: 136-140° C. |
| 45 | 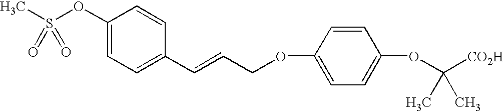 | ¹H NMR (200 MHz): δ 7.45(d, J=8.79Hz, 2H), 7.25(d, J=8.79 Hz, 2H), 6.92(d, J=9.28Hz, 2H), 6.84(d, J=9.28Hz, 2H), 6.71(d, J=16.11Hz, 1H), 6.50-6.30(m, 1H), 4.65(d, J=5.37 Hz, 2H), 3.15(s, 3H), 1.54(s, 6H) Mp: 112-114° C. |
| 46 | 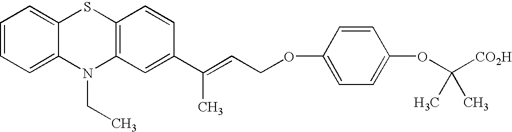 | ¹H NMR (200 MHz): δ 7.20-6.78 (m, 11H), 6.01(t, J=7.33Hz, 1H), 4.67(d, J=7.33Hz, 2H), 3.95(q, J=6.82Hz, 2H), 2.06(s, 3H), 1.43(s, 6H), 1.29(t, J=6.82 Hz, 3H). |
| 47 | 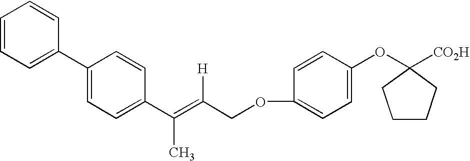 | ¹H NMR (200 MHz): δ 7.70-7.30 (m, 9H), 6.87(d, J=9.28Hz, 2H), 6.79(d, J+9.28Hz, 2H), 6.10(t, J=5.86Hz, 1H), 4.71(d, J=5.8Hz, 2H), 2.35-2.18(m, 4H), 2.16(s, 3H), 1.90-1.70(m, 4H) Mp: 142-144° C. |
| 48 | 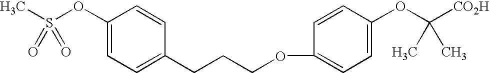 | ¹H NMR (δ, CDCl₃, 200 MHz): 7.34(d, J=8.30Hz, 2H), 7.26(d, J=8.30Hz, 2H), 6.82(s, 4H), 3.91(t, J=6.35Hz, 2H), 2.75(t, J=7.33Hz, 2H), 2.03-1.90(m, 2H), 1.43(s, 6H). M.P: 64-66° C. |
| 49 | 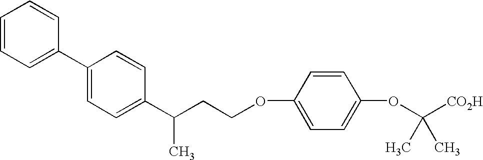 | ¹H NMR (200 MHz): δ 7.65-7.20(m, 9H), 6.85(d, J=8.79Hz, 2H), 6.74(d, J=8.79Hz, 2H), 3.90-3.75(m, 2H), 3.20-3.0(m, 1H), 2.20-2.00(m, 2H), 1.47(s, 6H), 3.33(d, J=6.86Hz, 3H). Mp: 120-122° C. |

Some more examples of compound of formula (I) which can be prepared by the person skilled in the art by following the procedure as described in examples 25 and 26:

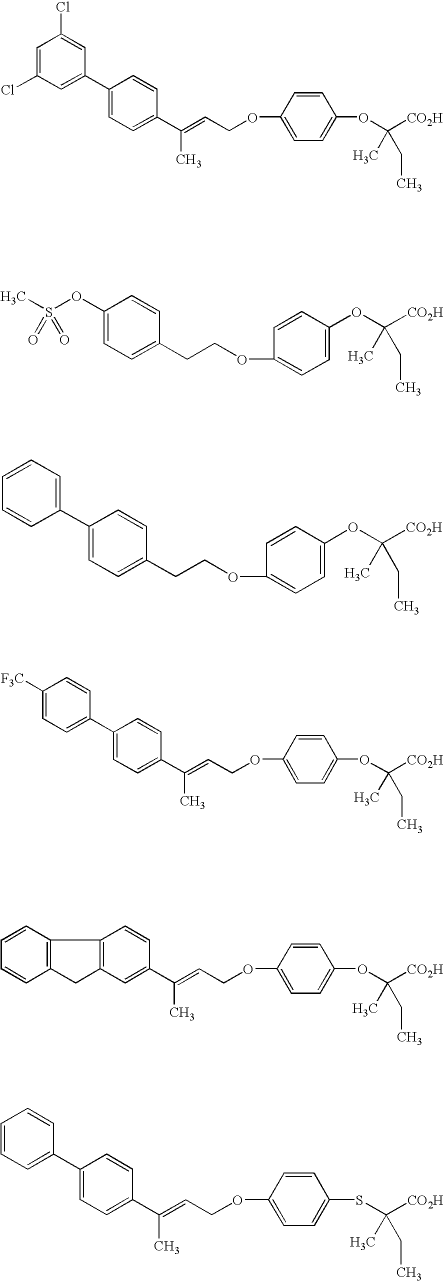

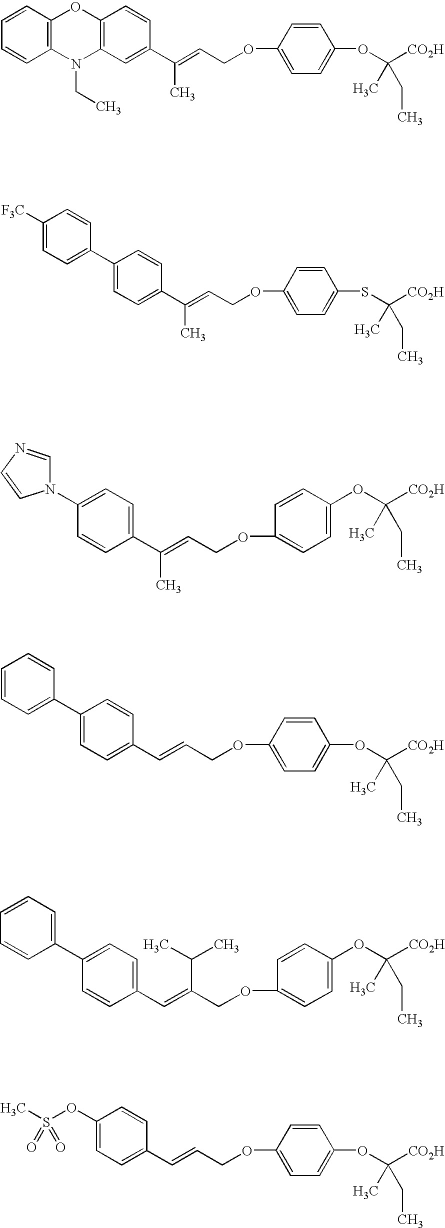

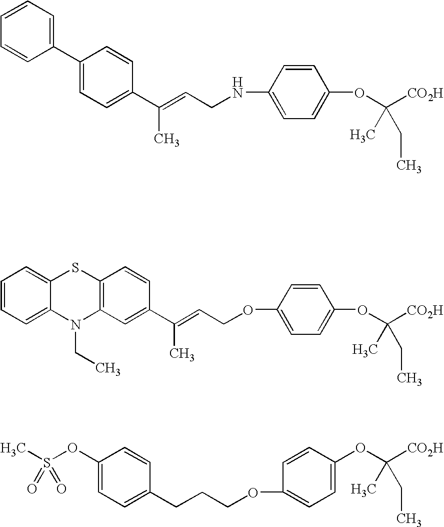

### EXAMPLE 50

### S(−)-2-[4-(3-Biphenyl-4-yl-but-2-enyloxy)phenoxy]2-methyl butyric acid & R(+)-2-[4-(3-Biphenyl-yl-but-2-enyloxy)phenoxy]2-methyl butyric acid

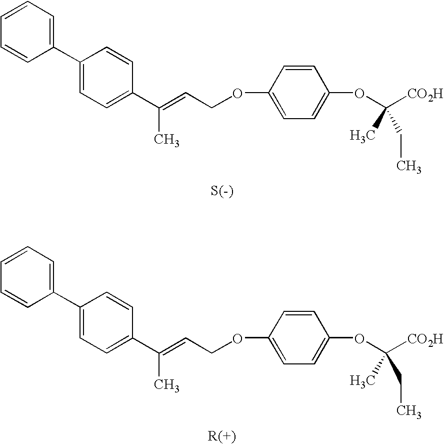

### Step (i): Preparation of ethyl 2-[4-(3-biphenyl-4-yl-but-2-enyloxy)-phenoxy]-2-methy butanoate

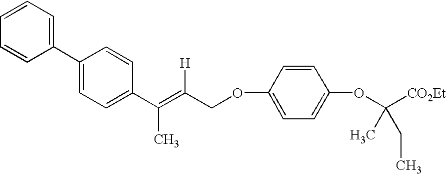

The 3-biphenyl-4-yl-but-2-ene-1-ol (0.455 grams), obtained in step (ii) of Example 1, was coupled with the ethyl-4-hydroxy phenoxy-2-methyl butanoate (Ref: J. Med. Chem. 2001, 44, 2061) (0.350 grams) by Mitsiniobu reaction using diisopropylazodicarboxylate (DIAD) (0.41 grams) and PPh₃ (0.532 grams) in THF (10 mL) at 25° C. for 48 hours. The reaction was worked up by diluting with more of EtOAc and washing with aq.KHSO₄ solution and then with water. The dried solvent was evaporated and purified by column chromatography by eluting with 10% EtOAc and pet ether, to give 52% of the ethyl-2-[4-(3-Biphenyl-4-yl-but-2-enyloxy)-phenoxy]-2-methyl-propanoate as an thick oil.

¹H NMR (δ, CDCl₃, 200 MHz): 7.63-7.30 (m, 9H); 6.85 (d, J=9.40 Hz, 2H); 6.78 (d, J=9.40 Hz, 2H); 6.11 (t, J=6.50 Hz, 1H); 4.71 (d, J=6.50 Hz, 2H); 4.25 (q, J=7.10 Hz, 2H), 2.16 (s, 3H); 1.95 (q, J=7.52 Hz, 2H); 1.44 (s, 3H); 1.29 (t, J=7.10 Hz, 3H); 0.99 (t, J=7.52 Hz, 3H)

### Step (ii): Preparation of 2-[4-(3-Biphenyl-4-yl-but-2-enyloxy)-phenoxy]-2-methy butanoic acid

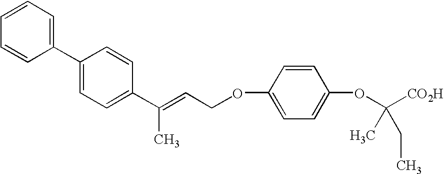

Ethyl 2-[4-(3-biphenyl-4-yl-but-2-enyloxy)-phenoxy]-2-methy butanoate (0.17 grams), obtained in step (i) above, was hydrolysed with aqueous LiOH (0.79 grams in 1 mL of water) at 25° C. for 12 hours in methanol:THF mixture (3 mL+2 mL). After completion of the reaction the solvent was evaporated and the aqeous layer was washed once with ether and the aqueous layer was acidified with 2 N HCl to pH 2 and extracted with EtOAc and the organic layer was dried with Na₂SO₄ and evaporated under reduced pressure to give the title compound as a white solid (Yield: 63%, 0.10 grams).

Mp: 114-117° C.

¹H NMR (200 MHz): δ 7.70-7.30 (m, 9H), 6.96 (d, J=9.40 Hz, 2H), 6.89 (d, J=9.40 Hz, 2H), 6.12 (t, J=6.18 Hz, 1H), 4.75 (d, J=6.18 Hz, 2H), 2.19 (s, 3H), 1.97 (q, 3=7.52 Hz, 2H), 1.45 (s, 3H), 1.08 (t, J=7.52 Hz, 3H).

### Step (iii): Resolution of 2-[4(3-Biphenyl-4-yl-but-2-enyloxy)-phenoxy]-2-methy butanoic acid

To the 2-[4-(3-biphenylyl-but-2-enyloxy)-phenoxy]-2-methyl-butanoic acid (±) (racemic mixture) (11.2 g, 26.92 mmol), obtained in step (ii) above, in methanol (100 mL; slightly warm it if necessary to dissolve the compound and then cool it) was added R (−) phenyl glycenol and stirred the mixture for 12 hours at room temperature and the methanol was evaporated at low pressure and dried under vacuum to give 14.7 grams of the salt as a creamish solid. M.P: 144-148° C. for R (−) phenyl glycenol salt; M.P: 150-155° C. Similarly in another experiment, to the racemic acid (11.2 grams, 26.92 mmol) was added S(+) phenyl glycenol (3.69 grams, 26.92 mmol) and did the reaction as explained above to get the S(+) phenyl glycenol salt; M.P: 178-180° C.

The above salt of either ‘R’ or ‘S’ phenylglycenol (14.7 g) was washed with 3:1 of tertiary butyl methyl ether(TBME): Ethyl acetate (200 mL) and dried the salt under vacuum and it was recrystallized from ethyl acetate for 10 times to give a pure S(−) or R(+) diastereomeric salts arising from S(+) phenyl glycenol and R(−) phenyl glycenol salts respectively. Which was treated (stirred) with 2N HCl (50 mL) at room temperature for 1 h. and filtered off the free acid from the salt through buchner funnel and washed the precipitate with DM-water and dried under vacuum at 50° C. for 12 hours to get a white pure S(−) and R(+) enantiomers (1.2 g each). R (+) enantiomer: M.P: 128-130; [α]²⁵ (EtOAc, C=0.5%)=+10.8 deg; Chiral purity=95.3 (by HPLC); S(−) enantiomer: M.P. 118-120° C.; [α]²⁵ (MeOH, C=0.5%)=−6.0 deg.

R(+): ¹H NMR(CD₃OD, 200 MHz): 7.70-7.29 (m, 14H); 6.91 (d, J=8.79 Hz, 2H); 6.83 (d, J=8.79 Hz, 2H); 6.08 (t, J=6.35 Hz, 1H); 4.71 (d, J=6.35 Hz, 2H); 4.29 (dd, 4.40 Hz, 8.31 Hz, 1H); 3.85 (dd, J=4.40 Hz, 11.23 Hz, 1H); 3.76 (dd, J=8.31 Hz, 11.72 Hz, 1H); 2.14 (s, 3H); 1.92-1.85 (m, 2H); 1.36 (s, 3H); 0.98 (t, J=7.32 Hz, 3H). IR(Cm⁻¹): 3443, 1567, 1505. Mass (electro spray): 554.8(M⁺+1).

S(−): ¹H NMR(CD₃OD, 200 MHz): 7.70-7.29 (m, 14H); 6.91 (d, J=8.79 Hz, 2H); 6.83 (d, J=8.79 Hz, 2H); 6.08 (t, J=6.35 Hz, 1H); 4.71 (d, J=6.35 Hz, 2H); 4.29 (dd, 4.40 Hz, 8.31 Hz, 1H); 3.85 (dd, J=4.40 Hz, 11.23 Hz, 1H); 3.76 (dd, J=8.31 Hz, 1.1.72 Hz, 1H); 2.14 (s, 3H); 1.92-1.85 (m, 2H); 1.36 (s, 3H); 0.98 (t, J=7.32 Hz, 3H). IR(Cm⁻¹): 3443, 1567, 1505. Mass (electro spray): 554.8(M⁺+1).

Solid-State Structure of R(+)-Isomer of Example-50:

The absolute stereo chemistry of R(+)-isomer of example-50, has been determined by single crystal studies. Single crystals suitable for X-ray diffraction have been grown from a mixture of methanol and ethyl acetate. The compound crystallizes in monoclinic space group P21 (#4), with cell dimensions a=12.14(8), b=6.35(2), c=19.71(6) Å, β=91.01(2)°, and V=1519.4(11) Å3 and Z=2. The calculated density is 1.21 g/cm3.

The intensity data have been collected on Rigaku AFC-7S single crystal Diffiactometer using Mo Kα radiation (λ=0.7107) on a CCD area-detector. The structure has been solved by direct methods (SIR92) and refined using least squares procedures with the Crystal Structure software. The present R factors are: R (RW)=0.036(0.041). There are 3904 unique reflections out of 19982 processed reflections. All the bond parameters are normal. The absolute stereo chemistry of R(+)-isomer of example-50, has been determined to be ‘R’ with respect to the configuration of (R)-2-Phenyl glycinol.

The ORTEP diagram is shown in the FIG. 1. Lists of interatomic distances and angles are given in Tables 1 and 2, respectively.

TABLE 1
Interatomic distances (Å)

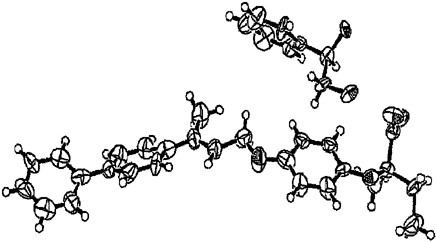

| Atom-Atom | Distance (Å) | Atom-Atom | Distance (Å) |
|---|---|---|---|
| O(1)-C(16) | 1.43 (1) | O(1)C(17) | 1.33 (2) |
| O(2)-C(20) | 1.36 (1) | O(2)-C(23) | 1.43 (1) |
| O(3)-C(24) | 1.25 (1) | O(4)-C(24) | 1.26 (1) |
| O(5)-C(28) | 1.41 (1) | N(1)-C(29) | 1.50 (1) |
| C(1)-C(2) | 1.41 (2) | C(1)-C(6) | 1.37 (2) |
| C(2)-C(3) | 1.37 (2) | C(3)-C(4) | 1.38 (2) |
| C(4)-C(5) | 1.34 (2) | C(5)-C(6) | 1.39 (1) |
| C(6)-C(7) | 1.47 (2) | C(7)-C(8) | 1.37 (1) |
| C(7)-C(12) | 1.36 (1) | C(8)-C(9) | 1.39 (2) |
| C(9)-C(10) | 1.37 (2) | C(10)-C(11) | 1.40 (1) |
| C(10)-C(13) | 1.45 (2) | C(11)-C(12) | 1.43 (2) |
| C(13)-C(14) | 1.50 (1) | C(13)-C(15) | 1.35 (2) |
| C(15)-C(16) | 1.48 (1) | C(17)-C(18) | 1.40 (1) |
| C(17)-C(22) | 1.46 (1) | C(18)-C(19) | 1.37 (1) |
| C(19)-C(20) | 1.40 (1) | C(20)-C(21) | 1.34 (1) |
| C(21)-C(22) | 1.37 (2) | C(23)-C(24) | 1.54 (1) |
| C(23)-C(25) | 1.51 (1) | C(23)-C(26) | 1.49 (1) |
| C(26)-C(27) | 1.50 (2) | C(28)-C(29) | 1.55 (1) |
| C(29)-C(30) | 1.49 (1) | C(30)-C(31) | 1.43 (2) |
| C(30)-C(35) | 1.38 (1) | C(31)-C(32) | 1.38 (2) |
| C(32)-C(33) | 1.35 (2) | C(33)-C(34) | 1.34 (2) |
| C(34)-C(35) | 1.38 (2) | | |

TABLE 2
Interatomic Angles (°))

| Atom—Atom—Atom | Angle(°)) | Atom—Atom—Atom | Angle(°) |
|---|---|---|---|
| C(16)—O(1)—C(17) | 116.0(7) | C(20)—O(2)—C(23) | 123.7(6) |
| C(2)—C(1)—C(6) | 124(1) | C(3)—C(2)—C(1) | 118(1) |
| C(4)—C(3)—C(2) | 118(1) | C(5)—C(4)—C(3) | 118(1) |
| C(6)—C(5)—C(4) | 127(1) | C(7)—C(6)—C(1) | 121.2(9) |
| C(7)—C(6)—C(5) | 126(1) | C(1)—C(6)—C(5) | 111(1) |
| C(8)—C(7)—C(12) | 116(1) | C(8)—C(7)—C(6) | 122.8(9) |
| C(12)—C(7)—C(6) | 120.3(8) | C(9)—C(8)—C(7) | 122(1) |
| C(10)—C(9)—C(8) | 123(1) | C(11)—C(10)—C(13) | 122(1) |
| C(11)—C(10)—C(9) | 113(1) | C(13)—C(10)—C(9) | 124.7(9) |
| C(12)—C(11)—C(10) | 123(1) | C(7)—C(12)—C(11) | 120.4(9) |
| C(14)—C(13)—C(15) | 119(1) | C(14)—C(13)—C(10) | 117.3(9) |
| C(15)—C(13)—C(10) | 123.3(9) | C(16)—C(15)—C(13) | 125.5(9) |
| O(1)—C(16)—C(15) | 105.6(7) | C(18)—C(17)—C(22) | 116(1) |
| C(18)—C(17)—O(1) | 129.4(9) | C(22)—C(17)—O(1) | 114.0(8) |
| C(19)—C(18)—C(17) | 122.0(9) | C(20)—C(19)—C(18) | 121.5(9) |
| C(21)—C(20)—O(2) | 119.0(8) | C(21)—C(20)—C(19) | 116.5(9) |
| O(2)—C(20)—C(19) | 124.5(7) | C(22)—C(21)—C(20) | 126.6(9) |
| C(17)—C(22)—C(21) | 116.7(9) | C(24)—C(23)—C(25) | 111.5(7) |
| C(24)—C(23)—C(26) | 106.5(7) | C(24)—C(23)—O(2) | 110.5(7) |
| C(25)—C(23)—C(26) | 111.9(8) | C(25)—C(23)—O(2) | 111.5(7) |
| C(26)—C(23)—O(2) | 104.6(6) | O(3)—C(24)—O(4) | 125.0(9) |
| O(3)—C(24)—C(23) | 115.7(8) | O(4)—C(24)—C(23) | 119.2(8) |
| C(27)—C(26)—C(23) | 114.4(9) | C(29)—C(28)—O(5) | 112.3(8) |
| C(30)—C(29)—N(1) | 113.0(7) | C(30)—C(29)—C(28) | 111.9(8) |
| N(1)—C(29)—C(28) | 110.5(7) | C(31)—C(30)—C(35) | 115.2(9) |
| C(31)—C(30)—C(29) | 119.2(8) | C(35)—C(30)—C(29) | 125.4(9) |
| C(32)—C(31)—C(30) | 119.8(9) | C(33)—C(32)—C(31) | 120(1) |
| C(34)—C(33)—C(32) | 122(1) | C(35)—C(34)—C(33) | 117(1) |
| C(30)—C(35)—C(34) | 124(1) | | |

Following are a few representative examples covered under general formula (I) that can be prepared by the person skilled in the art by following the procedure as described for Example-50:

| | | | |
|---|---|---|---|
| C(24)—C(23)—C(26) | 106.5(7) | C(24)—C(23)—O(2) | 110.5(7) |
| C(25)—C(23)—C(26) | 111.9(8) | C(25)—C(23)—O(2) | 111.5(7) |
| C(26)—C(23)—O(2) | 104.6(6) | O(3)—C(24)—O(4) | 125.0(9) |
| O(3)—C(24)—C(23) | 115.7(8) | O(4)—C(24)—C(23) | 119.2(8) |
| C(27)—C(26)—C(23) | 114.4(9) | C(29)—C(28)—O(5) | 112.3(8) |
| C(30)—C(29)—N(1) | 113.0(7) | C(30)—C(29)—C(28) | 111.9(8) |
| N(1)—C(29)—C(28) | 110.5(7) | C(31)—C(30)—C(35) | 115.2(9) |
| C(31)—C(30)—C(29) | 119.2(8) | C(35)—C(30)—C(29) | 125.4(9) |
| C(32)—C(31)—C(30) | 119.8(9) | C(33)—C(32)—C(31) | 120(1) |
| C(34)—C(33)—C(32) | 122(1) | C(35)—C(34)—C(33) | 117(1) |
| C(30)—C(35)—C(34) | 124(1) | | |

Following are a few representative examples covered under general formula (I) that can be prepared by the person skilled in the art by following the procedure as described for Example-50:

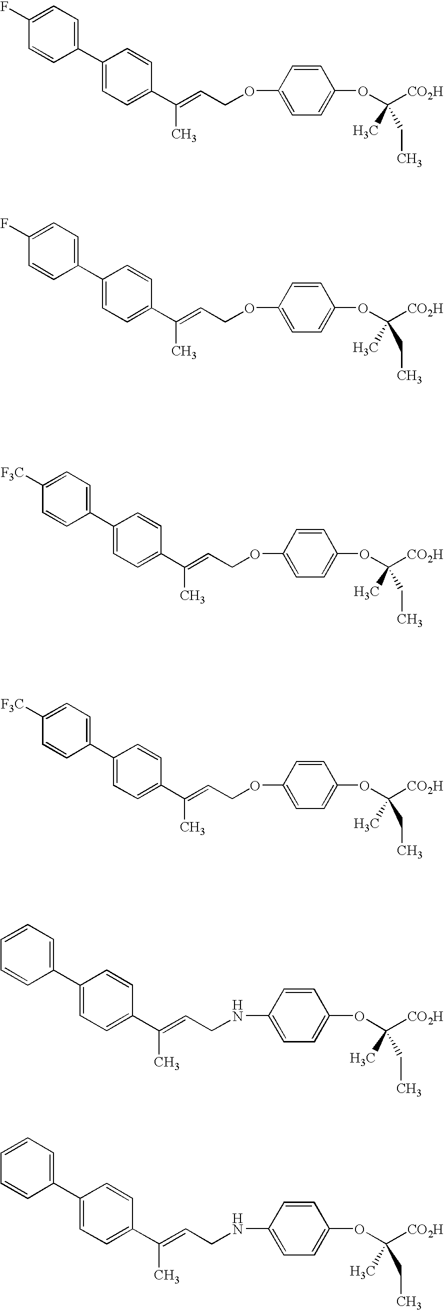

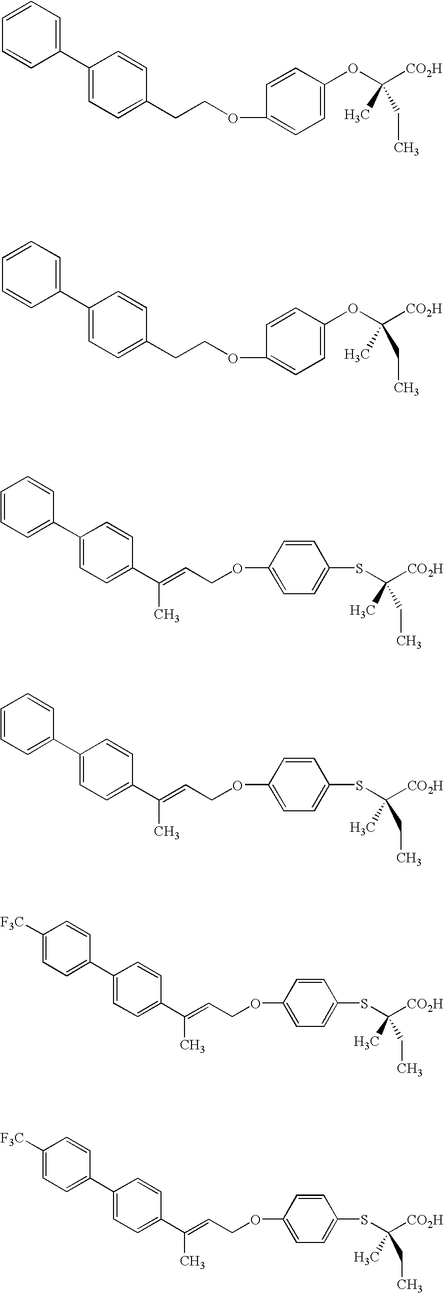

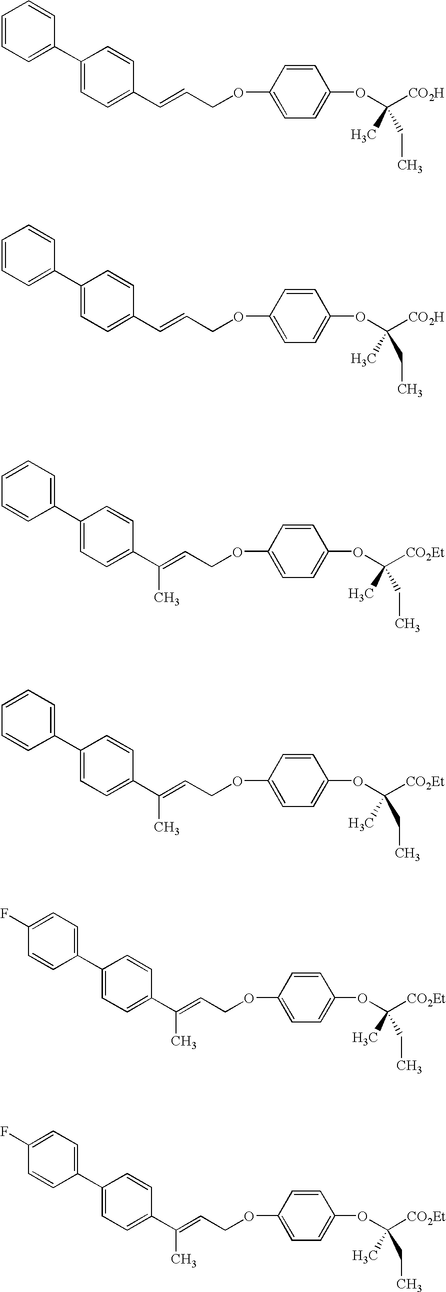

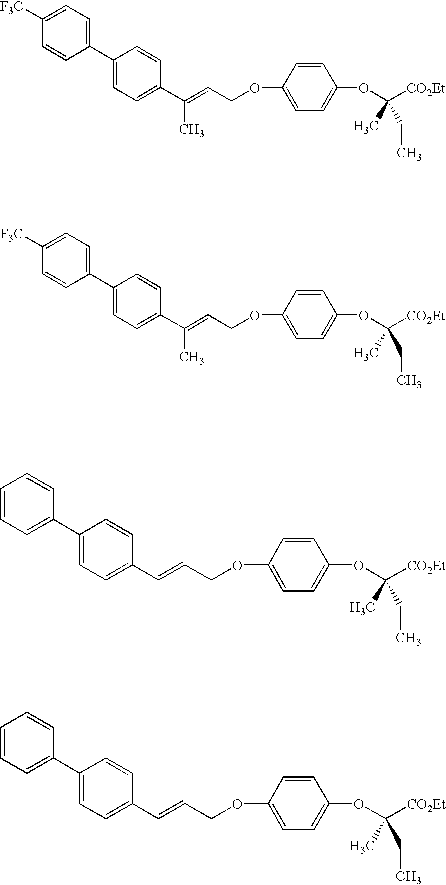

The compounds of the present invention lower triglyceride, total cholesterol, LDL, VLDL, random blood sugar level and increase HDL by agonistic mechanism. This may be demonstrated by in vitro as well as in vivo animal experiments

(A) In vitro:

(a) Determination of hPPARα activity:

Ligand binding domain of hPPARα was fused to DNA binding domain of Yeast transcription factor Gal 4 in eucaryotic expression vector. Using superfect (Qiagen, Germany) as transfecting reagent HEK-293 cells are transfected with this plasmid and a reporter plasmid harboring the luciferase gene driven by a GAL4 specific promoter. Compound can be added at different concentrations after 42 hrs of transfection and incubated overnight. Luciferase activity as a function of compound binding/activation capacity of PPARα will be measured using Packard Luclite kit (Packard, USA) in Top Count (Ivan Sadowski, Brendan Bell, Peter Broag and Melvyn Hollis. Gene. 1992. 118: 137-141; Superfect Transfection Reagent Handbook. February 1997. Qiagen, Germany).

(b) Determination of hPPARγ Activity

Ligand binding domain of hPPARγ1 is fused to DNA binding domain of Yeast transcription factor GAL4 in eucaryotic expression vector. Using lipofectamine (Gibco BRL, USA) as transfecting reagent HEK-293 cells are transfected with this plasmid and a reporter plasmid harboring the luciferase gene driven by a GAL4 specific promoter. Compound can be added at 1 μM. concentration after 48 hrs of transfection and incubated overnight Luciferase activity as a function of drug binding/activation capacity of PPARγ1 will be measured using Packard Luclite kit (Packard, USA) in Packard Top Count (Ivan Sadowski, Brendan Bell, Peter Broag and Melvyn Hollis. Gene. 1992. 118:137-141; Guide to Eukaryotic Transfections with Cationic Lipid Reagents. Life Technologies, GIBCO BRL, USA).

| Example | | | | |
|---|---|---|---|---|
| No | Concentration(μM) | PPARα | Concentration(μM) | PPARγ |
| Example | 50 | 12.2 | 1 | 2.8 |
| 37 | | | | |
| Example | 50 | 12.6 | 1 | 1.3 |
| 40 | | | | |

(c) Determination of HMG CoA Reductase Inhibition Activity

Liver microsome bound reductase is prepared from 2% cholestyramine fed rats at mid-dark cycle. Spectrophotometric assays are carried out in 100 mM KH₂PO₄, 4 mM DTT, 0.2 mM NADPH, 0.3 mM HMG CoA and 125 μg of liver microsomal enzyme. Total reaction mixture volume was kept as 1 ml. Reaction was started by addition of HMG CoA. Reaction mixture is incubated at 37° C. for 30 min and decrease in absorbance at 340 nm was recorded. Reaction mixture without substrate was used as blank (Goldstein, J. L and Brown, M. S. Progress in understanding the LDL receptor and HMG CoA reductase, two membrane proteins that regulate the plasma cholesterol. J. Lipid Res. 1984, 25: 1450-1461). The test compounds will inhibited the HMG CoA reductase enzyme.

(B) In Vivo

(a) Efficacy in Genetic Models

Mutation in colonies of laboratory animals and different sensitivities to dietary regimens has made the development of animal models with non-insulin dependent diabetes and hyperlipidemia associated with obesity and insulin resistance possible. Genetic models such as db/db and ob/ob (Diabetes, (1982) 31(1): 1-6) mice and zucker fa/fa rats have been developed by the various laboratories for understanding the pathophysiology of disease and testing the efficacy of new antidiabetic compounds (Diabetes, (1983) 32: 830-838; Annu. Rep. Sankyo Res. Lab. (1994). 46: 1-57). The homozygous animals, C57 BL/KsJ-db/db mice developed by Jackson Laboratory, US, are obese, hyperglycemic, hyperinsulinemic and insulin resistant (J. Clin. Invest., (1990) 85: 962-967), whereas heterozygous are lean and normoglycemic. In db/db model, mouse progressively develops insulinopenia with age, a feature commonly observed in late stages of human type II diabetes when blood sugar levels are insufficiently controlled. The state of pancreas and its course vary according to the models. Since this model resembles that of type II diabetes mellitus, the compounds of the present invention will be tested for blood sugar and triglycerides lowering activities.

Male C57BL/Ks-db/db mice of 8 to 14 weeks age, having body weight range of 35 to 60 grams, bred at Dr. Reddy's Research Foundation (DRF) animal house, were used in the experiment. The mice are provided with standard feed (National Institute of Nutrition (NIN), Hyderabad, India) and acidified water, ad libitum. The animals having more than 350 mg/dl blood sugar will be used for testing. The number of animals in each group will be 4.

Test compounds are suspended on 0.25% carboxymethyl cellulose and administered to test group at a dose of 0.1 mg to 30 mg/kg through oral gavage daily for 6 days. The control group receives vehicle (dose 10 ml/kg). On 6th day the blood samples will be collected one hour after administration of test compounds/vehicle for assessing the biological activity.

The random blood sugar and triglyceride levels can be measured by collecting blood (100 μl) through orbital sinus, using heparinised capillary in tubes containing EDTA which was centrifuged to obtain plasma. The plasma glucose and triglyceride levels can be measured spectrometrically, by glucose oxidase and glycerol-3-PO₄ oxidase/peroxidase enzyme (Dr. Reddy's Lab. Diagnostic Division Kits, Hyderabad, India) methods respectively.

The blood sugar and triglycerides lowering activities of the test compound are calculated according to the formula.

| | | Triglyceride |
|---|---|---|
| Compound | Dose (mg/kg) | Lowering (%) |
| Example 37 | 1 | 52 |

(b) Plasma Triglyceride and Cholesterol Lowering Activity in Hypercholesterolemic Rat Models

Male Sprague Dawley rats (NIN stock) were bred in DRF animal house. Animals were maintained under 12 hour light and dark cycle at 25±1° C. Rats of 180-200 gram body weight range were used for the experiment. Animals are made hypercholesterolemic by feeding 2% cholesterol and 1% sodium cholate mixed with standard laboratory chow [National Institute of Nutrition (NE), Hyderabad, India] for 6 days. Throughout the experimental period the animals were maintained on the same diet (Petit, D., Bonnefis, M. T., Rey, C and Infante, P Effects of ciprofibrate on liver lipids and lipoprotein synthesis in normo- and hyperlipidemic rats. Atherosclerosis. 1988. 74: 215-225).

The test compounds can be administered orally at a dose 0.1 to 30 mg/kg/day for 3 days. Control group was treated with vehicle alone (0.25% Carboxymethylcellulose; dose 10 ml/kg).

The blood samples can be collected in fed state 1 hour after drug administration on 0 and 3 day of compound treatment The blood can be collected from the retro-orbital sinus through heparinised capillary in EDTA containing tubes. After centrifugation, plasma sample will be separated for total cholesterol, HDL and triglyceride estimations. Measurement of plasma triglyceride, total cholesterol and HDL are were done using commercial kits (Dr. Reddy's Laboratory, Diagnostic Division, India). LDL and VLDL cholesterol can be calculated from the data obtained for total cholesterol, HDL and triglyceride. The reduction of various parameters examined are calculated according to the formula.

| | | Reduction in | | Increase | Reduction |
|---|---|---|---|---|---|
| | | Total | Triglyceride | in High Density | in Low Density |
| | Dose | Cholesterol | Lowering | Lipoprotien | Lipoprotien |
| Compound | (mg/kg) | (%) | (%) | (%) | (%) |
| Example 37 | 1 | 60 | 55 | 70 | 64 |

(c) Plasma Triglyceride and Total Cholesterol Lowering Activity in Swiss Albino Mice

Male Swiss albino mice (SAM) were obtained from NIN and housed in DRF animal house. All these animals are maintained under 12 hour light and dark cycle at 25±1° C. Animals are given standard laboratory chow (NIN, Hyderabad, India) and water, ad libitum. SAM of 20-25 g body weight range and Guinea pigs of 500-700 g body weight range are used (Oliver, P., Plancke, M. O., Marzin, D., Clavey, V., Sauzieres, J and Fruchart, J. C. Effects of fenofibrate, gemfibrozil and nicotinic acid on plasma lipoprotein levels in normal and hyperlipidemic mice. Atherosclerosis. 1988. 70: 107-114).

The test compounds can be administered orally to Swiss albino mice at 0.3 to 30 mg/kg/day dose for 6 days. Control mice are treated with vehicle (0.25% Carboxymethylcellulose; dose 10 ml/kg). The test compounds are administered orally to Guinea pigs at 0.3 to 30 mg/kg/day dose for 6 days. Control animals are treated with vehicle (0.25% Carboxymethylcellulose; dose 5 ml/kg).

The blood samples can be collected in fed state 1 hour after drug administration on 0 and 6 day of treatment. The blood can be collected from the retro-orbital sinus through heparinised capillary in EDTA containing tubes. After centrifugation, plasma sample was separated for triglyceride (Wieland, O. Methods of Enzymatic analysis. Bergermeyer, H. O., Ed., 1963. 211-214; Trinder, P. Ann. Clin. Biochem. 1969. 6: 24-27). Measurement of plasma triglyceride is done using commercial kits (Dr. Reddy's Diagnostic Division, Hyderabad, India).

| Compound | Dose (mg/kg) | Triglyceride Lowering (%) |
|---|---|---|
| Example 37 | 3 | 71 |

(d) Body Weight Reducing Effect in Cholesterol Fed Hamsters:

Male Syrian Hamsters are procured from NIN, Hyderabad, India. Animals are housed at DRF animal house under 12 hour light and dark cycle at 25±1° C. with free access to food and water. Animals are maintained with 1% cholesterol containing standard laboratory chow (NIN) from the day of treatment.

The test compounds can be administered orally at 1 to 30 mg/kg/day dose for 15 days. Control group animals are treated with vehicle (Mill Q water, dose 10 ml/kg/day). Body weights are measured on every 3^{rd} day.

| | | Reduction | Reduction | |
|---|---|---|---|---|
| | | in Total | in | |
| | Dose | Cholesterol | Triglyceride | Reduction in |
| Compound | (mg/kg) | (%) | (%) | Body weight (%) |
| Example 25 | 3 | 55 | 45 | 22 |

Formulae for Calculation:
1. Percent reduction in Blood sugar triglycerides/total cholesterol will be calculated according to the formula:

$$Percent reduction ( \% ) = [ 1 - \frac{TT / OT}{TC / OC} ] \times 100$$

OC=Zero day control group value
OT=Zero day treated group value
TC=Test day control group value
TT=Test day treated group value
2. LDL and VLDL cholesterol levels will be calculated according to the formula:

$$LDL cholesterol in mg \text{/} dl = [ \begin{matrix}Total cholesterol - \\ HDL cholesterol - \\ \frac{Triglyceride}{5}\end{matrix} ] mg \text{/} dl$$

$$VLDL cholesterol in mg \text{/} dl = [ \begin{matrix}Total cholesterol - \\ HDL cholesterol - \\ LDL cholesterol\end{matrix} ] mg \text{/} dl .$$

We claim:

1. A compound of formula (I), 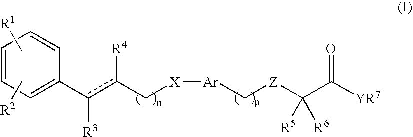
wherein R¹ and R² may be same or different and independently represent hydrogen, halogen, nitro, cyano, amino, hydroxy or optionally substituted group selected from alkyl, cycloalkyl, alkoxy, cycloalkoxy, aryl, aralkyl, alkylcarbonyl, alkoxycarbonyl, arylcarbonyl, alkoxycarbonyl, aralkoxycarbonyl, heteroarylcarbonyl, aryloxy, aralkoxy, alkylcarbonyloxy, alkoxycarbonylamino, aryloxycarbonylamino, aralkoxycarbonylamino, heteroarylcarbonylamino, heteroaryl, heteroaralkyl, heterocyclyl, heteroaralkoxy, heteroaryloxy, fluorenylmethoxycarbonyl (Fmoc), fluorenylmethoxycarbonylamino (N-Fmoc), —OSO₂R⁸, —OCONR⁸R⁹, NR⁸COOR⁹, —NR⁸COR⁹, —NR⁸R⁹, —NR⁸SO₂R⁹, —NR⁸CONR⁹R¹⁰, —NR⁸CSNR⁸R⁹, —SO₂R⁸, —SOR⁸, —SR⁸, —SO₂NR⁸R⁹, —SO₂OR⁸, —CONR⁸R⁹, —COOR⁹ or —COR⁹, wherein R⁸, R⁹ and R¹⁰ may be same or different and independently represent hydrogen, optionally substituted group selected from alkyl, aryl, aralkyl, aryloxy or heteroaryl; or R¹ and R² together represent a monocyclic or polycyclic aromatic or non aromatic ring or an aromatic ring fused to a non aromatic ring, which may optionally contain 1 to 3 heteroatoms selected from N, S, or O and may be unsubstituted or have 1 to 4 substituents which may be identical or different.
R³ and R⁴ may be same or different and independently represent hydrogen, halogen, optionally substituted group selected from alkyl, cycloalkyl, alkanoyl, aryl, aroyl, aralkyl or aralkanoyl group. ‘n’ and ‘p’ independently represents 0-6.
X represents O, S, NR where R represents hydrogen or optionally substituted groups selected from alkyl, cycloalkyl, cycloalkylalkyl, aryl, aralkyl, alkanoyl, or aroyl.
Ar represents optionally substituted single or fused aromatic, heteroaromatic or heterocyclic group.
Z represents O, S, NR where R is as defined above.
R⁵, R⁶ and R⁷ may be same or different and independently represent hydrogen, hydroxy, halogen or optionally substituted group selected from alkyl, cycloalkyl, alkoxy, aryl, aralkyl, heteroaryl, heterocyclyl or heteroaralkyl groups. R⁵ and R⁶ together may form a 5 or 6 membered cyclic rings, which may contain one or two hetero atoms selected from O, S or N.
Y represents O or NR¹¹ where R¹¹ represents hydrogen, optionally substituted group selected from alkyl, aryl, aralkyl, alkanoyl, aroyl, aralkanoyl, heterocyclyl or heteroaryl.
R⁷ and R¹¹ together may also form a 5 or 6 membered cyclic ring, which may contain one or two hetero atoms selected from O, S or N.
‘----’ represents a bond or no bond; their stereoisomers, pharmaceutically acceptable salts thereof as well as pharmaceutical compositions containing them;
When the fused rings formed by R¹ and R² are substituted, the substituents are selected from alkyl, halogen, hydroxy, haloalkyl, nitro, amino, cyano, oxo, or thioxo.
When the groups represented by R¹ and R² are substituted, the substituents are selected from halogen, hydroxy, nitro, amino, oxo, thioxo, optionally substituted groups selected from alkyl, cycloalkyl, alkoxy, aryl, aralkyl, alkylsulfonyl, alkylsulinyl, alkylsulfanyl, alkylsulfonyloxy, alkylsulfinyloxy or alkylsulfanyloxy, the substituents are selected from halogen, hydroxyl, nitro, amino, cyano or alkyl.
When the groups represented by R⁵, R³, R⁴ and R¹¹ are substituted, the substituents are selected from halogen, nitro, amino, hydroxy, alkyl, oxo or aralkyl
When the groups represented by R⁵, R⁶ and R⁷ are substituted, the substitutents are selected from halogen, hydroxy, nitro, alkyl, cycloalkyl, alkoxy, aryl, aralkyl, aralkoxyalkyl, heterocyclyl, heteroaryl or amino.
When the cyclic rings formed by R⁵ and R⁶ are substituted, the substituents are selected from alkyl, halogen, hydroxy, haloalkyl, nitro, amino, cyano, oxo, or thioxo.
The groups defined for R, R¹, R², R³, R⁴, R⁵, R⁶, R⁷, R⁸, R⁹, R¹⁰ and R¹¹ may be unsubstiuted, or have 1 to 4 substituents, which may be identical or different.

2. The compound of claim 1, wherein the stereoisomer is enantiomer and/or geometrical isomer.

3. The compound of claim 1 wherein ‘Ar’ is optionally substituted groups selected from phenylene, naphthylene, pyridyl, quinolinyl, benzofuryl, dihydrobenzofuryl, benzopyranyl, dihydrobenzopyranyl, indolyl, indolinyl, azaindolyl, azaindolinyl, pyrazolyl, benzothiazolyl or benzoxazolyl. The substituents on the group represented by ‘Ar ’ may be selected from linear or branched optionally halogenated (C₁-C₁₀)alkyl, optionally halogenated (C₁-C₁₀)alkoxy, halogen, acyl, amino, acylamino, thio or carboxylic or sulfonic acids and their derivatives, which may optionally be susbstituted.

4. The compound of claim 1 wherein ‘Ar’ is optionally substituted phenylene, naphthylene, benzofuryl, indolyl, indolinyl, quinolinyl, azaindolyl, azaindolinyl, benzothiazolyl or benzoxazolyl groups.

5. The compound of claim 1 wherein ‘Ar’ is phenylene, naphthylene or benzofuryl, which may be unsubstituted or substituted by alkyl, haloalkyl, methoxy or haloalkoxy groups.

6. The compound of claim 1 wherein
R¹ and R² are same or different and independently represent hydrogen, halogen, nitro, cyano, amino, hydroxy or optionally substituted groups selected from alkyl, alkoxy, aryl, aralkyl, aralkoxy, heteroaryl, heteroaralkoxy, —OSO₂R⁸, —SO₂R⁸ or —NR⁸R⁹;
R³ and R⁴ are same or different and independently represent hydrogen, halogen, optionally substituted group selected from alkyl or aralkyl;
R⁵ and R⁶ are same or different and independently represent hydrogen, hydroxy, optionally substituted alkyl, cycloalkyl, aryl or R⁵ and R⁶ together represent a 5 or 6 membered aromatic or non aromatic cyclic ring system optionally containing 1 or 2 heteroatoms selected from O, S or N;
R⁷ and R¹¹ may form a cyclic ring system selected from pyrrolidinyl, piperidinyl, morpholinyl, piperazinyl, oxazolinyl, diazolinyl and the like.

7. The compound of claim 1 wherein
R¹ and R² together represent a optionally susbstituted monocyclic or polycyclic aromatic or non aromatic ring or an aromatic ring fused to a non aromatic ring selected from:

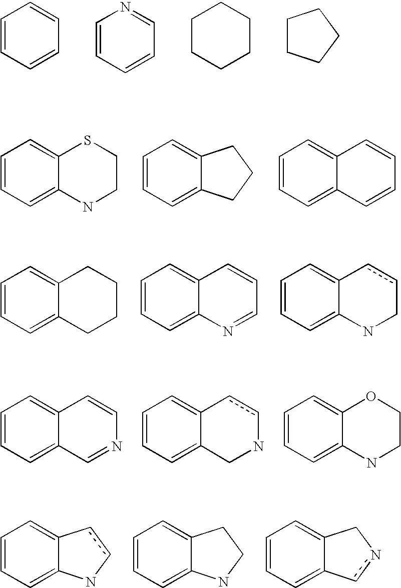

8. The compound of claim 1 wherein:
R₁ and R² are same or different and independently represent hydrogen, halogen, nitro, amino, hydroxy or optionally substituted group selected from alkyl, aryl, aralkyl, aralkoxy, heteroaryl, heteroaralkoxy or —OSO₂R⁸;
R³ and R⁴ are same or different and independently represent hydrogen or optionally substituted alkyl;
R⁵ and R⁶ are same or different and independently represent hydrogen, optionally substituted alkyl, cycloalkyl, aryl or R⁵ and R⁶ together represent a optionally substituted 5 or 6 membered saturated cyclic ring system

9. The compound of claim 1 wherein:
R¹ and R² together represent a optionally susbstituted monocyclic or polycyclic aromatic or non aromatic ring or an aromatic ring fused to a non aromatic ring selected from: 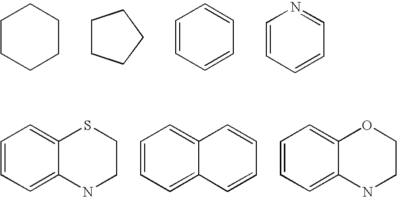
R³ and R⁴ are same or different and independently represent hydrogen or optionally substituted alkyl;
R⁵ and R⁶ are same or different and independently represent hydrogen, optionally substituted group selected from alkyl, cycloalkyl, aryl or R⁵ and R⁶ together represent a 5 or 6 membered saturated cyclic ring system;

10. The compound of claim 1 wherein
R¹ is selected from —SO₂CH₃, halogen, alkyl optionally substituted phenyl wherein the substituent is selected from alkyl or halogen
R², R³, R⁴, R⁵, R⁶ and R⁷ are same or different and independently represent hydrogen, methyl, ethyl or propyl
‘Ar’ represents optionally substituted phenyl wherein the substituent is alkyl X, Y and Z independently represent oxygen
n and p independently represent 0 or 1

11. The compound of claim 1 wherein
R¹ is selected from optionally substituted phenyl wherein the substituent is selected from halogen
R², R³, R⁴, R⁵, R⁶ and R⁷ are same or different and independently represent hydrogen, methyl, ethyl or propyl
‘Ar’ represents optionally substituted phenyl wherein the substituent is alkyl
X, Y and Z independently represent oxygen
n and p independently represent 0 or 1.

12. The compound of formula (I) as claimed in claim 1 is selected from:

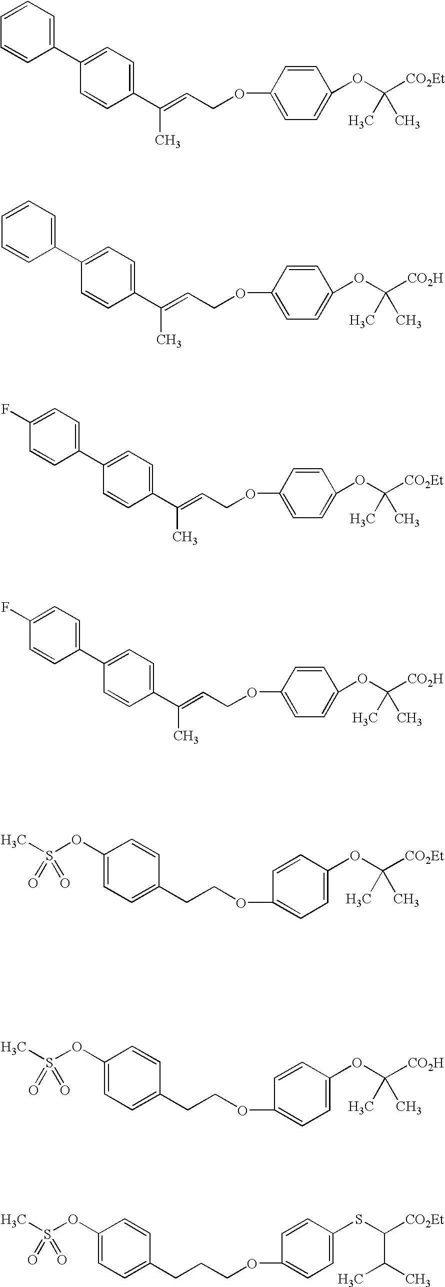

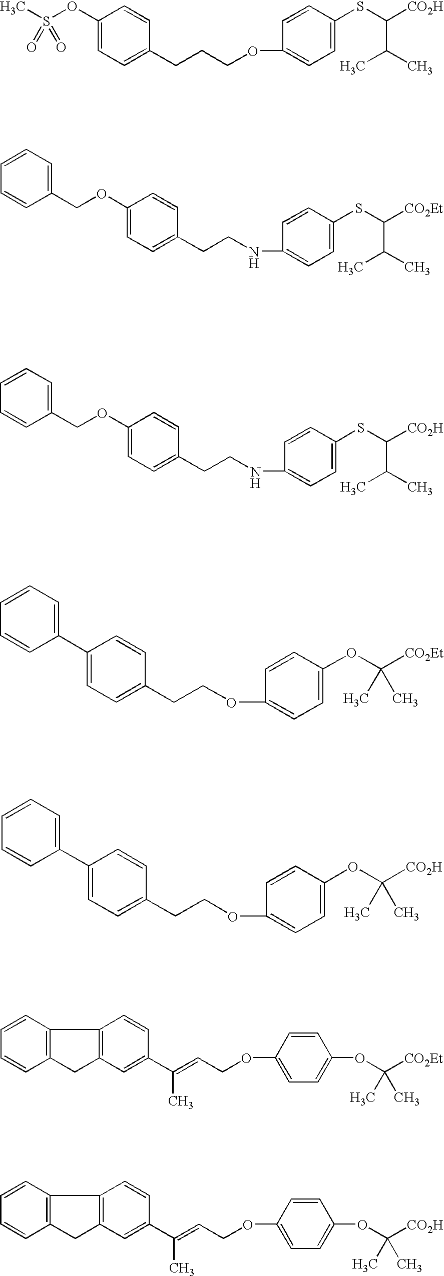

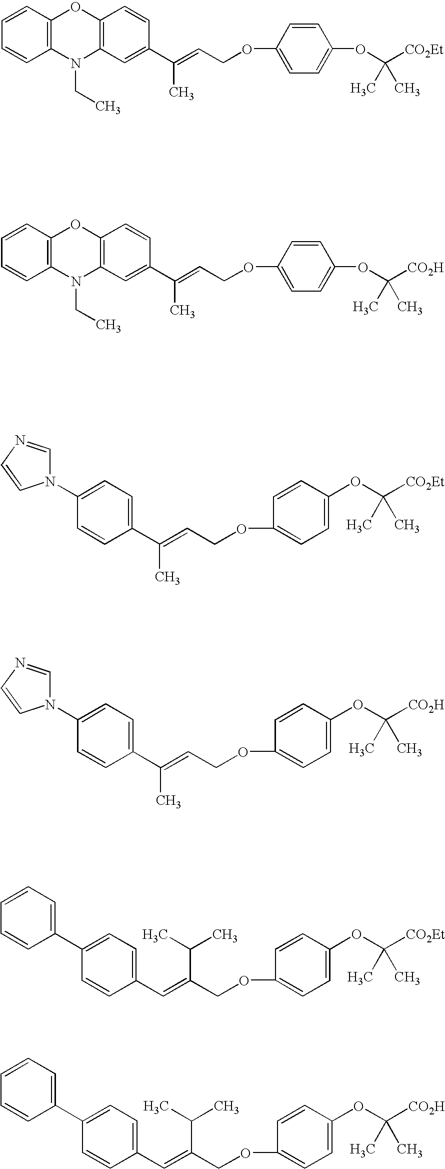

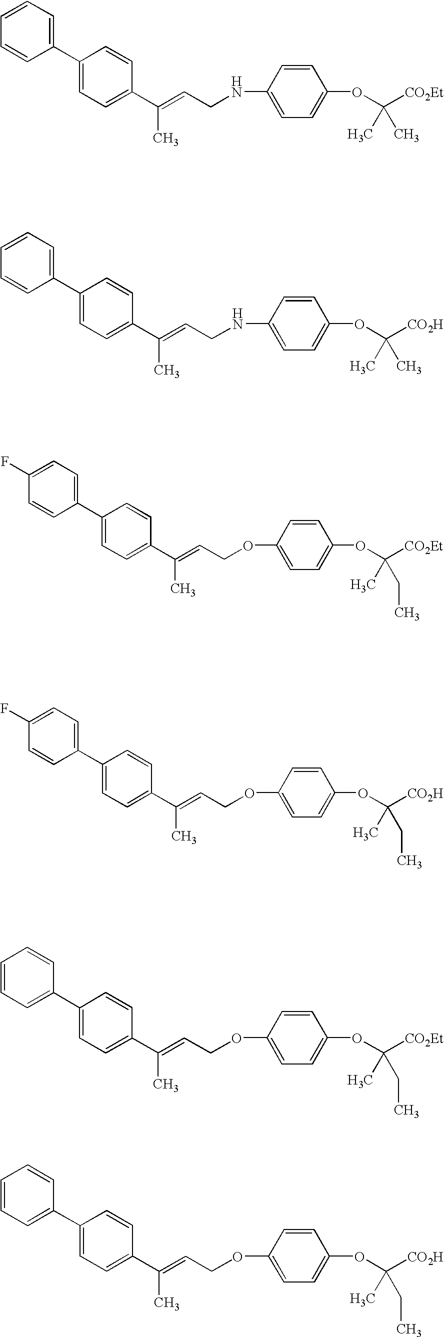

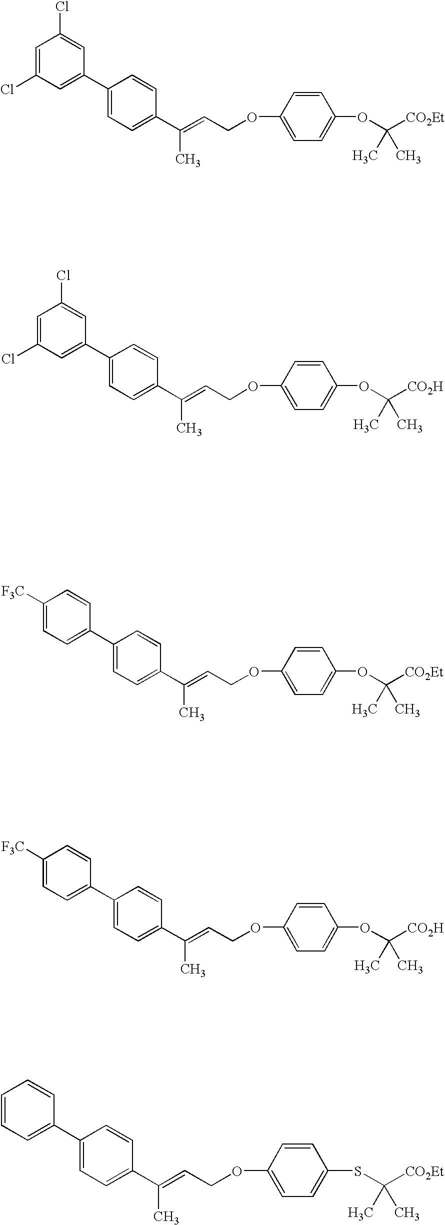

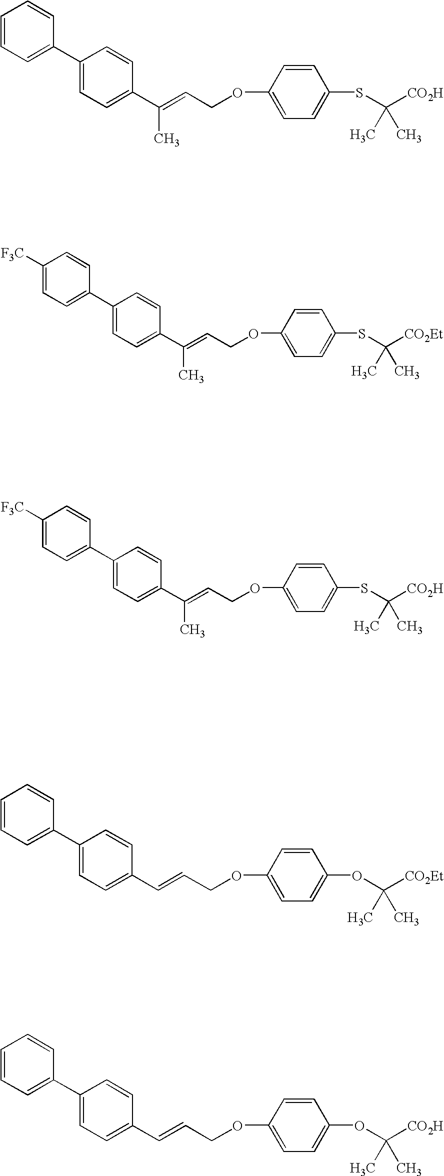

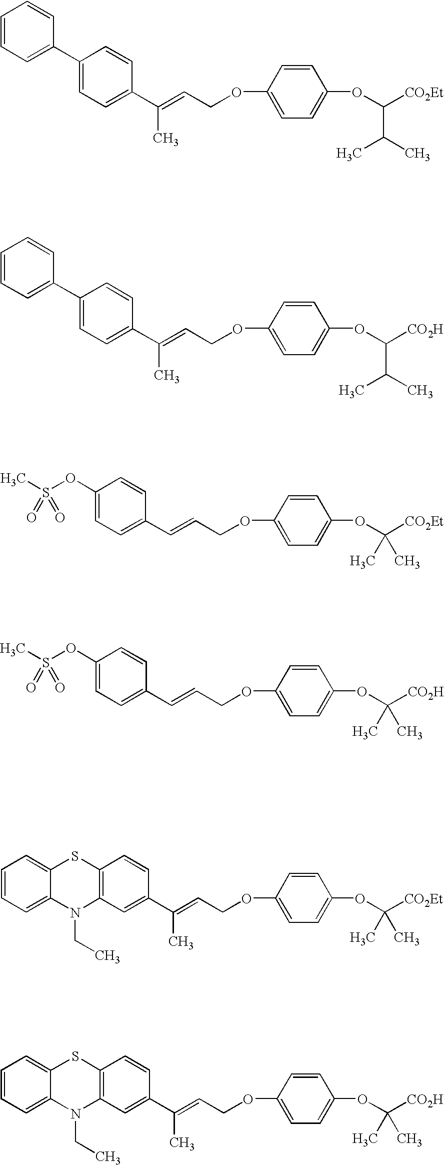

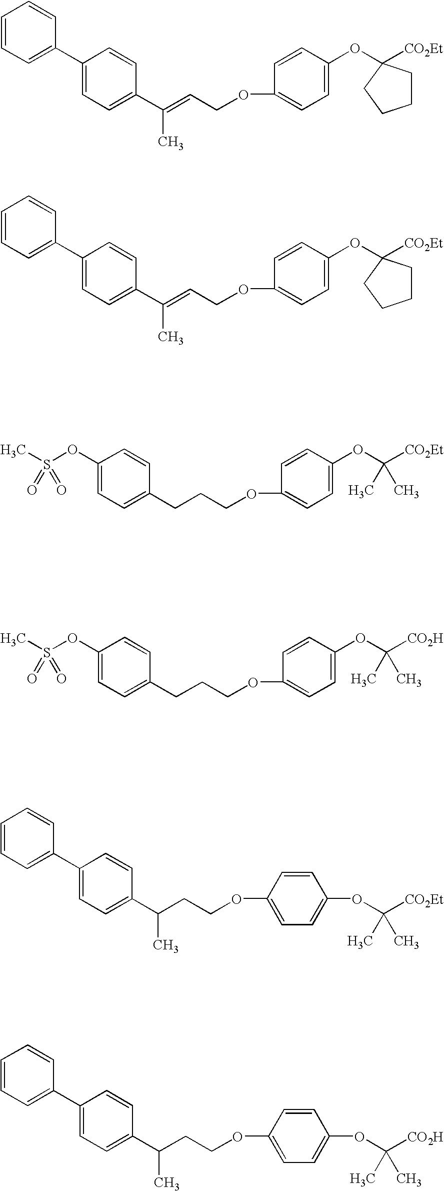

13. The compound of formula (I) as claimed in claim 1 is selected from:

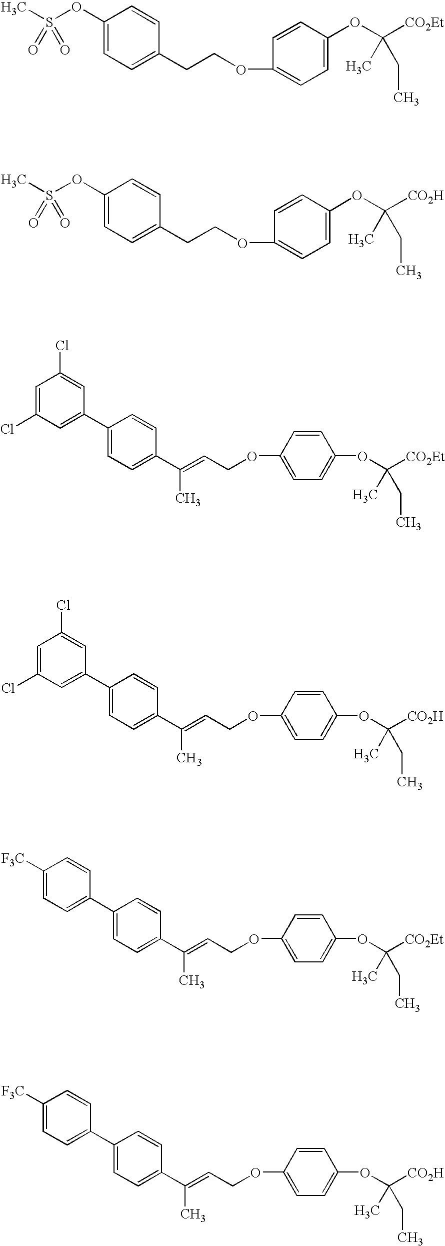

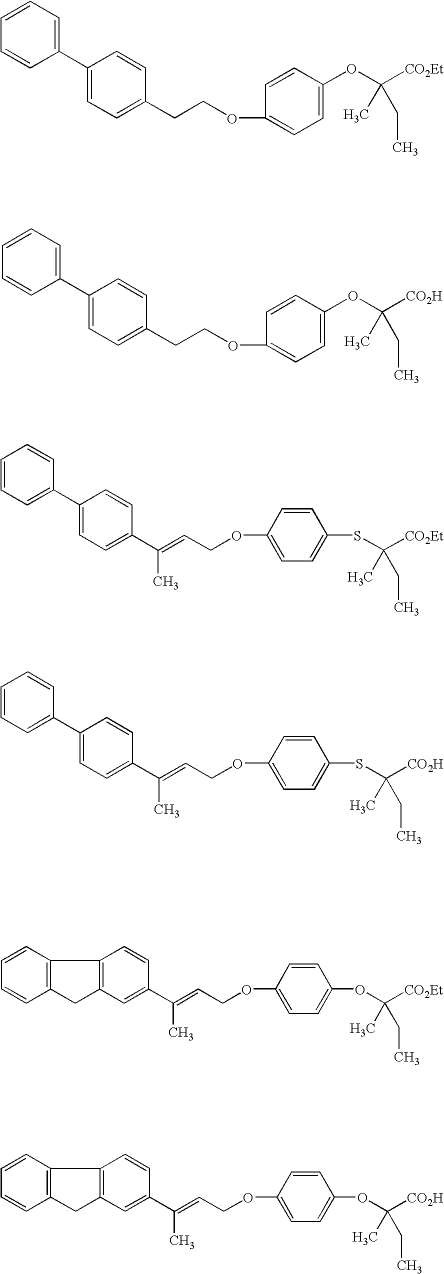

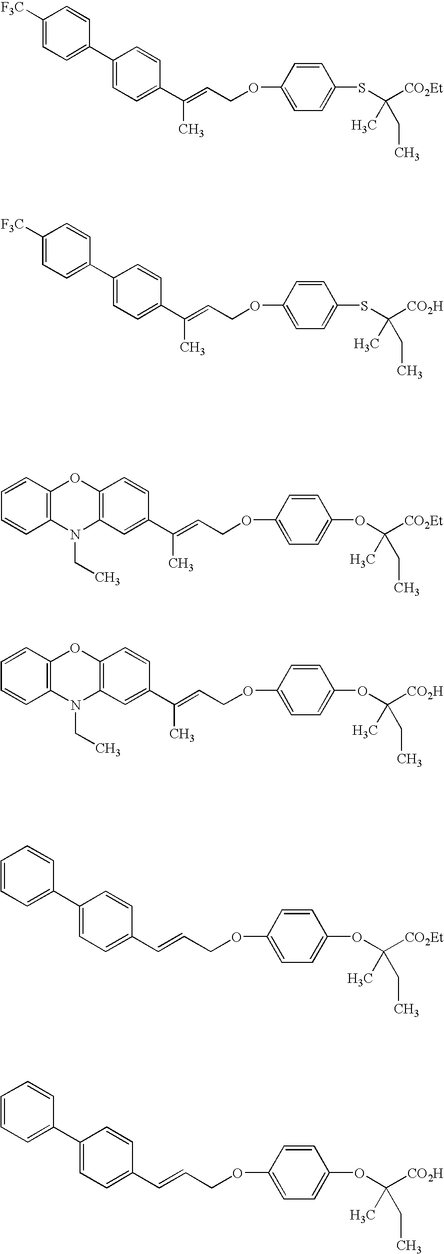

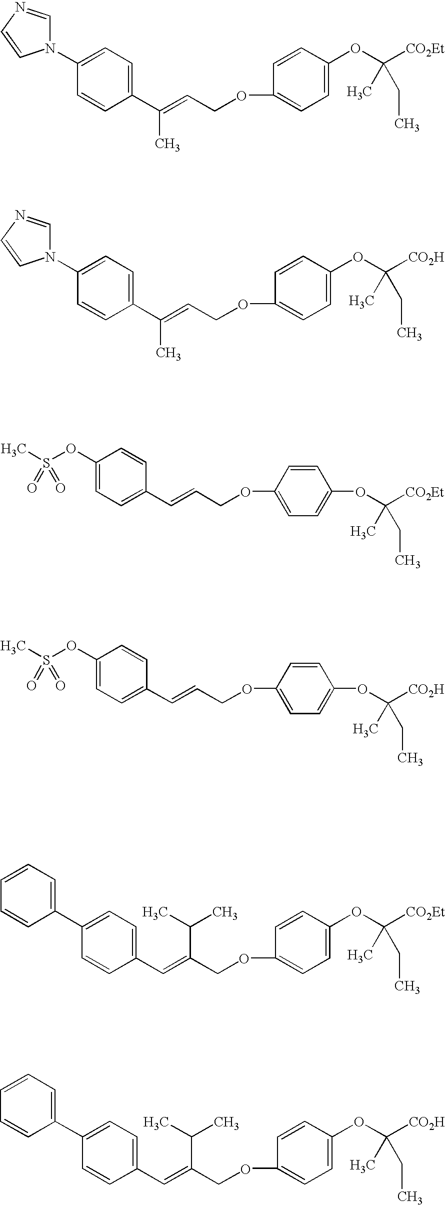

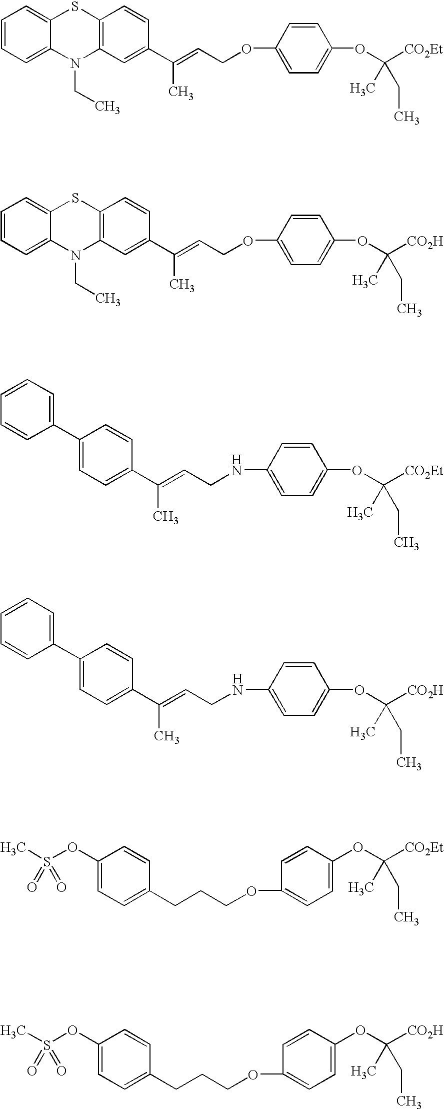

14. The compound of formula (I) as claimed in claim 1 is selected from:

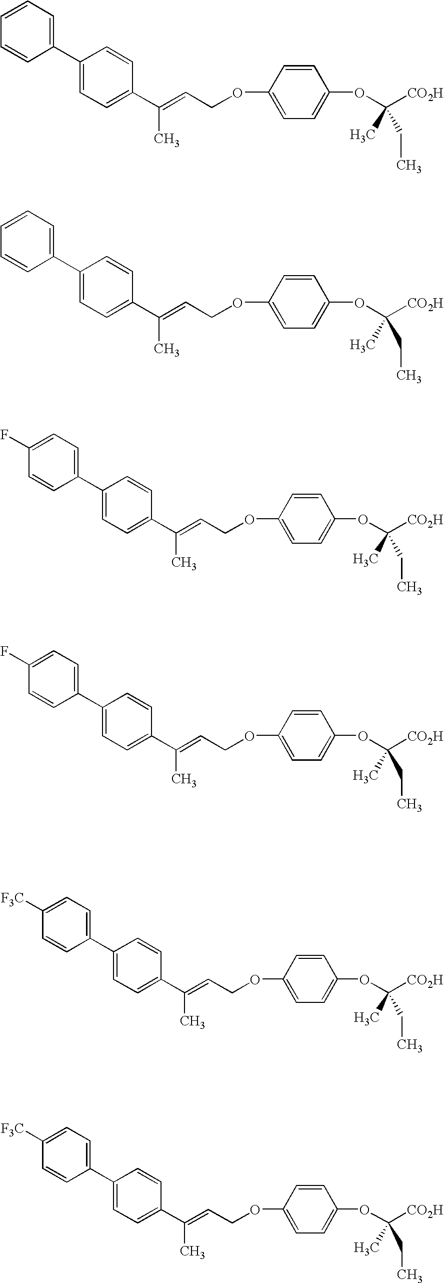

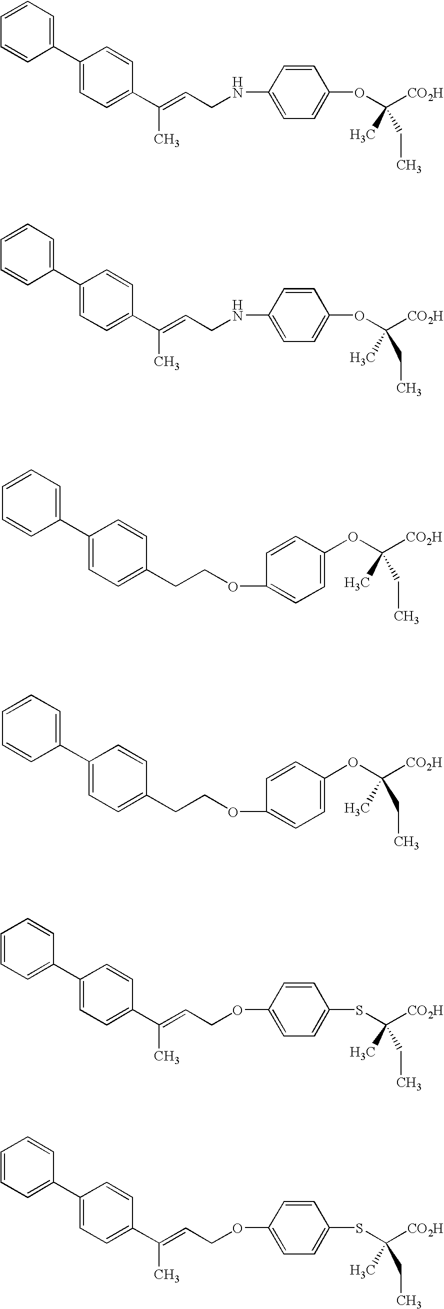

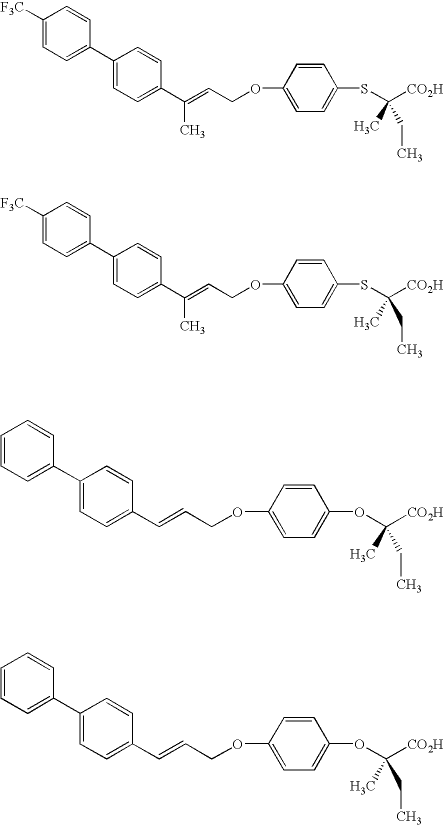

15. The compound of formula (I) as claimed in claim 1 is selected from:

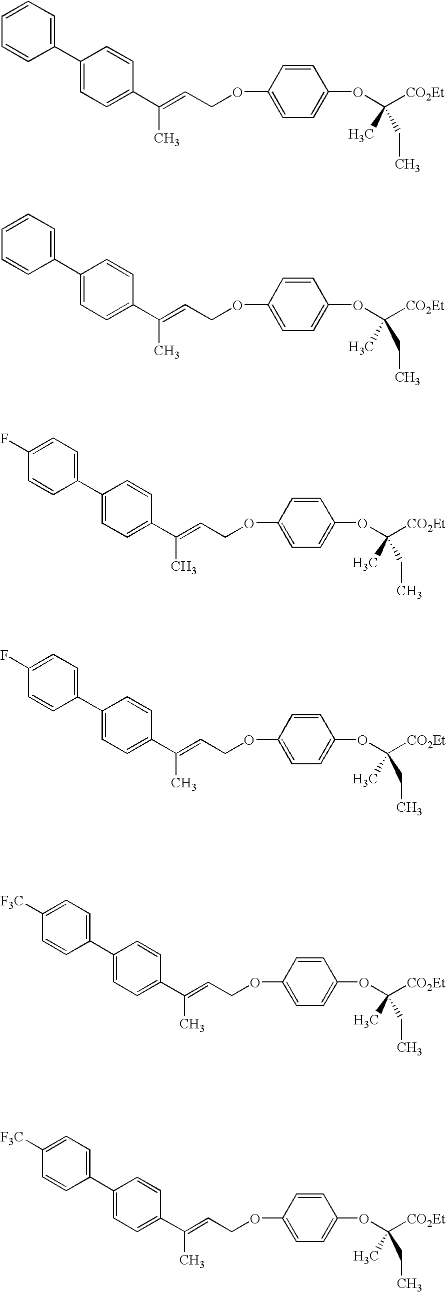

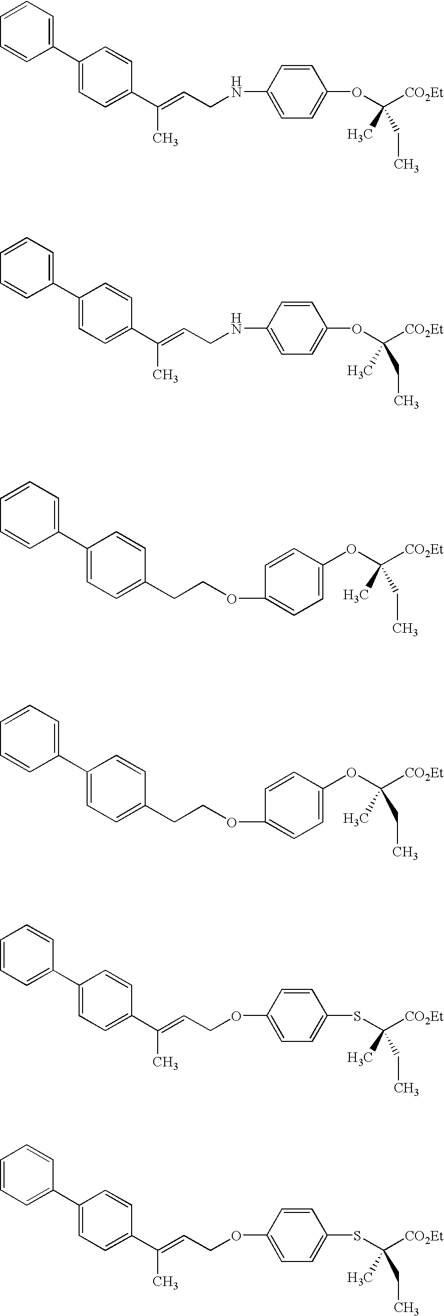

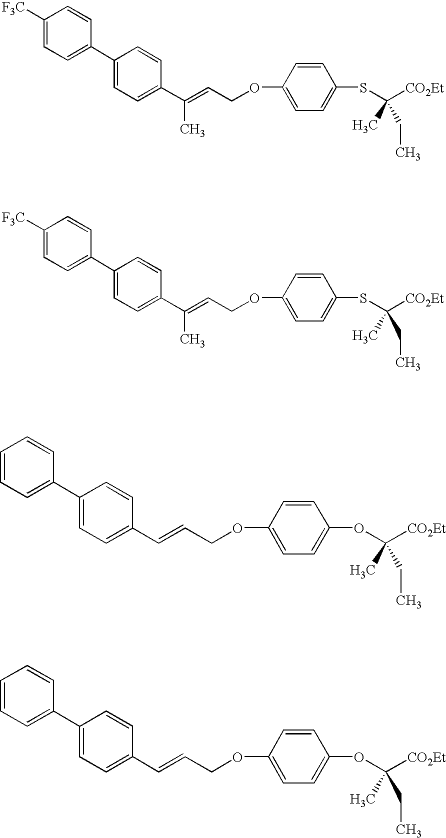

16. The compound, of formula (I) as claimed in claim 1 is selected from:

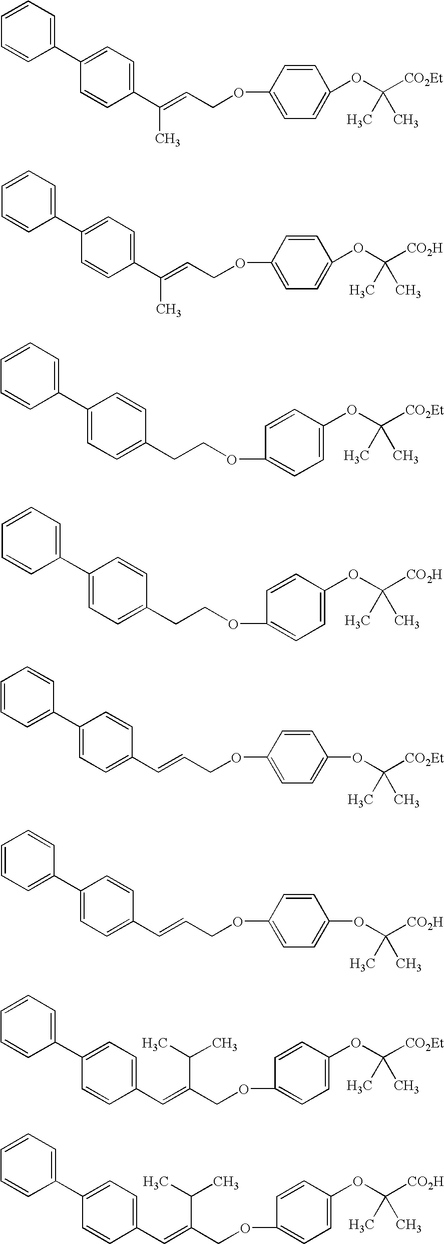

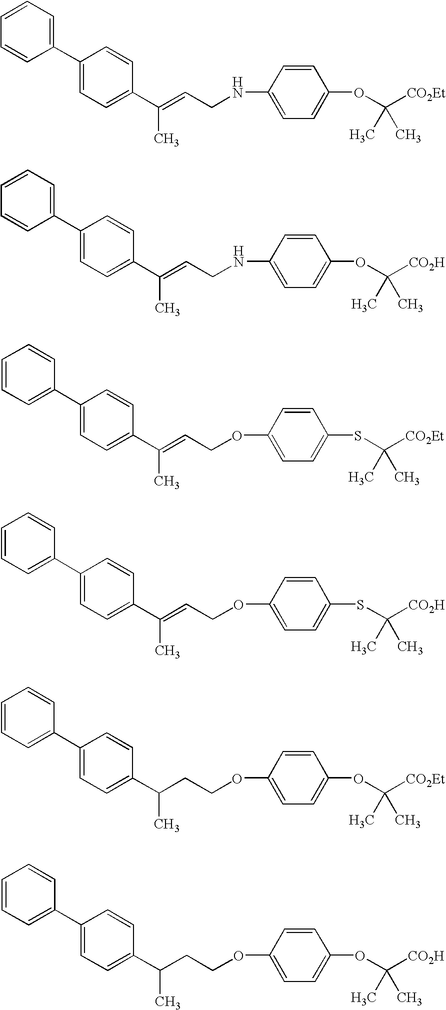

17. The compound of formula (I) as claimed in claim 1 is selected from:

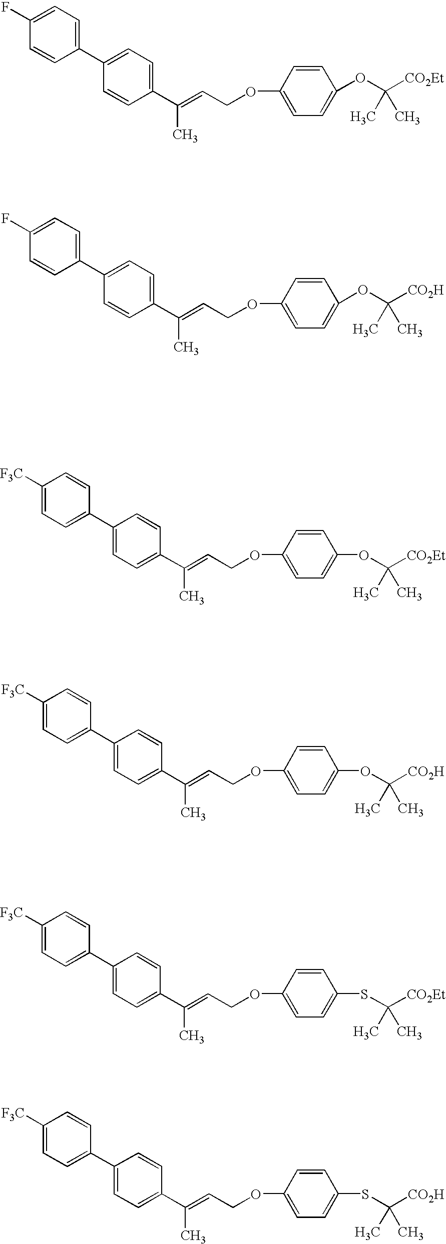

18. The compound of formula (I) as claimed in claim 1 is selected from:

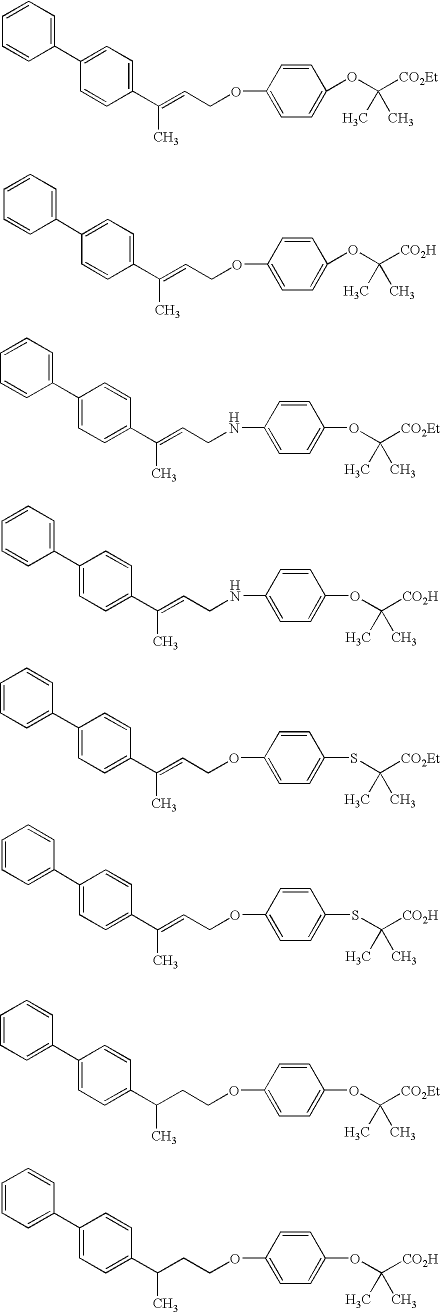

19. The compound of formula (I) as claimed in claim 1 is selected from:

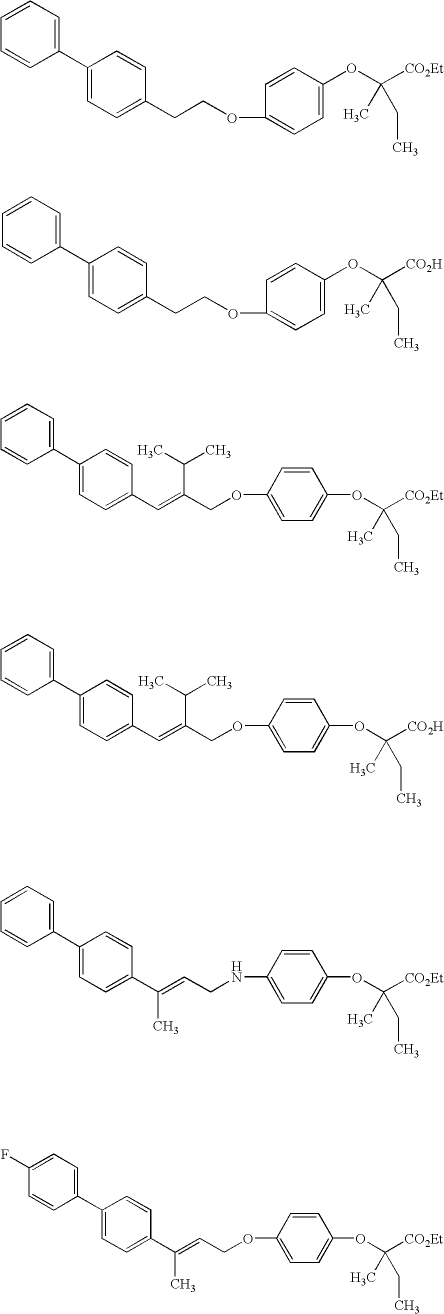

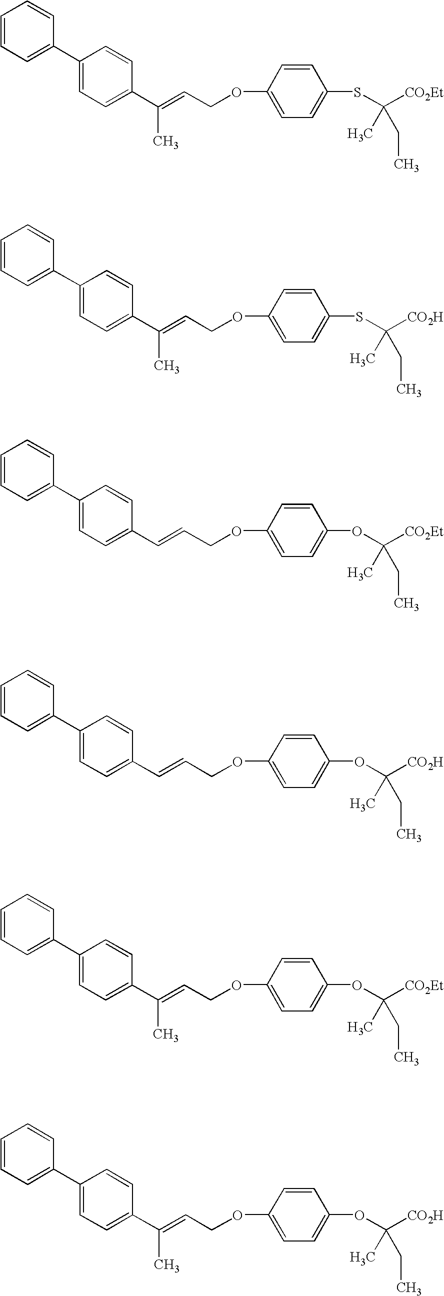

20. The compound of formula (I) as claimed in claim 1 is selected from:

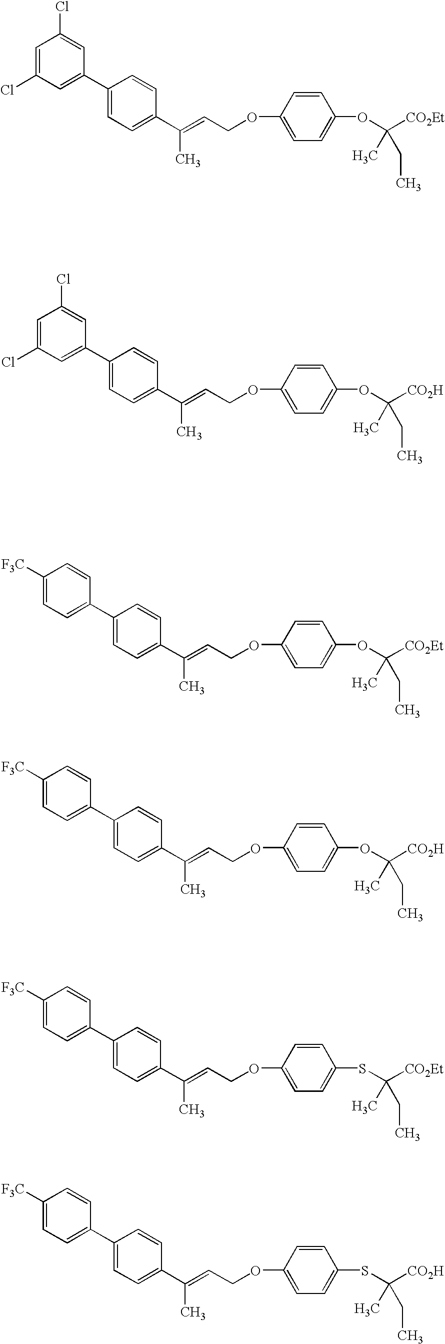

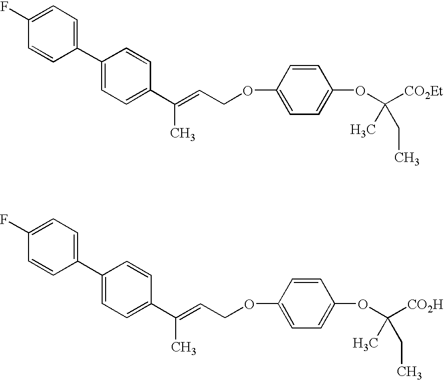

21. The compound of formula (I) as claimed in claim 1 is selected from:

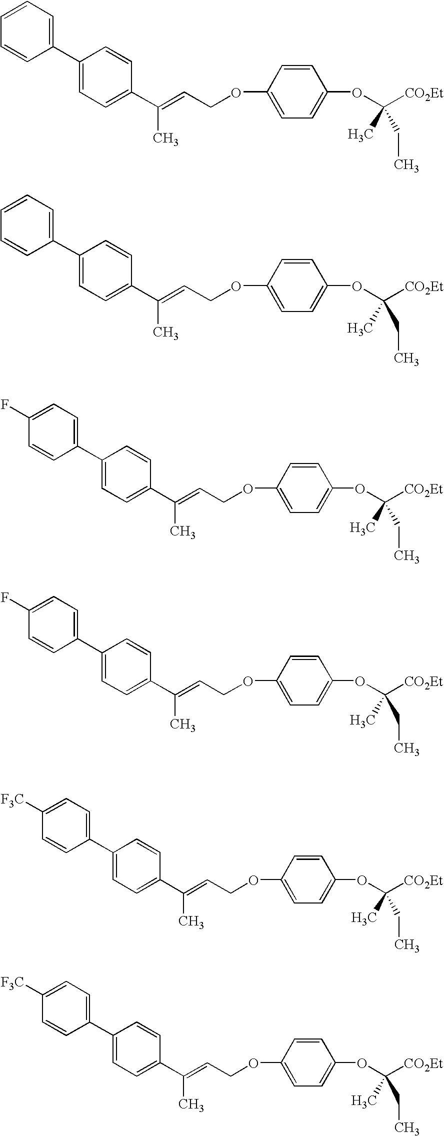

22. The compound of formula (I) as claimed in claim 1 is selected from:

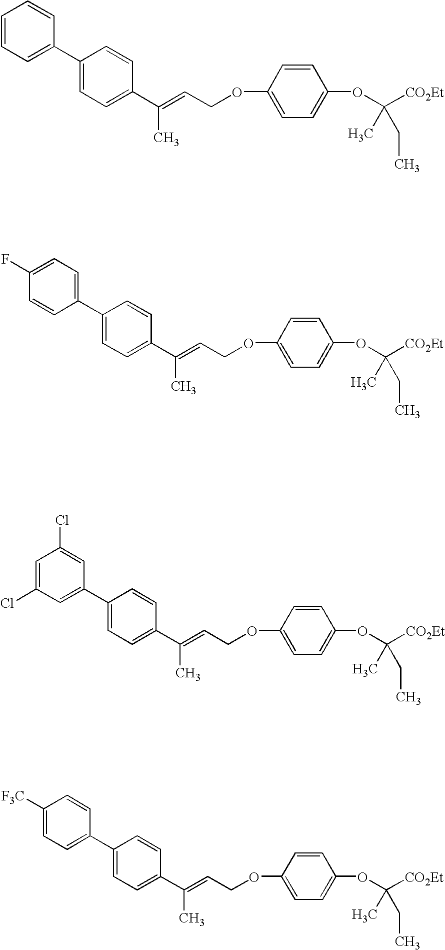

23. The compound of formula (I) as claimed in claim 1 is selected from:

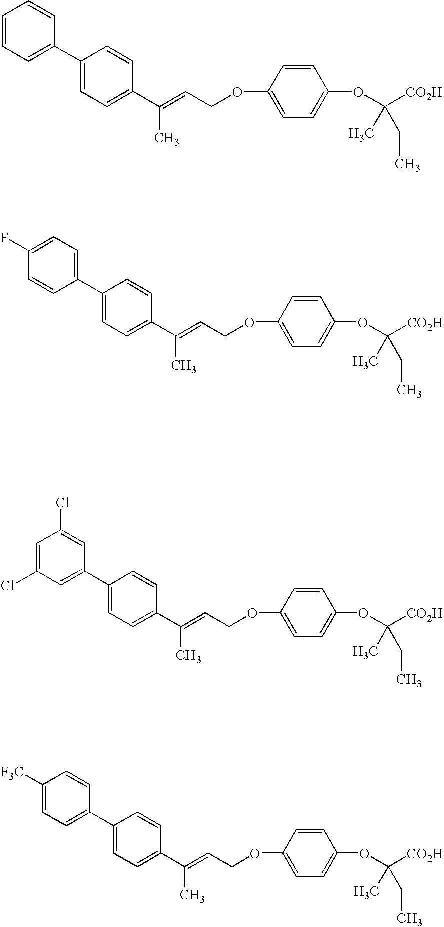

24. The compound of formula (I) as claimed in claim 1 is selected from:

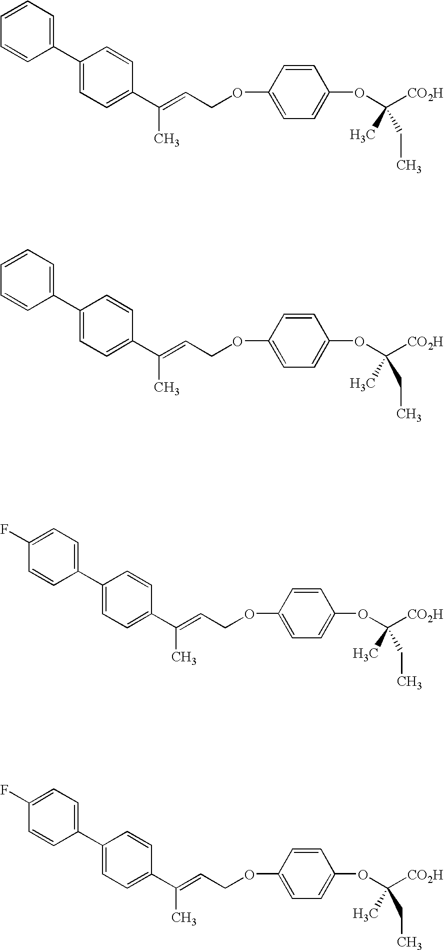

25. The compound of formula (I) as claimed in claim 1 is

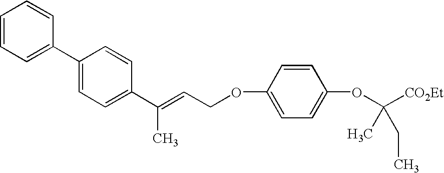

26. The compound of formula (I) as claimed in claim 1 is

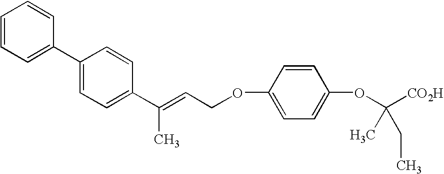

27. The compound of formula (I) as claimed in claim 1 is

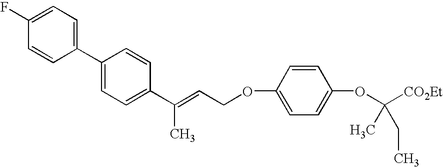

28. The compound of formula (I) as claimed in claim 1 is

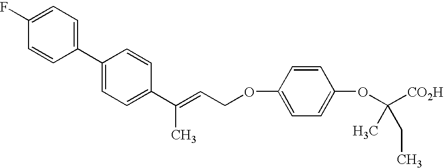

29. The compound of formula (I) as claimed in claim 1 is

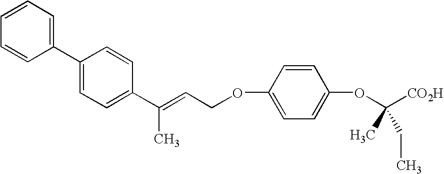

30. The compound of formula (I) as claimed in claim 1 is

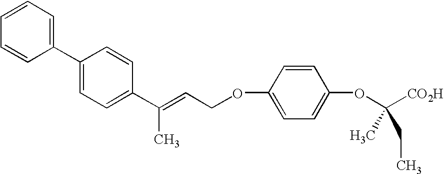

31. The compound of formula (I) as claimed in claim 1 is

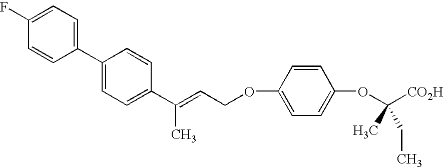

32. The compound of formula (I) as claimed in claim 1 is

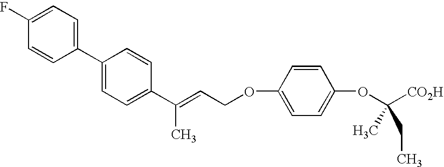

33. The compound of formula (I) as claimed in claim 1 is

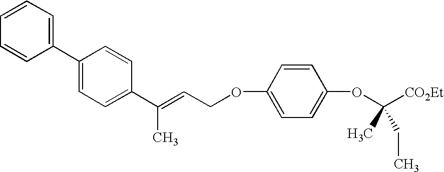

34. The compound of formula (I) as claimed in claim 1 is

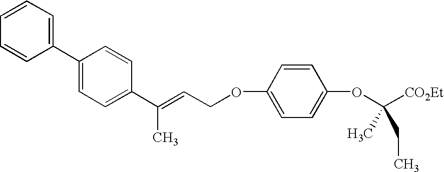

35. The compound of formula (i) as claimed in claim 1 is

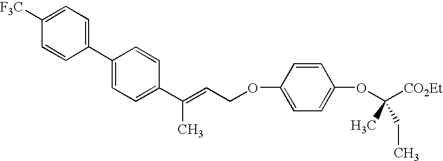

36. The compound of formula (I) as claimed in claim 1 is

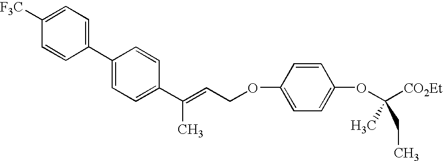

37. A process for the preparation of compound of formula (I) 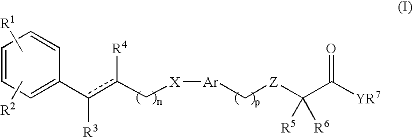
wherein R¹ and R² may be same or different and independently represent hydrogen, halogen, nitro, cyano, amino, hydroxy or optionally substituted group selected from alkyl, cycloalkyl, alkoxy, cycloalkoxy, aryl, aralkyl, alkylcarbonyl, alkoxycarbonyl, arylcarbonyl, aryloxycarbonyl, aralkoxycarbonyl, heteroarylcarbonyl, aryloxy, aralkoxy, alkylcarbonyloxy, alkoxycarbonylamino, aryloxycarbonylamino, aralkoxycarbonylamino, heteroarylcarbonylamino, heteroaryl, heteroaralkyl, heterocyclyl, heteroaralkoxy, heteroaryloxy, fluorenylmethoxycarbonyl (Fmoc), fluorenylmethoxycarbonylamino (N-Fmoc), —OSO₂R⁸, —OCONR⁸R⁹, NR⁸COOR⁹, —NR⁸COR⁹, —NR⁸R⁹, —NR⁸SO₂R⁹, —NR⁸CONR⁹R¹⁰, —NR⁸CSNR⁸R⁹, —SO₂R⁸, —SOR⁸, —SR⁸, —SO₂NR⁸R⁹, —SO₂R⁸, —CONR⁸R⁹, —COOR⁹ or —COR⁹, wherein R⁸, R⁹ and R¹⁰ may be same or different and independently represent hydrogen, optionally substituted group selected from alkyl, aryl, aralkyl, aryloxy or heteroaryl; or R¹ and R² together represent a monocyclic or polycyclic aromatic or non aromatic ring or an aromatic ring fused to a non aromatic ring, which may optionally contain 1 to 3 heteroatoms selected from N, S, or O and may be unsubstituted or have up to 1 to 4 substituents which may be identical or different
R³ and R⁴ may be same or different and independently represent hydrogen, halogen, optionally substituted group selected from alkyl, cycloalkyl, alkanoyl, aryl, aroyl, aralkyl or aralkanoyl group. ‘n’ and ‘p’ independently represents 0-6.
X represents O, S, NR where R represents hydrogen or optionally substituted groups selected from alkyl, cycloalkyl, cycloalkylalkyl, aryl, aralkyl, alkanoyl, or aroyl.
Ar represents optionally substituted single or fused aromatic, heteroaromatic or heterocyclic group.
Z represents O, S, NR where R is as defined above.
R⁵, R⁶ and R⁷ may be same or different and independently represent hydrogen, hydroxy, halogen or optionally substituted group selected from alkyl, cycloalkyl, alkoxy, aryl, aralkyl, heteroaryl, heterocyclyl or heteroaralkyl groups. R⁵ and R⁶ together may form a 5 or 6 membered cyclic rings, which may contain one or two hetero atoms selected from O, S or N.
Y represents O or NR¹¹ where R¹¹ represents hydrogen, optionally substituted group selected from alkyl, aryl, aralkyl, alkanoyl, aroyl, aralkanoyl, heterocyclyl or heteroaryl.
R⁷ and R¹¹ together may also form a 5 or 6 membered cyclic ring, which may contain one or two hetero atoms selected from O, S or N.
‘----’ represents a bond or no bond.
When the fused rings formed by R¹ and R² are substituted, the substituents are selected from (C₁-C₁₀)alkyl, halogen, hydroxy, halo(C₁-C₁₀)alkyl, nitro, amino, cyano, oxo, or thioxo.
When the groups represented by R¹ and R² are substituted, the substituents are selected from halogen, hydroxy, nitro, amino, oxo, thioxo, optionally substituted groups selected from alkyl, cycloalkyl, alkoxy, aryl, aralkyl, alkylsulfonyl, alkylsulinyl, alkylsulfanyl, alkylsulfonyloxy, alkylsulfinyloxy or alkylsulfanyloxy, the substituents are selected from halogen, hydroxyl, nitro, amino, cyano or alkyl.
When the groups represented by R, R³, R⁴, R⁷ and R¹¹ are substituted, the substituents are selected from halogen, nitro, amino, hydroxy, alkyl, oxo or aralkyl
When the groups represented by R⁵, R⁶ and R⁷ are substituted, the substitutents are selected from halogen, hydroxy, nitro, alkyl, cycloalkyl, alkoxy, aryl, aralkyl, aralkoxyalkyl, heterocyclyl, heteroaryl or amino.
When the cyclic rings formed by R⁵ and R⁶ are substituted, the substituents are selected from alkyl, halogen, hydroxy, haloalkyl, nitro, amino, cyano, oxo, or thioxo.
The groups defined for R, R¹, R², R³, R⁴, R⁵, R⁶, R⁷, R⁸, R⁹, R¹⁰ and R¹¹ may be unsubstiuted, or have 1 to 4 substituents, which may be identical or different,
which comprises the following processes:
Process (a):
(i) Reacting the compound of formula (Ia) 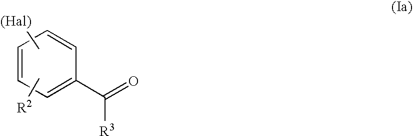
where ‘Hal’ represents a halogen atom selected bromine or iodine, R² is hydrogen and R³ is as defined above in this claim in the description of compound of formula (I), in a Witting-Horner reaction manner, by using phosphono acetate compounds selected from triethyl phosphono acetates, trimethylphosphono acetate or Ph₃P⁺—CH₂—CO₂Et in the presence of a base selected from sodium hydride, potassium tertiary butoxide, potassium hydroxide, sodium methoxide or sodium ethoxide. The solvent used in the reaction is selected from alcohol selected from methanol, ethanol, propanol, isopropanol or tetrahydrofuran, ether, dioxane, dimethoxyethane or a mixture thereof at a temperature range of 0 to 10° C. and duration of 10 to 24 h to obtain a compound of formula (Ib) 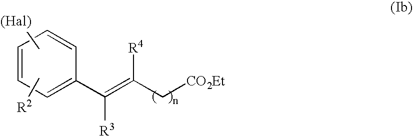
where ‘Hal’ represents a halogen atom selected bromine or iodine, R² is hydrogen and R³ and R⁴ are as defined above in this claim in the description of compound of formula (I),
(ii) conversion of the compound of formula (Ib), to a compound of formula (Ic) 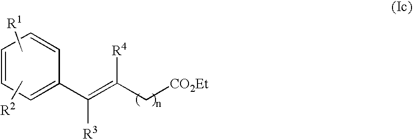
where R¹ represent aryl group, R² represents hydrogen atom and R³ and R⁴ are as defined as defined above in this claim in the description of compound of formula (I), in a Suzuki coupling reaction manner, by using aryl boronic acid with palladium catalyst like Pd(PPh₃)₄, PdCl₂, Pd(dba)₂. The solvent used is selected from terahydrofuran, dioxane, acetonitrile, dimethylether, diethylether, dimethylformamide or a mixture thereof at reflux temperature of the solvent used for a period of 15 to 28 h.
Alternatively, the compound of formula (Ic), is prepared from compound of formula (Ia′) 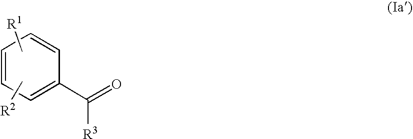
where R¹, R² and R³ are as defined above in this claim in the description of compound of formula (I), by using substituted phosphone acetate compounds selected from triethyl phosphono acetates, trimethylphosphono acetate or Ph₃P⁺—CH₂⁻—CO₂Et.
(iii) The reduction of the compound of formula (Ic) to a compound of formula (Id) 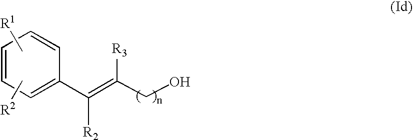
where R¹ represent aryl group, R² represents hydrogen atom and R³ and R⁴ are as defined above in this claim in the description of compound of formula (I), is carried out in the presence of a reducing agent selected from diisobutyl aluminium hydride (DIBAL-H), aluminium hydride (AlH₃) or lithium aluminium (LAH). The solvent used in the reaction is selected from toluene, tetrahydrofuran, ether, dioxane, dimethoxyethane or a mixture thereof at a temperature range of −90 to −25° C., for a duration of 0.5 h to 2 h. The temperature and duration of the reaction can be decreased in the presence of AlH₃.
(iv) coupling of a compound of formula (Id) with a compound of formula (Ie) 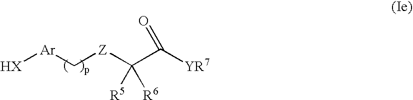
where p represents 1, Y represents O or S, R⁵ and R⁶ are as defined above in this claim in the description of compound of formula (I), R⁷ is as defined above in this claim in the description of compound of formula (I) except hydrogen, to obtain compound of formula (I), where p represents 1, Y represents O or S, R⁷ is as defined above in this claim in the description of compound of formula (I) except hydrogen atom and all other symbols are as defined above in this claim in the description of compound of formula (I), by using PPh₃, DIAD or DEAD. The solvent used in the reaction is selected from tetrahydrofuran, toluene, benzene or a mixture thereof at a temperature range of 20 to 40° C., for duration of 40 to 80 h.
(v) hydrolysis of the compound of general formula (I) where R⁷ represents hydrogen atom, Y represents O or S, p represents 1 and all other symbols are as defined above in this claim in the description of compound of formula (I), is obtained from a compound of formula (I) where R⁷ represents all groups defined above in this claim in the description of compound of formula (I) except hydrogen, Y represents O or S, p represents 1 and all other symbols are as defined above in this claim in the description of compound of formula (I), in the presence of a base selected from sodium hydroxide, potassium hydroxide, lithium hydroxide, potassium carbonate or sodium carbonate. The solvent used is selected from alcohols selected from methanol, ethanol, propanol, isopropanol or a mixture thereof, water, tetrahydrofuran, dioxane, ether or a mixture thereof at a temperature range of 30 to 80° C., for duration of 2 to 24 h.
(vi) the compound of general formula (I) where Z represents O or S, p represents 1 and R⁷ represents hydrogen or alkyl group are converted to compound of formula (I), where Y represents NR¹¹ by reacting with an amine of the formula NH R⁷R′, where R⁷ and R¹¹ are as defined above in this claim in the description of compound of formula (I), to yield a compound of formula (I) where Y represents NR¹¹ and all other symbols are as defined above in this claim in the description of compound of formula (I). Alternatively, the compound of formula (I) where YR⁷ represents OH are converted to acid halide, preferably where YR⁷ Cl, by reacting with reagents selected from oxalyl chloride or thionyl chloride, followed by treatment with an amine of the formula NHR⁷R¹¹ where R⁷ and R¹¹ are as defined above in this claim in the description of compound of formula (I). Alternatively, mixed anhydrides are obtained from compound of formula (I) where YR⁷ represents OH and all other symbols are as defined above in this claim in the description of compound of formula (I), by treating with acid halide selected from acetyl chloride, acetyl bromide, pivaloyl chloride or dichlorobenzoyl chloride. The reaction can be carried out in the presence of pyridine, triethylamine or diisopropyl ethylamine. Coupling reagent selected from DCC/DMAP DCC/HOBt, EDCI/HOBT, DIC/HOBt, ethylchloroformate, isobutylchloroformate can be used to activate the acid. The solvent used is selected from halogenated hydrocarbon like CHCl₃ or CH₂Cl₂; hydrocarbon like benzene, toluene, xylene or a mixture thereof at a temperature range of −40 to 40° C. The acid halide or mixed anhydride or activated acid obtained by coupling reagents described above thus prepared may further be treated with an amine of the formula NHR⁷R′ where R⁷ and R¹¹ are as defined above in this claim in the description of compound of formula (I), to yield a compound of formula (I) where Y represents NR¹¹ and all other symbols are as defined above in this claim in the description of compound of formula (I).
Process (b):
The reaction of compound of formula (IIa) 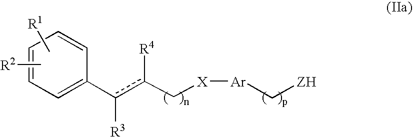
where all symbols are as defined above in this claim in the description of compound of formula (I), with a compound of formula (IIb) 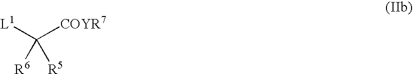
where L¹ is a leaving group selected from hydroxy, halogen atom, p-toluenesulfonate, methanesulfonate or trifluoromethanesulfonate, and all other symbols are defined above in this claim in the description of compound of formula (I), is carried out in the presence of a solvent selected from THF, DMF, DMSO, DME, toluene, benzene, xylene or a mixture thereof in the presence of a base selected from K₂CO₃, Na₂CO₃, NaNH₂, n-BuLi, NaH, KH, triethylamine, collidine, lutidine or a mixture thereof optionally in an inert atmosphere of nitrogen, helium or argon at a temperature range of 0 to 120° C., for a duration of 1 to 72 h.
Process (c):
The reaction of compound of formula (IIc) 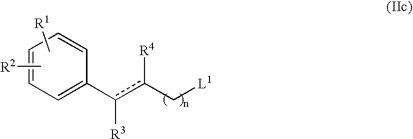
where L¹ represents a leaving group selected from hydroxy, halogen atom, p-toluenesulfonate, methanesulfonate or trifluoromethmesulfonate, and all other symbols are as defined above in this claim in the description of compound of formula (I), with compound of formula (IId) 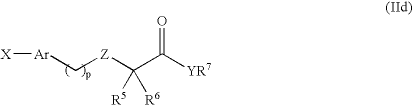
where all symbols are as defined above in this claim in the description of compound of formula (I), is carried out in the presence of a solvent selected from THF, DMF, DMSO, DME or a mixture thereof optionally in an inert atmosphere of nitrogen, argon or helium in the presence of a base selected from K₂CO₃, Na₂CO₃ or NaH, KH, triethyl amine or a mixture thereof at a temperature range of 0 to 120° C. and duration of 1 to 72 h.
or
Process (d):
The conversion of compound of formula (IIe) 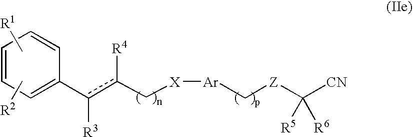
where all symbols are as defined above in this claim in the description of compound of formula (I), to a compound of formula (I), where YR⁷ represents OH and all other symbols are as defined above in this claim in the description of compound of formula (I), is carried out either in the presence of a base or an acid. Selection of base or an acid is not critical. Any base normally used for the hydrolysis of nitrile to an acid can be employed, metal hydroxide selected from NaOH or KOH in an aqueous solvent or any acid normally used for hydrolysis of nitrile to ester can be employed selected from dry HCl in an excess of alcohol like methanol, ethanol, propanol, isopropanol or a mixture thereof at a temperature range 0° C. to 150° C. and duration of 0.25 to 48 h.
Process (e):
(i) The compound of formula (IIIa) 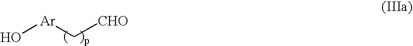
where ‘p’ and ‘Ar’ are as defined above in this claim in the description of compound of formula (I), is converted to a compound of formula (IIIb) 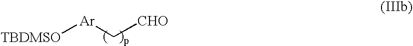
by reacting with TBDMS-Hal, (CH₃)₃Si-Hal or Ph₃C-Hal where ‘Hal’ represents halogen atom in the presence of a base used selected from triethylamine, Na₂CO₃ or K₂CO₃ and a solvent selected from dichloromethane, tetrahydrofuran, chloroform, dimethylether, diethylether, dioxane, benzene, toluene or a mixture thereof at a temperature range of 0° C. to room temperature and duration of 8 to 20 h.
(ii) The compound of formula (IIIb) is converted to a compound of formula (IIIc) 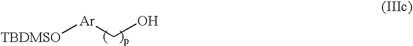
by using NaBH₄ in the presence of an alcohol selected from methanol, ethanol, propanol, isopropanol or a mixture thereof as a solvent at room temperature for a duration of 1 to 4 h.
(iii) The compound of formula (IIIc) is converted to a compound of formula (IIId) 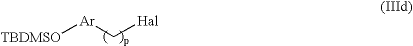
in the presence of C(Hal)₄, where ‘Hal’ represents halogen atom in the presence of PPh₃ and a solvent selected from dichloromethane, tetrahydrofuran, chloroform, dimethylether, diethylether, dioxane, benzene, toluene or a mixture thereof at room temperature for a duration of 0.5 to 2 h.
(iv) The compound of formula (IIId) is reacted with the compound of formula (IIIe) 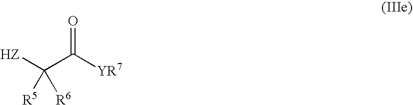
where all the symbols are as defined above in this claim in the description of compound of formula (I), to obtain a compound of formula (IIIf) 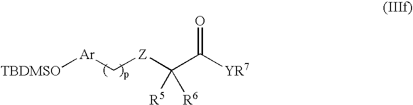
where all the symbols are as defined above in this claim in the description of compound of formula (I). The reaction is carried out in the presence of a base selected from NaH, KH, sodium amide or potassium tertiary butoxide in the presence of a selected from DMSO, THF, toluene, benzene or a mixture thereof at a temperature range of 50 to 90° C., for a period of 8 to 15 h.
(v) The deprotection of compound of formula (IIIf) to obtain a compound of formula (IIIg) 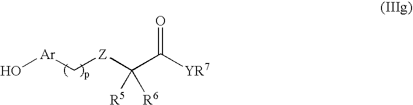
where all the symbols are as defined above in this claim in the description of compound of formula (I), is carried out by using tetrabutylammoniumfluoride (TBAF) in the presence of a solvent selected from water, THF, dioxane, dichloromethane, chloroform, methanol, ethanol or a mixture thereof at a temperature range of 20 to 40° C. and duration of 1 to 6 h.
(vi) The compound of formula (IIIg) is reacted with the compound of formula (IIIh) 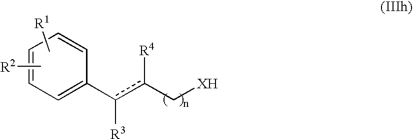
where all the symbols are as defined above in this claim in the description of compound of formula (I), to obtain a compound of formula (I), where Y represents O or S, R⁷ represents all groups as defined above in this claim in the description of compound of formula (I) except hydrogen. The reaction is carried out by using PPh₃, diisopropyl azadicarboxylate (DIAD), or diethyl azadicarboxylate (DEAD) in the presence of a solvent selected from tetrahydrofuran, toluene, benzene or a mixture thereof at a temperature range of 20 to 40° C. and duration of 40 to 80 h.
(vii) The compound of general formula (I) where R⁷ represents hydrogen atom, Y represents O or S, p represents I and all other symbols axe as defined above in this claim in the description of compound of formula (I), is obtained from compound of formula (I) where R⁷ is as defined above in this claim in the description of compound of formula (I) except hydrogen, Y represents O or S, p represents 1 and all other symbols are as defined above in this claim in the description of compound of formula (I), by hydrolysis using conventional methods. The reaction is carried out in the presence of a base selected from sodium hydroxide, potassium hydroxide, lithium hydroxide, potassium carbonate or sodium carbonate in the presence of a solvent alcohol like methanol, ethanol, propanol, isopropanol or a mixture thereof, water, tetrahydrofuran, dioxane, ether or a mixture thereof at a temperature range of 30 to 80° C. and duration of 2 to 24 h.

38. A process for the preparation of resolution of compound of formula (I), 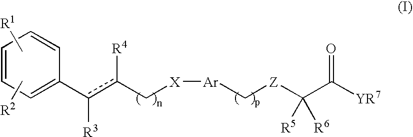
wherein R¹ and R² may be same or different and independently represent hydrogen, halogen, nitro, cyano, amino, hydroxy or optionally substituted group selected from alkyl, cycloalkyl, alkoxy, cycloalkoxy, aryl, aralkyl, alkylcarbonyl, alkoxycarbonyl, arylcarbonyl, aryloxycarbonyl, aralkoxycarbonyl, heteroarylcarbonyl, aryloxy, aralkoxy, alkylcarbonyloxy, alkoxycarbonylamino, aryloxycarbonylamino, aralkoxycarbonylamino, heteroarylcarbonylamino, heteroaryl, heteroaralkyl, heterocyclyl, heteroaralkoxy, heteroaryloxy, fluorenylmethoxycarbonyl (Fmoc), fluorenylmethoxycarbonylamino (N-Fmoc), —OSO₂R⁸, —OCONR⁸R⁹, NR⁸COOR⁹, —NR⁸COR⁹, —NR⁸R⁹, —NR⁸SO₂R⁹, —NR⁸CONR⁹R¹⁰, —NR⁸CSNR⁹, —SO₂R⁸, —SOR⁸, —SR⁸, —SO₂NR⁸R⁹, —SO₂OR⁸, —CONR⁸R⁹, —COOR⁹ or —COR⁹, wherein R⁸, R⁹ and R¹⁰ may be same or different and independently represent hydrogen, optionally substituted group selected from alkyl, aryl, aralkyl, aryloxy or heteroaryl; or R¹ and R² together represent a monocyclic or polycyclic aromatic or non aromatic ring or an aromatic ring fused to a non aromatic ring, which may optionally contain 1 to 3 heteroatoms selected from N, S, or O and may be unsubstituted or have up to 1 to 4 substituents which may be identical or different.
R³ and R⁴ may be same or different and independently represent hydrogen, halogen, optionally substituted group selected from alkyl, cycloalkyl, alkanoyl, aryl, aroyl, aralkyl or aralkanoyl group. ‘n’ and ‘p’ independently represents 0-6.
X represents O, S, NR where R represents hydrogen or optionally substituted groups selected from alkyl, cycloalkyl, cycloalkylalkyl, aryl, aralkyl, alkanoyl, or aroyl.
Ar represents optionally substituted single or fused aromatic, heteroaromatic or heterocyclic group.
Z represents O, S, NR where R is as defined above.
R⁵, R⁶ and R⁷ may be same or different and independently represent hydrogen, hydroxy, halogen or optionally substituted group selected from alkyl, cycloalkyl, alkoxy, aryl, aralkyl, heteroaryl, heterocyclyl or heteroaralkyl groups. R⁵ and R⁶ together may form a 5 or 6 membered cyclic rings, which may contain one or two hetero atoms selected from O, S or N.
Y represents O or NR¹¹ where R¹¹ represents hydrogen, optionally substituted group selected from alkyl, aryl, aralkyl, alkanoyl, aroyl, aralkanoyl, heterocyclyl or heteroaryl.
R⁷ and R¹¹ together may also form a 5 or 6 membered cyclic ring, which may contain one or two hetero atoms selected from O, S or N.
‘----’ represents a bond or no bond.
When the fused rings formed by R¹ and R² are substituted, the substituents are selected from (C₁-C₁₀)alkyl, halogen, hydroxy, halo(C₁-C₁₀)alkyl, nitro, amino, cyano, oxo, or thioxo.
When the groups represented by R¹ and R² are substituted, the substituents are selected from halogen, hydroxy, nitro, amino, oxo, thioxo, optionally substituted groups selected from alkyl, cycloalkyl, alkoxy, aryl, aralkyl, alkylsulfonyl, alkylsulinyl, alkylsulfanyl, alkylsulfonyloxy, alkylsulfinyloxy or alkylsulfanyloxy, the substituents are selected from halogen, hydroxyl, nitro, amino, cyano or alkyl.
When the groups represented by R, R³, R⁴, R⁷ and R¹¹ are substituted, the substituents are selected from halogen, nitro, amino, hydroxy, alkyl, oxo or aralkyl
When the groups represented by R⁵, R¹ and R⁷ are substituted, the substitutents are selected from halogen, hydroxy, nitro, alkyl, cycloalkyl, alkoxy, aryl, aralkyl, aralkoxyalkyl, heterocyclyl, heteroaryl or amino.
When the cyclic rings formed by R⁵ and R⁶ are substituted, the substituents are selected from alkyl, halogen, hydroxy, haloalkyl, nitro, amino, cyano, oxo, or thioxo.
The groups defined for R, R¹, R², R³, R⁴, R⁵, R⁶, R⁷, R⁸, R⁹, R¹⁰ and R¹¹ may be unsubstiuted, or have 1 to 4 substituents, which may be identical or different, to obtain substantially pure compounds of formula (Ii) and (Iii) 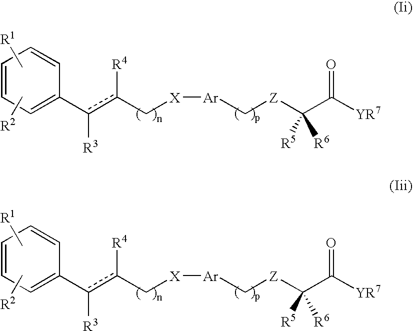
where all symbols are as defined above,
by using chiral base selected from S(+)-α-methylbenzylamine, R(−)-α-methylbenzylamine, S(+)-lysine; R(−)-lysine, S(+)—N-methyl-D-glucamine, R(−)—N-methyl-D-glucamine, R(−)-phenyl glycinol, S(+)-phenyl glycinol, S(+)-brucine, R(−)-brucine, cinchona alkaloids and their derivatives

39. The process as claimed in claim 38, wherein the chiral base is selected from S(+)-phenolgycenol, R(−)-phenolgycenol.

40. A pharmaceutical composition, which comprises a compound of formula (I) 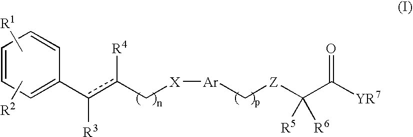
as defined in claim 1 and a pharmaceutically acceptable carrier, diluent, excipient or solvate.

41. The pharmaceutical composistion of claim 40, wherein the compound is

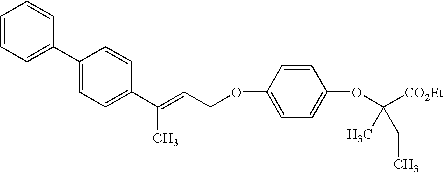

42. The pharmaceutical composistion of claim 40, wherein the compound is

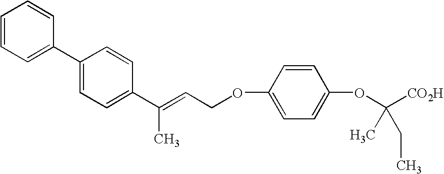

43. The pharmaceutical composistion of claim 40, wherein the compound is

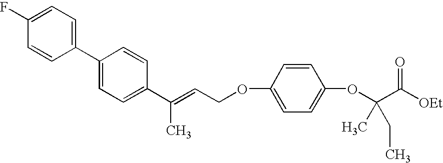

44. The pharmaceutical composistion of claim 40, wherein the compound is

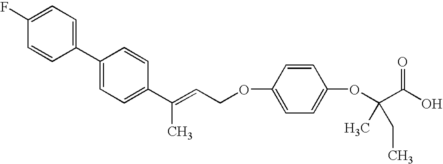

45. The pharmaceutical composistion of claim 40, wherein the compound is

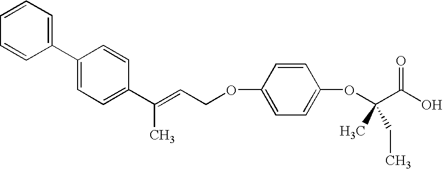

46. The pharmaceutical composistion of claim 40, wherein the compound is

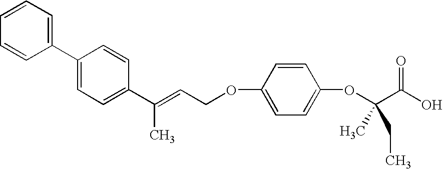

47. The pharmaceutical composistion of claim 40, wherein the compound is

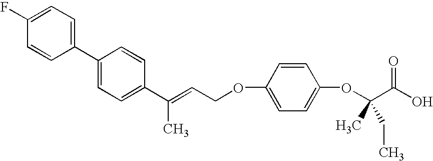

48. The pharmaceutical composistion of claim 40, wherein the compound is

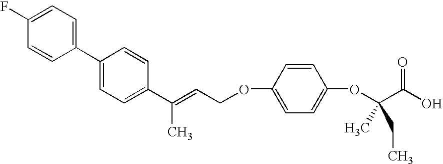

49. The pharmaceutical composistion of claim 40, wherein the compound is

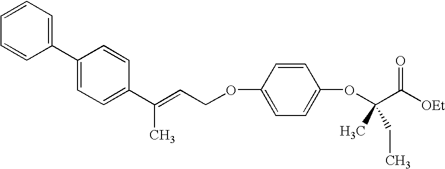

50. The pharmaceutical composistion of claim 40, wherein the compound is

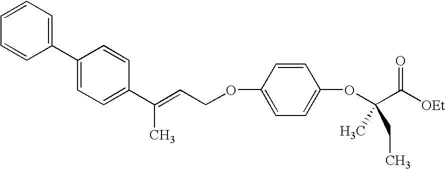

51. The pharmaceutical composistion of claim 40, wherein the compound is

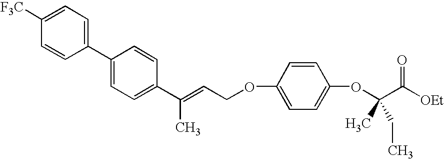

52. The pharmaceutical composistion of claim 40, wherein the compound is

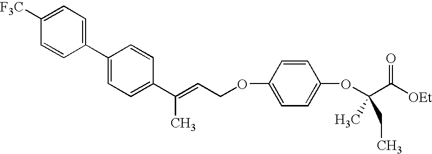

53. The pharmaceutical composition as claimed in claim 40 in the form of a tablet, capsule, powder, syrup, solution or suspension.

54. A method for treating and/or preventing dyslipidemia comprising administering a compound of formula (I) as defined in claim 1 or a pharmaceutical composition according to claim 40 to a patient in need thereof.

55. A method for treating and/or preventing diabetes caused by insulin resistance or impaired glucose tolerance comprising administering a compound of formula (I) as defined in claim 1 or a pharmaceutical composition according to claim 40 to a patient in need thereof.

56. Use of a compound of formula (I) as defined in claim 1 or a pharmaceutical composition according to claim 40 for treating and/or preventing dyslipidemia.

57. Use of a compound of formula (I) as defined in claim 1 or a pharmaceutical composition according to claim 40 for treating and/or preventing diabetes caused by insulin resistance or impaired glucose tolerance.

58. A medicine for treating and/or preventing diabetes caused dyslipidemia comprising administering a compound of formula (I) as defined in claim 1 or a pharmaceutical composition according to claim 40 to a patient in need thereof

59. A medicine for treating and/or preventing diabetes caused by insulin resistance or impaired glucose tolerance comprising administering a compound of formula (I) as defined in claim 1 or a pharmaceutical composition according to claim 40 to a patient in need thereof.
